(19) United States
(12) Patent Application Publication
(10) Pub. No.: US 2026/0049283 A1
(43) Pub. Date: Feb. 19, 2026

(54) HEAT SHOCK INDUCIBLE CELLS AND THEIR MANUFACTURE AND USE

(71) Applicant: Upside Foods, Inc., Berkeley, CA (US)

(72) Inventors: Evan William Cory, Oakland, CA (US); Rachel Anne Valenzuela, El Cerrito, CA (US)

(21) Appl. No.: 19/300,583

(22) Filed: Aug. 14, 2025

Related U.S. Application Data
(60) Provisional application No. 63/683,555, filed on Aug. 15, 2024.

(51) Int. Cl. C12N 5/077; A23L 13/00

(52) U.S. Cl. CPC C12N 5/0658; A23L 13/00; C12N 2510/00

(57) ABSTRACT

Provided are methods and systems for producing a cell based product for dietary consumption, methods and systems for selecting cell lines with heat shock inducible genes associated with a fat or muscle phenotype, draw and fill methods and systems, methods and systems for a chemostat with cell retention, chicken or bovine cell lines, and edible bovine or avian cell lines obtained from the methods described herein.

### 0. SEQUENCE LISTING

The contents of the electronic sequence listing named “38279.0019U2.xml,” created on Aug. 14, 2025 and having a size of 55,405 is hereby incorporated by reference in its entirety.

### 1. FIELD OF INVENTION

Provided are methods and systems for producing animal cell based food products, methods and systems for selecting cell lines with heat shock inducible gene expression systems, a draw and fill method, methods and systems for a chemostat with cell retention, and edible bovine and avian cell lines obtained from the methods described herein.

### 2. BACKGROUND OF INVENTION

Food products formulated from cells cultured in vitro offer potential advantages over food products obtained from animal slaughter. Production of food products formulated from cultured cells has been projected to require fewer resources, convert biomass at a higher caloric efficiency, and result in reduced environmental impacts relative to conventional in vivo methods. Together, metazoan cells, and their extracellular products, constitute a biomass that can be harvested from a culture vessel for formulation of cell-based food products, such as cultured meat.

However, several challenges persist, such as the mass production of cells for formulating into cell-based food products and the optimization of the moisture content, texture profile, and flavor of the resulting cell-based food product. Current formulations of cell-based food products often lack one or more of an ideal moisture content, texture profile, and flavor necessary to make cultivated meat formulations indistinguishable from popular, conventionally slaughtered meat that has been prepared for consumption.

In culturing cells for production of cell-based meat products, the cells are cultured to a certain cell density and then ideally induced to express phenotypes associated with muscle, fat, or some combination thereof, e.g., through genes conducive to cell specialization. However, controlling the expression of genes of interest in cells is a non-trivial task with both scientific and logistical complexity. For instance, different cell types require different physical conditions, different media components (e.g., growth factors), different activated receptors, and often some combination thereof. Furthermore, the complexities of controlled gene expression are amplified by the unique restrictions of producing cultivated meat-which do not apply to simple recombinant protein expression or bacterial cultures-including the requirement that only edible materials and food friendly processes are used, in combination with the inherent challenges, fragility, and slow growing nature when culturing avian, mammalian, fish, and crustacean cell types. Current techniques for inducible protein expression include inducible promoter systems, such as doxycycline or tetracycline induction systems. However, using antibiotics or other exogenous agents for inducing differentiation also introduces these exogenous factors into the cell culture medium, and these ingredients are typically unsafe for consumption or their safety is unknown. What is needed is a method for inducing expression of differentiation factors in the cell cultures that does not involve introduction of agents or processes that are not safe for consumption or otherwise undesirable in the production of foodstuffs and that which can be performed reliably at commercial scales of 2,000 liters or more.

Inducible heat shock promoters have been used for recombinant protein expression in lower organisms, e.g., Escherichia coli. However, heat shock promoters have not been used for modulating receptor activation, modulating receptor growth, producing growth factors, producing growth factor ligands, inducing expression of genes associated with fat or muscle phenotype, or inducing differentiation of cells for the production of cell-based food products. Accordingly, there remains a need for food-safe processes which yield cells expressing desired critical proteins, and their associated textures and flavors, common to conventional animal tissue, muscle tissue, fatty tissue, muscle cells, and fat cells.

### 3. SUMMARY OF INVENTION

The subject matter disclosed herein is based upon the inventors' discovery that heat shock promoters can be used for inducible expression of one or more nucleic acids (including genes, e.g., encoding factors which induce certain cell phenotypes), in cultured metazoan cells to permit induction by exposure to heat shock temperature of expression of one or more nucleic acids or genes, e.g., encoding proteins or nucleic acids, including differentiation factors and biomarkers, which function to promote expression of phenotypes associated with muscle, fat, etc., after a period of cell division and growth of pre-induced cells which have low to undetectable expression of such nucleic acids and phenotypes and maintain doubling times before exposure to heat shock temperatures to induce expression.

Provided are methods for cultivating a non-human animal cell culture, the method comprising: cultivating avian, bovine, or non-human mammalian cells comprising a nucleic acid encoding a factor or biomarker associated with a fat or muscle phenotype operably linked to a heat shock promoter in media in a bioreactor for a first time period at a proliferation temperature; exposing the cells to a heat shock temperature for a second time period, thereby inducing expression of the factor or biomarker; separating at least a portion of the heat shocked cells from the media; and harvesting the separated, heat shocked cells for formulation into a comestible food product.

Provided are nucleic acid vectors comprising a nucleic acid encoding a protein or nucleic acid associated with a fat or muscle phenotype, e.g., a differentiation factor and/or a biomarker, operably linked to a heat shock promoter or portion of a heat shock promoter (including a eukaryotic heat shock promoter) that promotes gene expression in cells exposed to temperatures above the cell culture temperature. Provided also are cells and cell lines comprising the nucleic acid encoding one or more proteins or nucleic acids associated with a fat or muscle phenotype, e.g., a differentiation factor or a biomarker, operably linked to the heat shock promoter or active portion thereof, and methods of producing cell based food products with cells comprising the nucleic acid vectors. In embodiments, the nucleic acid comprises nucleotide sequences encoding two, three, or four protein products or nucleic acids associated with a fat and/or muscle phenotype, all of which are operably linked to a native or integrated, heterologous or homologous heat shock promoter.

Provided herein are methods for producing a cell based product for dietary consumption, the method comprising: culturing avian, bovine, or mammalian (non-human) cells at a first temperature for a first time, which cells comprise a nucleic acid encoding a protein or nucleic acid associated with a fat or muscle phenotype operably linked to a heat shock promoter (including either transiently transfected cells or those stably incorporating such a nucleic acid), such that the cells proliferate to form a biomass but do not express the protein or nucleic acid or the phenotype to a significant degree and/or do not exhibit an increase (by any more than 33%) in doubling time; culturing the cells of the biomass at a second temperature for a second time that activates the eukaryotic heat shock promoter (a heat shock or an elevated temperature) and maintains cell viability, wherein, at the second temperature, the heat shock promoter induces expression of one or more nucleic acids encoding a protein or nucleic acid associated with a fat or muscle phenotype, e.g., a differentiation factor, biomarker, or other factor, to promote a cell phenotype, including a fat or muscle phenotype, and obtaining and processing the induced cells of the biomass for consumption. In embodiments, the doubling time of transfected cells, including after heat shock exposure does not increase by more than 5%, by more than 10%, by more than 15%, by more than 20%, by more than 25%, by more than 30%, or by more than 33%.

In embodiments, the avian, bovine, non-human mammalian, or non-human animal cells have been transfected with a nucleic acid encoding a protein or nucleic acid associated with a fat or muscle phenotype operably linked to a heat shock promoter or a heat shock regulatable portion thereof; successfully transfected cells are selected and either grown polyclonally or selected and then cloned and cultured monoclonally. Clonal selection allows for isolation of a cell line that exhibits high levels of expression under heat shock and/or does not exhibit significant leaky expression of the one or more nucleic acids operably linked to the heat shock promoter and associated with a fat or muscle phenotype when not induced but expresses such nucleic acids when exposed to the heat shock temperature, including at a level that can promote cell differentiation or promotes a cell phenotype, including fat or muscle cell phenotype.

Also provided are methods of selecting cell lines with heat shock inducible genes or nucleic acids encoding factors or biomarkers associated with a fat or muscle phenotype, including cell lines that do not exhibit significant leaky expression of the gene or nucleic acid when not exposed to heat shock temperatures, comprising: transfecting avian, bovine, non-human mammalian, or non-human animal cells with a vector construct comprising a nucleic acid encoding one or more factors (protein or nucleic acid) or biomarker associated with a fat or muscle phenotype operably linked to a heat shock promoter; clonally selecting cells that comprise the nucleic acid encoding the one or more factors or biomarkers associated with a fat or muscle phenotype operably linked to a heat shock promoter but do not exhibit leaky expression and/or changes in doubling times relative to cells without the construct (by more than 5%, 10%, 15%, 20%, 25%, 30% or 33%) and, optionally, further clonally selecting cells that express the one or more nucleic acids encoding factor or biomarker associated with a fat or muscle phenotype after the cells have been exposed to a heat shock temperature that maintains cell viability, wherein the one or more factors or biomarkers associated with a fat or muscle phenotype is expressed at a level that promotes expression of the fat or muscle phenotype in the cells. The selected cells form cell lines for use in the processes disclosed herein. In particular, such cells can be cultured under conditions that do not activate the heat shock promoter to obtain a biomass with a target cell number or cell density and then activating the heat shock promoter, for example, through heat shock temperature, which also maintains cell viability, to induce expression of the one or more factors or biomarkers associated with a fat or muscle phenotype and thereby induce fat and/or muscle phenotypes in the avian, bovine, non-human mammalian, or non-human animal cells, including inducing cells to differentiate to a myogenic or adipogenic lineage. The induced cells may be processed for production of cell-based meat products.

In embodiments, in cells of the cell line, the expression of one or more factors or biomarkers associated with a fat or muscle phenotype operably linked to the heat shock promoter when the cell is cultured at culture temperatures which are below temperatures that activate the heat shock promoter is less than 0.1%, 1%, 5%, or 10% of the level of expression of the one or more factors or biomarkers associated with a fat or muscle phenotype operably linked to the heat shock promoter in cells from the cell line that have been exposed to a temperature that activates the heat shock promoter. In embodiments, in cells of the cell line, the doubling time of the cells is maintained, i.e., is not increased by more than 1%, 5% or 10%, over the culture time when cultured at culture temperatures below temperatures which activate the heat shock promoter and is maintained or recovers after exposure to the heat shock temperature.

In embodiments, the avian, bovine, non-human mammalian, or non-human animal cells comprise a nucleic acid encoding a heat shock promoter operably linked to a nucleic acid encoding a heat shock factor and a nucleic acid encoding one or more factors or biomarkers associated with a fat or muscle phenotype, wherein expression of the heat shock factor and factors(s) associated with a fat or muscle phenotype is induced at a heat shock temperature, and the heat shock factor continuously activates the heat shock promoter to increase expression of the factor (protein or nucleic acid) associated with the fat or muscle phenotype.

Provided herein is a draw and fill method comprising: cultivating either polyclonal or clonally derived cells that comprise a nucleic acid encoding a protein or nucleic acid associated with a fat or muscle phenotype, e.g., a differentiation factor or biomarker, operably linked to a heat shock promoter in a bioreactor having a capacity of at least 2 kiloliters until reaching a PCV (packed cell volume) of at least 6% at a first temperature; draining at least 60% of the cell culture to a finishing system; culturing the cells in the finishing system at a second temperature, wherein the second temperature averages about 43° C. (or appropriate temperature that activates the heat shock promoter and maintains cell viability) to induce expression by the heat shock promoter of the one or more factors or biomarkers associated with a fat or muscle phenotype and, in embodiments, differentiation or induction of a fat or muscle cell phenotype in a substantial portion of the cells; separating the cells from the media; and harvesting for formulation into a comestible food product.

Also provided herein is a chemostat with a cell retention culturing method comprising: seeding an inoculum of cells comprising either polyclonal or a clonal population having a nucleic acid encoding one or more factors or biomarkers associated with a fat or muscle phenotype operably linked to a heat shock promoter; growing the cells for at least 2 days without cell recovery; and partially recovering a fraction of the cells at a steady rate, wherein the partial recovery includes either a finishing tank whose contents are held at about 43° C. or inline heating of 43° C. (or appropriate temperature that activates the heat shock promoter and maintains cell viability), inline aeration, and a transit time sufficient to differentiate or induce a fat or muscle phenotype in a substantial portion of the cells.

Provided herein also is an avian, bovine, non-human mammalian, or non-human animal cell line that comprises cells comprising a nucleic acid encoding one or more factors or biomarkers associated with a fat or muscle phenotype operably linked to a heat shock promoter, wherein the cell line is characterized by suspension adaptation, a population doubling level exceeding 100, a doubling time less than 24 hours, a culture density tolerance exceeding 1×10⁷ cells/mL, adaptation to serum free media, edibility and, heat shock inducible gene expression.

Also provided herein is a method of selectively inducing expression of one or more factors or biomarkers associated with a fat or muscle phenotype in bovine or avian cells without small molecules within a stainless steel vessel, wherein the resulting cells are edible.

Also provided herein is an edible bovine or avian cell line comprising an inducible gene expression when cultured at temperature of 43° C.

### 3.1. Illustrative Embodiments

Embodiment 1. A non-human animal cell cultivation method, comprising:
a. cultivating avian, bovine, non-human mammalian, or non-human animal cells in a bioreactor for a first time period at a proliferation temperature;
b. exposing the cells to a heat shock temperature for a second time period, thereby inducing expression of the factor or biomarker;
c. separating at least a portion of the heat shocked cells from the media; and
d. harvesting the separated, heat shocked cells for formulation into a comestible food product.

Embodiment 2. A method for producing a cell-based product for dietary consumption, comprising:
a. transfecting cells, comprising avian, bovine, non-human mammalian, or non-human animal cells, with a nucleic acid encoding a protein or nucleic acid associated with a fat or muscle phenotype operably linked to a heat shock promoter;
b. selecting successfully transfected cells;
c. culturing the transfected cells at a heat shock temperature that activates the heat shock promoter and maintains cell viability for a time period, wherein the activated heat shock promoter induces expression of the fat or muscle phenotype; and
d. obtaining and processing the cells expressing the fat or muscle phenotype for consumption.

Embodiment 3. A method of selecting cell lines with an inducible heat shock system, comprising:
a. transfecting cells, comprising avian, bovine, non-human mammalian, or non-human animal cells, with a vector construct comprising a nucleic acid encoding a fluorescent protein and a heat shock promoter operably linked to a nucleic acid encoding a factor of interest, wherein the nucleic acid encoding the fluorescent protein is substantially unintegrated; and
b. selecting cells expressing the fluorescent protein after a first period; and
c. discarding cells expressing the fluorescent protein after a second period, wherein a substantial majority of remaining cells express factor operably linked to the heat shock promoter and not the fluorescent protein.

Embodiment 4. The method of producing a cell line with heat shock inducible gene, comprising:
a. transfecting cells, comprising avian, bovine, non-human mammalian, or non-human animal cells with a vector construct comprising a nucleic acid encoding a factor operably linked to a heat shock promoter;
b. discarding cells that express the nucleic acid operably linked to the heat shock promoter at proliferation temperatures, wherein remaining cells are cultured further; and
c. culturing the remaining cells at a heat shock temperature for a time period that activates the heat shock promoter,
d. wherein less than 10% of the remaining cells express the nucleic acid operably linked to the heat shock promoter at or below 37° C.

Embodiment 5. A draw and fill cell culture method, comprising:
a. cultivating clonally derived non-human mammalian or non-human animal cells for a first time at a first temperature in a bioreactor having a capacity of at least 20 kiloliters until reaching a PCV of at least 6%;
b. draining at least 60% of the cell culture to a differentiation system;
c. culturing the cells in the differentiation system for a second time at a second temperature to induce differentiation in a substantial portion of the cells;
d. separating the cells from the media; and
e. harvesting the cells for formulation into a comestible food product.

Embodiment 6. A chemostat with cell retention culturing method comprising:
a. seeding an inoculum of non-human mammalian or non-human animal cells comprising a clonal population having an integrated differentiation factor operably linked to a heat shock promoter;
b. growing the cells for at least 2 days without cell recovery; and
c. partially recovering a fraction of the cells at a steady rate, wherein the partially recovery includes inline heating to 43° C., inline aeration, and a transit time sufficient to differentiate a substantial portion of the cells.

Embodiment 7. The method of any one of embodiments 1-6, wherein the cells comprise a nucleic acid encoding a protein or nucleic acid associated with a fat or muscle phenotype and said nucleic acid is operably linked to a heat shock promoter or wherein the cells have been transfected with a vector construct comprising a nucleic acid encoding a protein or nucleic acid associated with a fat or muscle phenotype operably linked to a heat shock promoter.

Embodiment 8. The method of any one of embodiments 1-7, wherein the vector construct does not comprise an antibiotic resistance gene.

Embodiment 9. The method of any one of embodiments 1-8, wherein the cells have a basal doubling time before exposure to the heat shock temperature, wherein the cells return to the basal doubling time within 6 days of exposure to the heat shock temperature.

Embodiment 10. The method of any one of embodiments 1-9, wherein the heat shock promoter is homologous or heterologous to the species of the cells.

Embodiment 11. The method of any one of embodiments 1-1,0 wherein the heat shock promoter is a portion of the heat shock promoter which has heat-inducible promoter activity.

Embodiment 12. The method of any one of embodiments 1-11, wherein the heat shock promoter is modified to exhibit reduced activation by heavy metals or ultraviolet light as compared to a heterologous or homologous heat shock promoter.

Embodiment 13. The method of any one of embodiments 1-12, wherein the factor or biomarker associated with a fat phenotype is PPARG and/or CEBPA.

Embodiment 14. The method of any one of embodiments 1-12, wherein the factor or biomarker associated with a muscle phenotype is PAX7, MYOD, MYH2 and/or MEF2B (MERFs).

Embodiment 15. The method of any one of embodiments 1-14, wherein the heat shock temperature or the second temperature is at or between 37° C. and 43° C.

Embodiment 16. The method of any one of embodiments 1-15, wherein the cells are cultured at the heat shock temperature or the second temperature for about 3 hours, for about 4 hours, for about 5 hours, for about 6 hours, or for about 3 hours to about 6 hours.

Embodiment 17. The method of any one of embodiments 1-16, wherein a substantial portion of the cellular population exhibits expression of the nucleic acid operably linked to the heat shock promoter at 43° C.

Embodiment 18. The method of any one of embodiments 1-17, wherein the substantial portion is at least 51% of the cellular population.

Embodiment 19. The method of any one of embodiments 1-18, wherein the cells are transferred to a differentiation system for the second time period, wherein the differentiation system is a tank the cells are cultured in or a pipe the cells flow through.

Embodiment 20. The method of any one of embodiments 1-19, wherein the bioreactor has a capacity between about 1,000 and 100,000 liters.

Embodiment 21. The method of any one of embodiments 1-20, wherein the avian cells, non-human mammalian, or non-human animal cells are chicken.

Embodiment 22. The method of any one of embodiments 1-21 wherein the avian, bovine, non-human mammalian, or non-human animal cells are fibroblasts.

Embodiment 23. The method of any one of embodiments 1-22, wherein the nucleic acid encoding a factor or biomarker associated with a muscle or fat phenotype operably linked to a heat shock promoter is also linked to a nucleic acid encoding a heat shock factor.

Embodiment 24. The method of any one of embodiments 1-23, wherein the avian, bovine, non-human mammalian, or non-human animal cells are transfected with a vector comprising or comprise a nucleic acid encoding PPARG, CEBPA, PAX7, MYOD, MYH2 and/or MEF2B (MERFs) operably linked to a heat shock promoter.

Embodiment 25. The method of any one of embodiments 1-24, wherein the avian, bovine, non-human mammalian, or non-human animal cells are transfected with a vector comprising or comprise a nucleic acid encoding MYOD and MEF2B (MERFs) operably linked to a heat shock promoter.

Embodiment 26. The method of any one of embodiments 1-25, wherein avian, bovine, non-human mammalian, or non-human animal cells are transfected with a vector comprising or comprise a nucleic acid encoding PPARG and/or CEBPA operably linked to a heat shock promoter.

Embodiment 27. The method of any one of embodiments 1-26, wherein the avian, bovine, non-human mammalian, or non-human animal cells are transfected with a vector comprising or comprise a nucleic acid encoding MYH2 operably linked to a heat shock promoter.

Embodiment 28. The method of embodiment 27, wherein MYH2 expression is induced with or without a heat shock.

Embodiment 29. The method of any one of embodiments 1-28, wherein the vector construct used to transfect the avian, bovine, non-human mammalian, or non-human animal cells comprise a nucleic acid comprising: (a) a HSP1A1 promoter, and (b) a nucleic acid encoding PPARG, CEBPA, PAX7, MYOD, MYH2 or MEF2B operably linked to the HSP1A1 promoter.

Embodiment 30. The method of any one of embodiments 1-29, wherein the cultured cells are in suspension, adherent, in suspension aggregates, or on suspension microcarriers.

Embodiment 31. The method of any one of embodiments 1-30, wherein the cells are cultivated for a finishing time period, at a proliferation temperature, for between 1 and 14 days after exposure to the heat shock temperature, wherein the cells progressively express phenotypes associated with fat or muscle cells during the finishing time period.

Embodiment 32. The method of any one of embodiments 1-31, wherein the avian, bovine, non-human mammalian, or non-human animal cells are collected and formed into an edible food product.

Embodiment 33. The method of any one of embodiments 1-32, wherein the cells do not exhibit an increase of doubling time of more than 5%, 10%, 15%, 20%, 25%, 30% or 33%.

Embodiment 34. The method of any one of embodiments 1-33, wherein the heat shock promoter has the nucleotide sequence of SEQ ID: 29.

Embodiment 35. A fibroblast cell line, comprising:
a. chicken or bovine fibroblast cells;
b. suspension adaptation;
c. a population doubling level exceeding 100;
d. a doubling time less than 24 hours;
e. a culture density tolerance exceeding 1×10⁷ cells/ml;
f. adaptation to serum free media; and
g. edibility; and
h. heat shock inducible gene expression.

Embodiment 36. An edible avian, bovine, non-human mammalian, or non-human animal cell line comprising: inducible, gene expression when cultured at temperatures of 43° C.

Embodiment 37. The cell line of embodiment 36, wherein the cells comprise a nucleic acid encoding a protein or nucleic acid associated with a fat or muscle phenotype and said nucleic acid is operably linked to a heat shock promoter or wherein the cells have been transfected with a vector construct comprising a nucleic acid encoding a protein or nucleic acid associated with a fat or muscle phenotype operably linked to a heat shock promoter.

Embodiment 38. The cell line of embodiment 36 or 37, wherein the vector construct does not comprise an antibiotic resistance gene.

Embodiment 39. The cell line of any one of embodiments 36-38, wherein the heat shock promoter is homologous or heterologous to the cells.

Embodiment 40. The cell line of any one of embodiments 36-49, wherein the heat shock promoter is a portion of the heat shock promoter which has heat-inducible promoter activity.

Embodiment 41. The cell line of any one of embodiments 36-40, wherein the heat shock promoter is modified to exhibit reduced activation by heavy metals or ultraviolet light as compared to a homologous heat shock promoter.

Embodiment 42. The cell line of any one of embodiments 36-41, wherein a substantial portion of the cellular population exhibits expression of the nucleic acid operably linked to the heat shock promoter at 43° C.

Embodiment 43. The cell line of any one of embodiments 36-42, wherein the substantial portion is at least 51% of the cellular population.

Embodiment 44. The cell line of any one of embodiments 36-43, wherein cell line returns to a doubling time of less than 24 hours 5 days or less after exposure to a heat shock temperature for a time length between about 1 and 6 hours.

Embodiment 45. The cell line of any one of embodiments 36-44, wherein the inducible gene expression comprises an integrated heat shock promoter operably linked to nucleic acid encoding one or more factors or biomarkers associated with a muscle or fat phenotype.

Embodiment 46. The cell line of any one of embodiments 36-45, wherein the inducible gene expression comprises one or more nucleic acids encoding factors or biomarkers associated with a fat or muscle phenotype operably linked to a heterologous or homologous heat shock promoter.

Embodiment 47. The cell line of any one of embodiments 36-46, wherein the nucleic acid encoding a factor or biomarker associated with a muscle or fat phenotype operably linked to a heat shock promoter is also linked to a nucleic acid encoding a heat shock factor.

Embodiment 48. The cell line of any one of embodiments 36-47, wherein the cells are transfected with a vector comprising or comprise a nucleic acid encoding PPARG, CEBPA, PAX7, MYOD, MYH2 and/or MEF2B (MERFs) operably linked to a heat shock promoter.

Embodiment 49. The cell line of any one of embodiments 36-48, wherein the cells are transfected with a vector comprising or comprise a nucleic acid encoding MYOD and MEF2B (MERFs) operably linked to a heat shock promoter.

Embodiment 50. The cell line of any one of embodiments 36-49, wherein cells are transfected with a vector comprising or comprise a nucleic acid encoding PPARG and/or CEBPA operably linked to a heat shock promoter.

Embodiment 51. The cell line of any one of embodiments 36-50, wherein the cells are transfected with a vector comprising or comprise a nucleic acid encoding MYH2 operably linked to a heat shock promoter.

Embodiment 52. The cell line of embodiment 51, wherein MYH2 expression is induced with or without a heat shock.

Embodiment 53. The cell line of any one of embodiments 36-52, wherein the vector construct used to transfected the avian, bovine, non-human mammalian, or non-human animal cells comprise a nucleic acid comprising: (a) a HSP1A1 promoter, and (b) a nucleic acid encoding PPARG, CEBPA, PAX7, MYOD, MYH2 and/or MEF2B operably linked to the HSP1A1 promoter.

Embodiment 54. The cell line of any one of embodiments 36-53, wherein the cultured cells are in suspension, adherent, in suspension aggregates, or on suspension microcarriers.

Embodiment 55. The cell line of any one of embodiments 36-54, wherein the inducible gene expression comprises an integrated heat shock promoter factor.

Embodiment 56. The cell line of any one of embodiments 36-55, wherein the heat shock promoter or the integrated heat shock factor is operably linked to a heterologous or homologous heat shock promoter or an integrated heat shock promoter, wherein heat activation induces a positive feedback loop of expression.

Embodiment 57. The cell line of any one of embodiments 36-56, wherein the cells do not exhibit an increase of doubling time of more than 5%, 10%, 15%, 20%, 25%, 30% or 33%.

Embodiment 58. The cell line of any one of embodiments 36-57, wherein the heat shock promoter has the nucleotide sequence of SEQ ID: 29.

Embodiment 59. A non-human animal cell cultivation method, comprising:
a. cultivating avian, bovine, or non-human mammalian cells comprising a nucleic acid encoding a factor or biomarker associated with a fat or muscle phenotype operably linked to a heat shock promoter in media in a bioreactor for a first time period at a proliferation temperature;
b. exposing the cells to a heat shock temperature for a second time period, thereby inducing expression of the factor or biomarker;
c. separating at least a portion of the heat shocked cells from the media; and
d. harvesting the separated, heat shocked cells for formulation into a comestible food product.

Embodiment 60. The method of embodiment 59, wherein the cells are cultivated at a proliferation temperature between 35° C. and 40° C. to a target cell density.

Embodiment 61. The method of embodiment 59 or 60, the cells are cultivated to a target cell density of about 0.1 mil/mL cells to about 70 mil/mL.

Embodiment 62. The method of any one of embodiments 59-61, wherein the cells are exposed to a heat shock temperature at or between 37° C. and 43° C. and the second time period is about 3 hours, and wherein the heat shock temperature is at least 3° C. higher than the proliferation temperature.

Embodiment 63. The method of any one of embodiments 59-62, wherein a substantial portion of the cellular population exhibits expression of the nucleic acid operably linked to the heat shock promoter at 43° C.

Embodiment 64. The method of any one of embodiments 59-63, wherein less than 10% of the cells express the nucleic acid operably linked to the heat shock promoter at or below 37° C.

Embodiment 65. The method of any one of embodiments 59-64, wherein the cells have a basal doubling time of 24 hours, and wherein the cells return to the basal doubling time within 6 days of exposure to the heat shock temperature.

Embodiment 66. The method of any one of embodiments 59-65, wherein the heat shock promoter is a HSP1A1 promoter.

Embodiment 67. The method of any one of embodiments 59-66, wherein the heat shock promoter is homologous to the species of the cells and comprises one or more portions of the heat shock promoter having heat-inducible promoter activity.

Embodiment 68. The method of any one of embodiments 59-67, wherein the heat shock promoter is modified to exhibit reduced activation by heavy metals or ultraviolet light as compared to a native heat shock promoter.

Embodiment 69. The method of any one of embodiments 59-68, wherein the factor or biomarker associated with a fat phenotype is PPARG and/or CEBPA or the factor or biomarker associated with a muscle phenotype is PAX7, MYOD, MYH2 and/or MEF2B (MERFs).

Embodiment 70. The method of any one of embodiments 59-69, wherein step b is carried out in a differentiation system for the second time period, wherein the differentiation system comprises culturing the cells in a tank or in a pipe through which the cells flow.

Embodiment 71. The method of any one of embodiments 59-70, wherein after step b, the cells are cultivated for a finishing time period, at a proliferation time between 1 and 14 days.

Embodiment 72. The method of any one of embodiments 59-71, wherein prior to step d, the cells exhibit a 0.5 to 30 fold increase in expression of factors or biomarkers associated with a fat or muscle phenotype compared to non-heat shocked cells.

Embodiment 73. The method of any one of embodiments 59-72, wherein prior to step d, the cells exhibit phenotypic and genotypic characteristics of adipocytes and/or myotubes.

Embodiment 74. A chicken or bovine fibroblast cell line, characterized by:
a. suspension adaptation;
b. a population doubling level exceeding 100;
c. a doubling time less than 24 hours;
d. a culture density tolerance exceeding 1×10⁷ cells/ml;
e. adaptation to serum free media;
f. edibility; and
g. heat shock inducible gene expression.

Embodiment 75. The cell line of embodiment 74, wherein the cell line returns to a doubling time of less than 24 hours 6 days or less after exposure to a heat shock temperature for a time length between about 1 and 6 hours.

Embodiment 76. The cell line of embodiment 74 or 75, wherein heat shock inducible gene expression is induced at a temperature at or between 37° C. and 43° C.

Embodiment 77. The cell line of any one of embodiments 74-76, wherein the inducible gene expression comprises one or more genes associated with a fat or muscle phenotype operably linked to a heat shock promoter.

Embodiment 78. The cell line of claim 19, wherein the factor or biomarker associated with a fat phenotype is PPARG and/or CEBPA or the factor or biomarker associated with a muscle phenotype is PAX7, MYOD, MYH2 and/or MEF2B (MERFs).

### 4. BRIEF DESCRIPTION OF THE DRAWINGS

These and other features, aspects, and advantages of the present invention will become better understood with regard to the following description, and accompanying drawings, where:

FIGS. 1A-1B show exemplary embodiments of the bioreactor system for the draw and fill method.

FIG. 2 shows a suspension bioreactor system for continuous cultivation and recovery of comestible non-human animal cells in accordance with one or more implementations of the present disclosure.

FIG. 3 shows an exemplary plasmid vector containing a differentiation factor operably linked to a heat shock promoter.

FIG. 4 shows fluorescence imaging of transfected Bovine 4CT HSP70 Inducible Reporter Line. Calcein staining and mRuby expression denote cell viability and heat shock induced expression, respectively. Cells were cultured at 43° C. and observed over 24 hours.

FIG. 5 shows median fluorescence intensity (MFI) in Bovine 4CT HSP70 Inducible Reporter Line incubated at 43° C. for up to 24 hours.

FIG. 6 shows fluorescence imaging of transfected Bovine 4CT HSP70 Inducible Reporter Line. Calcein staining and mRuby expression denote cell viability and heat shock induced expression, respectively. Cells were cultured at 37° C., 39° C., 41° C., and 43° C. and observed at 6 hours.

FIG. 7 shows median fluorescence intensity (MFI) in Bovine 4CT HSP70 Inducible Reporter Line incubated at 37° C., 39° C., 41° C., and 43° C. for 6 hours.

FIGS. 8A-8B show median fluorescence intensity (MFI) of (A) unsorted cells and cells sorted to reduce leaky expression (Bovine 4CT HSP70 Inducible Reporter) before heat shock and (B) after heat shock at 43° C. for 6 hours.

FIGS. 9A-9B show (A) histogram representing mRuby fluorescence intensity of non-heat shocked and heat shocked Bovine 4CT cells and (B) bar graph of MFI in non-heat shocked and heat shocked Bovine 4CT cells.

FIG. 10 shows doubling time recovery after heat shock.

FIGS. 11A-11B show (A) fluorescence imaging of transfected chicken 1312 btHSP70 Inducible Reporter Line (Calcein staining and mRuby expression denote cell viability and transfection efficiency, respectively) and (B) median fluorescence intensity (MFI) in chicken 1312 btHSP70 Inducible Reporter Line incubated at 39° C. (no heat shock) and 43° C. for 6 hours (heat shock).

FIGS. 12A-12C show staining for PPARG (A) immediately after heat shock, (B) 3 hours after heat shock, and (C) 6 hours after heat shock for a variety of heat shock exposure times for cells having HSP induced expression of PPARG.

FIGS. 13A-13C show qPCR data for PPARG (A) immediately after heat shock, (B) 3 hours after heat shock, and (C) 6 hours after heat shock for a variety of heat shock exposure times for cells having HSP induced expression of PPARG.

FIG. 14 shows qPCR data for MYOD expression immediately after heat shock and 24 hours after heat shock across three heat shock conditions of 3 hours, 6 hours, and two separate 3 hour shocks.

FIG. 15 shows the percentage of cells expressing MYOD after no heat shock (control), 3 hours of heat shock, 6 hours of heat shock, 3 rounds of heat shock for 3 hours, and 2 rounds of heat shock for 6 hours. Multiple rounds of heat shock were separated by an incubation period of 24 hours at 37° C.

FIG. 16 shows qPCR data for MYH2-1 or MYH2-2 expression in cells subjected to no heat shock (37° C. for 24 hours), heat shock at 43° C. for 6 hours, followed by incubation at 37° C. for 24 hours, 41° C. for 2 days, and 39° C. for 2 days.

FIGS. 17A-B shows qPCR data for MYH2-1 or MYH2-2 expression in PBV5 bovine cells subjected to no heat shock (37° C. for 24 hours), heat shock at 43° C. for 6 hours, followed by incubation at 37° C. for 24 hours, 41° C. for 2 days, and 39° C. for 2 days and shows fluorescence imaging of HSP-btMYH2-2. DAPI and Cy5 expression denote cell viability and heat shock induced expression, respectively. Cells were imaged before heat shock and 6 hours and 48 hours after heat shock at 43° C.

### 5. DETAILED DESCRIPTION OF THE INVENTION

Before describing particular embodiments in detail, it is to be understood that the disclosure is not limited to the particular embodiments described herein, which can vary. It is also to be understood that the terminology used herein is for the purpose of describing particular illustrative embodiments only and is not intended to be limiting unless otherwise defined. The terms used in this specification generally have their ordinary meaning in the art, within the context of this disclosure and in the specific context where each term is used. Certain terms are discussed below or elsewhere in the specification, to provide additional guidance to the practitioner in describing the compositions and methods of the disclosure and how to make and use them. The scope and meaning of any use of a term will be apparent from the specific context in which the term is used. As such, the definitions set forth herein are intended to provide illustrative guidance in ascertaining particular embodiments of the disclosure, without limitation to particular compositions or biological systems.

Standard techniques may be used for recombinant DNA, tissue culture, and transfection (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery.

Unless defined otherwise, all technical and scientific terms used herein have the same meaning as is commonly understood by one of ordinary skill in the art to which this disclosure belongs. In the event that there is a plurality of definitions for terms cited herein, those in this section prevail unless otherwise stated.

Where a term is provided in the singular, the inventors also contemplate aspects of the invention described by the plural of that term. As used in this specification and in the appended claims, the singular forms “a”, “an” and “the” include plural references unless the context clearly dictates otherwise, e.g., “a compound” includes a plurality of compounds. Thus, for example, a reference to “a method” includes one or more methods, and/or steps of the type described herein and/or which will become apparent to those persons skilled in the art upon reading this disclosure.

The present inventors have found that avian, bovine, non-human mammalian, or non-human animal cells, including fibroblasts, which comprise a nucleic acid encoding one or more factors or biomarkers (including proteins, peptides, and nucleic acids, such as siRNA, microRNA, and oligonucleotides) associated with a fat or muscle phenotype, which nucleic acid is operably linked to a heat shock promoter, cultured at a non-activating temperature to proliferate the cells to a desired cell number or cell density and are then induced to express the factors or biomarkers associated with a fat or muscle cell phenotype by exposing the cells to a heat shock temperature that activates the heat shock promoter, but maintains cell viability. The method advantageously uses cell lines with insubstantial leaky expression from the HSP, which does not negatively impact doubling times of the cells, at proliferation temperatures, to an amount greater than 5%, 10%, 15%, 20%, 25%, 30%, or 33% relative to the doubling time of a control cell line lacking the HSP inducible expression. In another embodiment, the expression from the HSP at proliferation temperatures does not negatively impact doubling times of the cell line to an amount greater than 1%, 2%, 3%, 4%, 5%, 6%, 7%, 8%, 9%, 10%, 15%, 20%, 25%, 30%, or 33% relative to a control cell line lacking the HSP inducible expression and the level of expression of the factor encoded by the nucleic acid operably linked to the HSP at proliferation temperatures is no more than 1%, 5% or 10% of the expression at heat shock temperatures. At heat shock temperatures, the cell lines robustly express integrated and nonintegrated (episomal) nucleic acids(s) operably linked to the HSP, to drive expression of phenotypes associated with fat and muscle cells, e.g., promoting cell differentiation. HSP driven expression is induced after a target cell density or number has been achieved. The method also permits cell lines to be transiently transfected with nucleic acids(s) operably linked to the HSP to drive temporary expression of factors, biomarkers, and/or phenotypes associated with fat and muscle cells. In addition, the method permits induction of the expression of one or more factors and/or biomarkers associated with fat and/or muscle phenotypes by a change in temperature and does not rely upon addition of antibiotics or other exogenous agents, which may be detrimental for the production of food products.

### 5.1. Methods for Producing Cell-Based Product

Provided herein are methods for producing a cultured cell-based product for dietary consumption, the method comprising: culturing avian, bovine, non-human mammalian, or non-human animal cells, which comprise a nucleic acid encoding a one or more factors or biomarkers (protein or nucleic acid) associated with a fat or muscle phenotype operably linked to a heat shock promoter (or a fragment thereof that is activated by heat and promotes gene expression), either stably integrated into the cell genome, at a random or targeted locus, or present episomally, at a temperature that does not activate the heat shock promoter such that the cells proliferate to form a biomass at a desired cell density or cell number (“proliferation temperature”); culturing the cells of the biomass at a second temperature that activates the eukaryotic heat shock promoter and maintains cell viability for a time (“heat shock temperature”), wherein the heat shock promoter induces expression of the nucleic acid(s) encoding the factor(s)s), e.g., to promote differentiation of the cells, including to muscle and/or fat tissue type, or promote muscle or fat cell phenotypes; and obtaining and processing the induced cells of the biomass for consumption. In embodiments, the cells are from an avian, bovine, non-human mammalian, or non-human animal cell line obtained by transfecting the cells with a vector comprising a nucleic acid encoding one or more factors or biomarkers associated with a fat or muscle phenotype operably linked to a heat shock promoter; selecting successfully transfected cells and identifying clonal cell lines which do not substantially express the nucleic acids encoding the factor(s) at non-activating temperatures or proliferation temperatures, i.e., do not exhibit leaky expression, but do express the nucleic acid encoding the factor or biomarker associated with muscle or fat cell phenotype after exposure to the heat shock activating temperature, wherein the factor is expressed at a level that promotes expression of a favorable phenotype, e.g. cell differentiation. The cells used in the methods herein are non-human mammalian and non-human animal cells, including but not limited to cells from fish, shellfish, cattle, pigs, sheep, goats, deer, rabbits, and poultry.

Provided for use in the methods and for generating the cell lines described herein are nucleic acids that encode one or more of the adipogenic or myogenic regulatory factors or biomarkers described herein (e.g., PPARG, CEBPA, CEBPG, SREBP1, SREBP2, FABP4, ZNF423, PCK1, RUNX1, EBF2, PLIN1, OSR1, PRRX1, LHX9, TWIST2, INSIG2, PAX7, MYOD, MEF2B, and fragments or variants thereof) operably linked to a heat shock promoter. In other embodiments, the nucleic acids encode one or more adipogenic or myogenic biomarker including but not limited to LEP, LPL, SCD1, MYH2, TNNT, MSTN, CK-M, and fragments or variants thereof operably linked to a heat shock promoter. In embodiments, the factor is from the same species as the cells or cell line. In embodiments, the expression of the factor is increased in the heat induced cells. In other embodiments, down regulation of endogenous regulatory factors is accomplished by induced expression of a factor (for example, an RNAi) that downregulates expression or activity of that factor. See Table 1 for amino acid sequences of the factors from various species useful herein. The constructs may contain nucleotide sequences encoding more than one, including, two, three, or four factors associated a fat or muscle phenotype operably linked to the heat shock promoter and which are expressed concurrently with either IRES sequences to direct translation of the individual differentiation factors or 2A cleavable linkers between the amino acid sequences of the factors to process the protein chain into the individual factors.

The cells used in the method and to produce the cell lines as described herein include avian, bovine, non-human mammalian, and non-human animal cells. In embodiments, the avian cells are chicken cells. In embodiments, the cells are fibroblasts.

As used herein, “HSP1A1 promoter” and “HSP70 promoter” are used interchangeably. In some embodiments, the heat shock promoter is a HSP70 (HSP1A1) promoter. The promoter sequence is, in embodiments, derived from the same species as the cells, i.e., is cis to the cells. In further embodiments, the heat shock promoter is derived from a different species than the cells. In some embodiments, the heat shock promoter is of avian or bovine origin and, in embodiments, the cells are avian or bovine cells, respectively.

In some embodiments, the heat shock promoter is a portion or fragment of the heat shock promoter having heat-inducible promoter activity. In some embodiments, the heat shock promoter is not activated by heavy metals or ultraviolet light (UV).

In some embodiments, the one or more factors or biomarkers associated with a muscle or fat phenotype is a differentiation factor that is adipogenic or myogenic. In other embodiments, the one or more factors or biomarkers associated with a muscle or fat phenotype is a biomarker associated with a muscle or fat phenotype.

In some embodiments, the one or more factors or biomarkers associated with a fat phenotype is PPARG or CEBPA. In some embodiments, the one or more factors or biomarkers associated with a fat phenotype are PPARG and CEBPA. In even further embodiments, the one or more biomarkers associated with a fat phenotype is LEP, LPL, and/or SCD1.

In some embodiments, the one or more factors or biomarkers associated with a muscle phenotype are PAX7, MYOD, and/or MEF2B (MERFs). In some embodiments, the one or more factors or biomarkers associated with a muscle phenotype are all of PAX7, MYOD, MYH2, and MEF2B. In other embodiments, the one or more biomarkers associated with a muscle phenotype is MYH2, TNNT, MSTN, and/or CK-M.

In embodiments, the proliferation temperature is between or at 35° C. and 40° C. In further embodiments, the proliferation temperature is between 35° C. and 40° C., between 35° C. and 39° C., between 35° C. and 38° C., between 35° C. and 37° C., or between 35° C. and 36° C. In even further embodiments, the proliferation temperature is 35° C., 36° C., 37° C., 38° C., 39° C., or 40° C. The proliferation temperature is optimized for culture of the particular cell line (e.g., to achieve a target doubling time and/or target cell density) without inducing heat shock expression. In embodiments, the heat shock temperature is between or at 37° C. and 45° C. In further embodiments, the heat shock temperature is between 37° C. and 45° C., between 37° C. and 44° C., between 37° C. and 43° C., between 37° C. and 42° C., between 37° C. and 41° C., between 37° C. and 40° C., between 37° C. and 39° C., or between 37° C. and 38° C. In even further embodiments, the heat shock temperature is 37° C., 38° C., 39° C., 40° C., 41° C., 42° C., 43° C., 44° C., and 45° C. In embodiments, the proliferation temperature range and heat shock temperature range overlap, and the factor or biomarker associated with a fat or muscle phenotype is expressed at a heat shock temperature 3° C. to 7° C. higher than the proliferation temperature. In embodiments, the heat shock temperature is at least 3° C. higher than the proliferation temperature. In some embodiments, the cells are cultured at a heat shock temperature for about 1 hour. In embodiments, the cells are cultured at a heat shock temperature for about 1 hour, for about 1.5 hours, for about 2 hours, for about 2.5 hours, for about 3 hours, for about 3.5 hours, for about 4 hours, for about 4.5 hours, for about 5 hours, for about 5.5 hours, for about 6 hours, for about 6.5 hours, for about 7 hours, for about 7.5 hours, for about 8 hours, for about 8.5 hours, for about 9 hours, for about 9.5 hours, for about 10 hours, for about 10.5 hours, for about 11 hours, for about 11.5 hours, for about 12 hours, for about 12.5 hours, for about 13 hours, for about 13.5 hours, for about 14 hours, for about 14.5 hours, for about 15 hours, for about 15.5 hours, for about 16 hours, for about 16.5 hours, for about 17 hours, for about 17.5 hours, for about 18 hours, for about 18.5 hours, for about 19 hours, for about 19.5 hours, for about 20 hours, for about 20.5 hours, for about 21 hours, for about 21.5 hours, for about 22 hours, for about 22.5 hours, for about 23 hours, for about 23.5 hours, for about 24 hours. In other embodiments, the cells are cultured at a heat shock temperature for about 1 hour to 24 hours daily over the span of 30 days.

In some embodiments, the cultured cells are in suspension, adherent, in suspension aggregates, or on suspension microcarriers.

In some embodiments, the cell density of the biomass increased upon differentiation.

### 5.1.1. Nucleic Acid Vector

In some embodiments, the vector construct used to transfect the avian, bovine, non-human mammalian, or non-human animal cells comprises: (a) a heat inducible heat shock promoter, and (b) a nucleic acid encoding one or more factors or biomarkers associated with (including that promote or indicate) a fat or muscle phenotype operably linked to the heat shock promoter. In other embodiments, the vector construct used to transfect the avian, bovine, non-human mammalian, or non-human animal cells comprises: (a) a heat inducible heat shock promoter, (b) a nucleic acid encoding one or more factors or biomarkers associated with (including that promote or indicate) a fat or muscle phenotype operably linked to the heat shock promoter, and (c) a nucleic acid encoding a heat shock factor, optionally operably linked to a heat shock promoter. The expression of the heat shock factor will promote and amplify the expression of the differentiation factor, biomarker, or factor associated with a fat or muscle phenotype.

In some embodiments, the vector construct used to transfect the avian, bovine, non-human mammalian, or non-human animal cells comprises: (a) a heat shock promoter derived from a non-human mammal and (b) a nucleic acid encoding PPARG, CEBPA, PAX7, MYOD, MYH2, and/or MEF2B operably linked to the heat shock promoter. In further embodiments, the expression of the nucleic acid is induced by a heat shock. In even further embodiments, the expression of the nucleic acid is higher in heat shock cells compared to non-heat shock cells. In other embodiments, the heat shock promoter is a HSP1A1 promoter or HSF-1 promoter. In additional embodiments, the heat shock promoter comprises the nucleotide sequence of SEQ ID NO: 29.

In some embodiments, the vector construct used to transfect the avian, bovine, non-human mammalian, or non-human animal cells comprises: (a) a heat shock promoter derived from a non-human mammal (b) a nucleic acid encoding PPARG, CEBPA, PAX7, MYOD, MYH2, and/or MEF2B operably linked to the heat shock promoter. In further embodiments, the expression of the nucleic acid is induced by a heat shock. In even further embodiments, the expression of the nucleic acid is higher in heat shock cells compared to non-heat shock cells. In other embodiments, the heat shock promoter is a HSP1A1 promoter or HSF-1 promoter. In additional embodiments, the heat shock promoter comprises the nucleotide sequence of SEQ ID NO: 29.

In some embodiments, the vector construct used to transfect the avian, bovine, non-human mammalian, or non-human animal cells comprises: (a) a heat shock promoter derived from a non-human mammal and (b) a nucleic acid encoding PPARG, CEBPA, PAX7, MYOD, MYH2, and/or MEF2B operably linked to the heat shock promoter. In further embodiments, the expression of the nucleic acid is induced by a heat shock. In even further embodiments, the expression of the nucleic acid is higher in heat shock cells compared to non-heat shock cells. In other embodiments, the heat shock promoter is a HSP1A1 promoter or HSF-1 promoter. In additional embodiments, the heat shock promoter comprises the nucleotide sequence of SEQ ID NO: 29.

In some embodiments, the vector construct used to transfect the avian, bovine, non-human mammalian, or non-human animal cells comprises: (a) a heat shock promoter derived from a non-human mammal and (b) a nucleic acid encoding MYH2 operably linked to the heat shock promoter. In other embodiments, the expression of MYH2 is induced in the absence of a heat shock event. In further embodiments, the expression of MYH2 is induced by a heat shock. In other embodiments, the heat shock promoter is a HSP1A1 promoter or HSF-1 promoter. In additional embodiments, the heat shock promoter comprises the nucleotide sequence of SEQ ID NO: 29.

In some embodiments, the nucleic acid encoding the factor or biomarker associated with a fat or muscle phenotype is incorporated into the host cell genome downstream or otherwise operably linked to an endogenous HSP promoter such that expression of the factor or biomarker associated with the fat or muscle phenotype is controlled by the endogenous HSP. Methods of engineering cells to insert a coding sequence downstream and operably linked to a targeted promoter are known in the art.

### 5.1.1.1. Vector

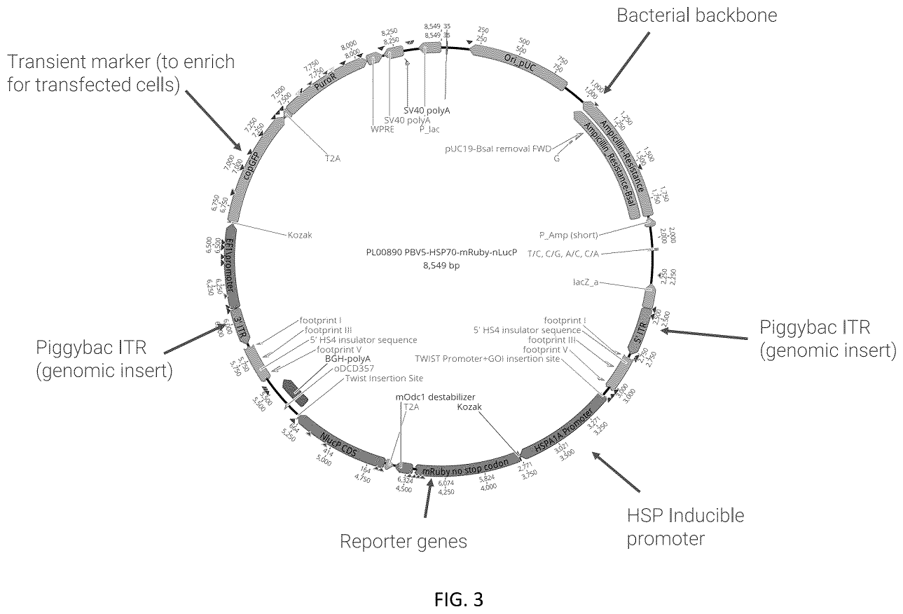

Provided herein are methods comprising transfecting the cells of the biomass with a vector construct comprising a nucleic acid encoding a differentiation factor, biomarker, or factor associated with a muscle or fat phenotype operably linked to a heat shock promoter. In some embodiments, the vector is a nucleic acid construct. In some embodiments, the vector can be selected from the group consisting of viral or non-viral vectors. In embodiments, the construct is that depicted in FIG. 3.

In some embodiments, the vector can be an expression vector. The term “expression vector” or “expression construct” includes any DNA vector, (e.g., a plasmid, cosmid, or phage chromosome) or construct that contains at least one promoter which is or may be operably linked to a downstream gene, coding region, or polynucleotide sequence to be transcribed (for example, a cDNA or genomic DNA fragment that encodes a protein, optionally, operably linked to sequence lying outside a coding region, an antisense RNA coding region, or RNA sequences lying outside a coding region). An expression vector or expression construct may also be a DNA construct comprising one or more promoters, wherein one or more of the promoters is not in fact operably linked to a polynucleotide sequence to be transcribed, but instead is designed for efficient insertion of an operably-linked polynucleotide sequence to be transcribed by the promoter. Transfection or transformation of the expression vector into a recipient cell allows the cell to express RNA encoded by the expression vector. An expression vector may be a genetically engineered plasmid, virus, transposon, or artificial chromosome derived from, for example, a bacteriophage, adenovirus, adeno-associated virus, retrovirus, or poxvirus. Such expression vectors can include sequences from bacteria, viruses or phages. Such vectors include chromosomal, episomal, and virus-derived vectors, for example, vectors derived from bacterial plasmids, bacteriophages, yeast episomes, yeast chromosomal elements, and viruses, vectors derived from combinations thereof, such as those derived from plasmid and bacteriophage genetic elements, cosmids, and phagemids. Thus, one exemplary vector is a double-stranded DNA phage vector. “Plasmid” and “vector” can be used interchangeably, as a plasmid is a commonly used form of vector. Moreover, the disclosed embodiments are intended to include other vectors which serve equivalent functions. Additional non-limiting examples of vector systems that can be used to integrate a nucleic acid sequence encoding one or more factors into the genome of the cells (e.g., the immortalized cell line) include: a sleeping beauty transposon system (as described in U.S. Pat. No. 7,985,739), a piggyBac transposition system (as described in US20090042297), CRISPR/Cas-mediated knock in, PhiC31 Integrase, Cre-Lox recombination, Lambda red recombination, TALENS/Zinc Fingers, random insertional mutagenesis, and viral vector-mediated integration. These vectors differ in many characteristics, such as their capacity to accommodate different sized nucleic acid inserts, their most efficient introduction method into the host cell and specifically in their utilization of the endogenous cell machineries of different types of host cells to ensure sufficient expression of the desired protein. A non-limiting example of a vector that can be used for the transfection described herein is shown in FIG. 3.

In embodiments, provided is a vector which comprises a nucleic acid encoding a single factor or biomarker associated with a fat or muscle phenotype, e.g. adipogenic or myogenic differentiation factor, biomarker, or other factor associated with a fat or muscle cell phenotype, or active fragment thereof, operably linked to a heat shock promoter. In embodiments, the vector comprises nucleic acids encoding two or more, three or more, or four or more, or 2, 3, 4, or more factors or biomarkers associated with a fat or muscle phenotype. In such cases, each of the 2, 3, 4, or more nucleic acid sequences are operably linked to a heat shock promoter and are processed from a polypeptide chain into the individual factors via a 2A self-cleaving polypeptide or are translated from a mRNA by IRES sequences to promote the expression of separate genes associated with a fat or muscle phenotype. For example, a nucleic acid is operably linked to a heterologous promoter when its transcription is under the control of the promoter. In another example, a nucleic acid encoding one or more factors or biomarkers associated with a fat or muscle phenotype can be linked to another nucleic acid encoding one or more factors or biomarkers associated with a fat or muscle phenotype by a nucleic acid encoding a self-cleaving 2A polypeptide or an internal ribosome entry site (IRES). In such cases, the promoter that is operably linked to the nucleic acids encoding the one or more factors or biomarkers associated with a fat or muscle phenotype controls expression of all of the nucleic acids encoding the one or more factors or biomarkers associated with a fat or muscle phenotype. In embodiments, the nucleic acids are on one vector and are operably linked either to a heat shock promoter upstream of the nucleic acids or operably linked to the other nucleic acids through a self-cleaving 2A polypeptide or an IRES.

In embodiments, a vector system is used to integrate a nucleic acid encoding one or more factors or biomarkers associated with a fat or muscle phenotype into the genome of the avian, bovine, non-human mammalian, or non-human animal cell, including for production of a cell line. In embodiments, the vector system is a PhiC31 Integrase Vector System. Additional non-limiting examples of vector systems that can be used to integrate a nucleic acid encoding one or more factors or biomarkers associated with a fat or muscle phenotype into the genome of the cells, including fibroblast or other appropriate cells, include: a sleeping beauty transposon system, a piggyBac transposition system, CRISPR/Cas-mediated knock in, Cre-Lox recombination, Lambda red recombination, TALENS/Zinc Fingers, random insertional mutagenesis, and viral vector-mediated integration. In embodiments, a transposon comprises the nucleic acids coding for PPARG, CEBPA, PAX7, MYOD, MYH2, and MEF2B, operably linked to a heat shock promoter. In embodiments, the vector system containing a transposon comprising the nucleic acids coding for PPARG, CEBPA, PAX7, MYOD, MYH2, and MEF2B, operably linked to a heat shock promoter, includes a Green Fluorescent Protein (GFP) marker. In embodiments, a vector system containing a transposase includes a Red Fluorescent Protein (RFP) marker. In embodiments, wherein the vector construct does not comprise an antibiotic resistance gene.

### 5.1.1.2. Heat Shock Promoter

The methods provided herein include transfecting cells with a nucleic acid encoding one or more factors or biomarkers associated with a fat or muscle phenotype operably linked to a heat shock promoter.

Heat shock proteins (HSPs) are a conserved protein family in prokaryotes and eukaryotes that protect cells from various stressors. HSPs play various roles, such as chaperoning other proteins, folding newly synthesized polypeptides, disassociating protein aggregates, protein complexing, and degrading misfolded proteins. The expression of these proteins is induced by environmental or/and physiological stressors, including but not limited to heat, heavy metals, ultraviolet light, hypoxia, and free radicals. Inducible heat shock promoters strongly regulate HSP gene expression.

The nucleic acids described herein can be engineered such that a heat shock promoter, or an inducible portion thereof, is operably linked to the nucleic acid encoding one or more factors or biomarkers associated with factors or biomarkers associated with a fat or muscle phenotype. The heat shock promoter can be any heat shock promoter that is inducible by exposure of the cell to temperatures that are higher than usually used for cell culture, which induce the activity of the heat shock promoter but do not significantly impact the viability of the cells. The heat shock promoter may be a portion, or combination of portions, of a naturally occurring heat shock promoter which inducibly promotes expression in response to exposure to heat shock temperatures. In some embodiments, the heat shock promoter is engineered for optimal target gene expression. In other embodiments, the heat shock promoter is of non-human origin. The heat shock promoter is, in embodiments, from the same species as the cells. In further embodiments, the heat shock promoter is of a different species than the origin of the cell line. In additional embodiment, the heat shock promoter is derived from animals, including but not limited to, cattle, pigs, sheep, goats, deer, rabbits, or poultry. In other embodiments, the heat shock promoter is a non-human HSP70 (HSPA1A) promoter, a non-human HSP60 promoter, a non-human HSP27 promoter, a non-human HSP100 promoter, a non-human HSP40 promoter, non-human a HSP110 promoter, a non-human HSF-1 promoter, or any other promoter regulated by increased temperature known in the art. In some embodiments, the heat shock promoter is of avian or bovine origin. In further embodiments, the heat shock promoter is a bovine heat shock promoter. In even further embodiments, the heat shock promoter is a Bos taurus heat shock promoter. In other embodiments, the heat shock promoter is a bovine HSPA1A or HSF-1 promoter. In other embodiments, the heat shock promoter is an avian heat shock promoter. In further embodiments, the heat shock promoter is a Gallus gallus heat shock promoter. In some embodiments, the heat shock promoter is an avian HSPA1A or HSF-1 promoter. In further embodiments, the bovine heat shock promoter is transfected into avian cells. In even further embodiments, the avian heat shock promoter is transfected into bovine cells. In embodiments, the heat shock promoter is a HSP70/HSPA1A promoter. In some embodiments, the heat shock promoter comprises the nucleotide sequence of SEQ ID NO: 29. In other embodiments, the heat shock promoter is an HSF-1 promoter. In further embodiments, the heat shock promoter contains heat shock elements that only respond to heat but not to other triggers such as heavy metals or UV. In other embodiments, the heat shock promoter is a portion of the heat shock promoter which has heat-inducible promoter activity.

### 5.1.1.3. Factors Promoting of a Fat or Muscle Phenotype

The methods provided herein include transfecting cells with a nucleic acid encoding one or more factors or biomarkers associated with a fat or muscle phenotype, e.g., a differentiation factor, a biomarker, or other encoded factor which promotes the fat or muscle phenotype, operably linked to a heat shock promoter. Also provided are cells and cell lines comprising the nucleic acid encoding one or more factors or biomarkers associated with a fat or muscle phenotype, e.g., a differentiation factor, biomarker, or other factor associated with a fat or muscle phenotype operably linked to the heat shock promoter or active portion thereof, and methods of producing cell based food products with cells comprising the nucleic acid vectors.

In some embodiments, one, two, three, four, or more factors or biomarkers associated with a fat or muscle phenotype are expressed are under the control of the same heat shock promoter. As disclosed herein, the multiple factors or biomarkers associated with a fat or muscle phenotype may be expressed from the same promoter to produce an mRNA transcript encoding the multiple genes associated with a fat or muscle phenotype, and either the individual factors or biomarkers associated with a fat or muscle phenotype are translated independently from the mRNA transcript from IRES sequences interspersed among the coding sequences or a polypeptide is translated from the mRNA transcript and then cleaved, for example, at 2A cleavage sequences, to process the polypeptide into the individual factors. In some embodiments, the genes associated with a fat or muscle phenotype are derived from the same species as the cells comprising the nucleic acid encoding the differentiation factor, i.e. a CIS gene.

Factors or biomarkers associated with fat phenotype, e.g. adipogenic differentiation factors or biomarkers, may activate adipogenesis, which is a process of sequential changes in the expression of specific genes that facilitate the formation of cells having phenotypes characteristic of mature adipocytes. Precursor cells, including but not limited to fibroblasts, can express phenotypes associated with fat or muscle phenotypes, e.g., differentiate, upon expression of genes associated with fat or muscle phenotypes, e.g., differentiation factors. Factors or biomarkers associated with fat phenotype, e.g. adipogenic differentiation factors, include, but aren't limited to, CEBPA, PPARG, KLFs, SREBP, IRS1, and GLUT4. In some embodiments, the factor is PPARG or CEBPA. In further embodiments, PPARG and CEBPA are co-expressed under the control of a heat shock promoter. In other embodiments, biomarkers associated with the fat phenotype include but are not limited to LEP, LPL, SCD1, or other fat phenotype biomarkers known in the art.

Myogenesis is the formation of skeletal muscular tissue. Muscle fibers generally form through the fusion of precursor myoblasts into multinucleated fibers called myotubes. Factors or biomarkers associated with a muscle phenotype, e.g. myogenic differentiation factors, include but are not limited to MYOD, Myf5, myogenin, MRF4, PAX7, MERFs, or other myogenic differentiation factors known in the art. In some embodiments, the factors are PAX7, MYOD, and/or MEF2B (MERFs). In further embodiments, PAX7, MYOD, and/or MEF2B are co-expressed under the control of a heat shock promoter. In even further embodiments, MEF2B and MYOD are co-expressed under the control of a heat shock promoter. In embodiments, biomarkers associated with the muscle phenotype include but are not limited to MYH2, TNNT, MSTN, CK-M or other muscle phenotype biomarkers known in the art. In some embodiments, MYH2 is expressed under the control of a heat shock promoter. In further embodiments, MYH2 is expressed with or without heat shock mediated induction. Provided are constructs in which a nucleotide sequence encoding MYH2 is operably linked to a heat shock promoter. Alternatively, factors or biomarkers associated with characteristic muscle proteins, e.g. myosin, may be directly or indirectly expressed. An increased expression of such muscle proteins may result in improved textural properties for the cell.

In embodiments, the expression of one or more factors or biomarkers associated with a fat or muscle phenotype is higher in heat shocked cells than non-transfected cells or cells cultured at a standard temperature for a time. In further embodiments, the expression of one or more biomarkers associated with a fat or muscle phenotype is substantially higher in heat shocked cells than non-transfected cells or cells cultured at a standard temperature for a time. In some embodiments, the expression of one or more biomarkers associated with a fat or muscle phenotype is at 0.5 to 30 fold higher in heat shocked cells than non-transfected cells or cells cultured at a standard temperature for a time. In other embodiments, the expression of the expression of one or more biomarkers associated with a fat or muscle phenotype is 0.5 fold, 1 fold, 2 fold, 3 fold, 4 fold, 5 fold, 6 fold, 7 fold, 8 fold, 9 fold, 10 fold, 11 fold, 12 fold, 13 fold, 14 fold, 15 fold, 16 fold, 17 fold, 18 fold, 19 fold, 20 fold, 21 fold, 22 fold, 23 fold, 24 fold, 25 fold, 26 fold, 27 fold, 28 fold, 29 fold or 30 fold higher in heat shocked cells than non-transfected cells or cells cultured at a standard temperature for a time.

### 5.1.1.3.1. PPARG

In embodiments, a cell line is transfected with a nucleic acid sequence encoding a PPARG polypeptide, or a fragment thereof, operably linked to a heat shock promoter, for use in the methods and cell lines disclosed herein. As used herein, “PPAR-7” refers to peroxisome proliferator activated receptor gamma gene or PPAR-7 polypeptide that is a member of the PPAR subfamily of nuclear receptors. PPAR-7 protein regulates fatty acid storage and glucose metabolism. In embodiments, PPARG refers to a PPARG gene or PPAR-7 polypeptide, or a variant thereof (e.g., a PPAR-7 polypeptide having one or more (e.g., one, two, three, four, five, six, seven, eight, nine, ten, or more amino acid substitutions, deletions or insertions as compared to a wild type PPAR-7 polypeptide)).

In embodiments referring to a PPAR-7 polypeptide, the amino acid sequence of the PPAR-7 polypeptide is at least 80% (e.g., at least 85%, 90, 95%, 96%, 97%, 98%, or 99%) identical to SEQ ID NOs: 19-24.

### 5.1.1.3.2. CEBPA

In embodiments, a cell line is transfected with a nucleic acid sequence encoding a CEBPA polypeptide, or a fragment thereof, operably linked to a heat shock promoter, for use in the methods and cell lines disclosed herein. As used herein, “CEBPα” refers to CCAAT/enhancer binding protein alpha gene or CEBPA, which is a transcription factor crucial for regulating proliferation, cellular differentiation, and metabolism. In embodiments, CEBPA refers to a CEBPA gene or CEBPα polypeptide, or a variant thereof (e.g., a CEBPα polypeptide having one or more (e.g., one, two, three, four, five, six, seven, eight, nine, ten, or more amino acid substitutions, deletions or insertions as compared to a wild type CEBPα polypeptide)).

In embodiments referring to a CEBPA polypeptide, the amino acid sequence of the PPAR-γ polypeptide is at least 80% (e.g., at least 85%, 90, 95%, 96%, 97%, 98%, or 99%) identical to SEQ ID NO: 31.

### 5.1.1.3.3. PAX7

In embodiments, a cell line is transfected with a nucleic acid encoding a PAX7 polypeptide, or a fragment thereof, operably linked to a heat shock promoter, for use in the methods and cell lines disclosed herein. As used herein, “PAX7” refers to paired box 7 (PAX7) gene or PAX7 polypeptide that is a member of the paired box (PAX) family of transcription factors that typically contain a paired box domain, an octapeptide, and a paired-type homeodomain. These genes play critical roles during muscle development. In embodiments, PAX7 refers to a PAX7 gene or PAX7 polypeptide, or a variant thereof (e.g., a PAX7 polypeptide having one or more (e.g., one, two, three, four, five, six, seven, eight, nine, ten, or more amino acid substitutions, deletions, or insertions as compared to a wild type PAX7 polypeptide)).

In embodiments, referring to a PAX7 polypeptide, the amino acid sequence of the PAX7 polypeptide is at least 80% (e.g., at least 85%, 90, 95%, 96%, 97%, 98%, or 99%) identical to SEQ ID NOs: 7-12.

### 5.1.1.3.4. MYOD

In embodiments, a cell line is transfected with a nucleic acid sequence encoding a MYOD polypeptide, or a fragment thereof, operably linked to a heat shock promoter, for use in the methods and cell lines disclosed herein. As used herein, “MYOD” refers to the myogenic differentiation 1 (MYOD1) gene or MYOD or MYDO1 protein that is a nuclear protein that belongs to the basic helix-loop-helix family of transcription factors and the myogenic factors subfamily. MYOD regulates muscle cell differentiation and muscle regeneration. MYOD activates its own transcription which may stabilize commitment to myogenesis, and acts as a transcriptional activator that promotes transcription of muscle-specific target genes. In embodiments, MYOD refers to the MYOD1 gene or MYOD or MYODI polypeptide, or a variant thereof (e.g., a MYOD polypeptide having one or more (e.g., one, two, three, four, five, six, seven, eight, nine, ten, or more) amino acid substitutions, deletions, or insertions as compared to a wild type MYOD polypeptide). Non-limiting examples of MYOD polypeptides are described in Table 1.

In embodiments referring to a MYOD polypeptide, the amino acid sequence of the MYOD polypeptide is at least 80% (e.g., at least 85%, 90, 95%, 96%, 97%, 98%, or 99%) identical to SEQ ID NOs: 1-6.

### 5.1.1.3.5. MEF2B

In embodiments, a cell line is transfected with a nucleic acid sequence encoding a MEF2B polypeptide, or a fragment thereof, operably linked to a heat shock promoter, for use in the methods and cell lines disclosed herein. As used herein, “MEF2B” refers to myocyte enhancer factor 2B (MEF2B) gene or MEF2B polypeptide that is a member of the MADS/MEF2 family of DNA binding proteins. MEF2B protein regulates gene expression, including expression of the smooth muscle myosin gene. In embodiments, MEF2B refers to a MEF2B gene or MEF2B polypeptide, or a variant thereof (e.g., a MEF2B polypeptide having one or more (e.g., one, two, three, four, five, six, seven, eight, nine, ten, or more amino acid substitutions, deletions or insertions as compared to a wild type MEF2B polypeptide)).

In embodiments referring to a MEF2B polypeptide, the amino acid sequence of the MEF2B polypeptide is at least 80% (e.g., at least 85%, 90, 95%, 96%, 97%, 98%, or 99%) identical to SEQ ID NOs: 13-18.

### 5.1.1.3.6. MYH2

In embodiments, a cell line is transfected with a nucleic acid sequence encoding a myosin heavy chain polypeptide, or a fragment thereof, operably linked to a heat shock promoter, for use in the methods and cell lines disclosed herein. As used herein, “MYH2” refers to myosin heavy chain 2A (MYH2) gene or MYH2 polypeptide is an actin-based motor molecule with ATPase activity essential for muscle contraction. In embodiments, MYH2 refers to a MYH2 gene or MYH2 polypeptide, or a variant thereof (e.g., a MYH2 polypeptide having one or more (e.g., one, two, three, four, five, six, seven, eight, nine, ten, or more amino acid substitutions, deletions or insertions as compared to a wild type MYH2 polypeptide)). In embodiments referring to a MYH2 polypeptide, the amino acid sequence of the MYH2 polypeptide is at least 80% (e.g., at least 85%, 90, 95%, 96%, 97%, 98%, or 99%) identical to SEQ ID NOs: 26 and 28. In embodiments, the avian, bovine, non-human mammalian, or non-human animal cells are transfected with a vector construct comprising: (a) a HSP1A1 promoter or a HSF-1 promoter and (b) a nucleic acid encoding MYHC having the nucleotide sequence of SEQ ID NO: 25 or SEQ ID NO: 27 operably linked to the HSP1A1 or HSF-1 promoter. In other embodiments, MYHC expression is induced with or without a heat shock induction.

### 5.1.1.4. Heat Shock Factor

The methods provided herein further include transfecting cells with a nucleic acid encoding a one or more genes associated with a fat or muscle phenotype operably linked to a heat shock promoter, and a nucleic acid encoding a heat shock factor also operably linked to a heat shock promoter or linked to one or more genes associated with a fat or muscle phenotype.

Heat shock factors (HSFs) are transcription factors that regulate the expression of HSPs inside cellular systems upon stress. Dual expression of the one or more genes associated with a fat or muscle phenotype and a heat shock factor by the inducible heat shock promoter enables a positive feedback loop, wherein the continuously synthesized heat shock factor continuously activates the heat shock promoter which then continuously activates expression of the differentiation factor when cells are cultured at a heat shock temperature.

In some embodiments, the heat shock factor is heat shock factor 1 (HSF-1).

Exemplary amino acid sequences of the differentiation factors and heat shock factors used in the methods and cell lines described herein are provided in Table 1.

TABLE 1

| | | NCBI | NCBI | |
|---|---|---|---|---|
| Gene | | Protein | mRNA Ref. | |
| Symbol | Species | Ref. No. | No. | Sequence |
| MYOD | Gallus | NP_989545.3 | NM_204214.3 | MDLLGPMEMTEGSLCSFTAADD |
| | (chicken) | (SEQ ID NO: 1) | (encodes | FYDDPCFNTSDMHFFEDLDPRL |
| | | | NP_989545.3) | VHVGGLLKPEEHPHHHGHHHG |
| | | | | NPHEEEHVRAPSGHHQAGRCLL |
| | | | | WACKACKRKTTNADRRKAATM |
| | | | | RERRRLSKVNEAFETLKRCTSTN |
| | | | | PNQRLPKVEILRNAIRYIESLQAL |
| | | | | LREQEDAYYPVLEHYSGESDAS |
| | | | | SPRSNCSDGMMEYSGPPCSSRRR |
| | | | | NSYDSSYYTESPNDPKHGKSSV |
| | | | | VSSLDCLSSIVERISTDNSTCPILP |
| | | | | PAEAVAEGSPCSPQEGANLSDSG |
| | | | | AQIPSPTNCTPLPQESSSSSSSNPI |
| | | | | YQVL (SEQ ID NO: 1) |
| MYOD | Anas | NP_001297287.1 | NM_001310358.1 | MDLLGAMEMPEGSLCSFTGADD |
| | platyrhynchos | (SEQ ID NO: 2) | (encodes | FYDDPCFNTSDMHFFEDLDPRL |
| | (Duck) | | NP_001297287.1) | VHVGGLLKPEEHPHHHGHHHH |
| | | | | GHPHEEEHVRAPSGHHQAGRCL |
| | | | | LWACKACKRKTTNADRRKAAT |
| | | | | MRERRRLSKVNEAFETLKRCTS |
| | | | | TNPNQRLPKVEILRNAIRYIESLQ |
| | | | | ALLREQEDVYYPVLEHYSGESD |
| | | | | ASSPRSNCSDGMMEYSGPPCSSR |
| | | | | RRNSYDSSYYTESPNDPKHGKSS |
| | | | | VVSSLDCLSGIVERISTDNSTCPI |
| | | | | LPPAETVAEGSPCSPQEGASLND |
| | | | | SGAQIPSPTNCTPLPQESSSSNPIY |
| | | | | QVL (SEQ ID NO: 2) |
| MYOD | Meleagris | NP_001290100.1 | NM_001303171.1 | MDLLGPMEMTEGSLCSFAAADD |
| | gallopavo | (SEQ ID NO: 3) | (encodes | FYDDPCFNTSDMHFFEDLDPRL |
| | (Turkey) | | NP_001290100.1) | VHVGGLLKPEEHPHHHGHHHG |
| | | | | HPHEEEHVRAPSGHHQAGRCLL |
| | | | | WACKACKRKTTNADRRKAATM |
| | | | | RERRRLSKVNEAFETLKRCTSTN |
| | | | | PNQRLPKVEILRNAIRYIGSLQAL |
| | | | | LREQEDAYYPVLEHYSGESDAS |
| | | | | SPRSNCSDGMMEYSGPPCSSRRR |
| | | | | NSYDSSYYTESPNDPKHGKSSV |
| | | | | VSSLDCLSSIVERISTDNSTCPILP |
| | | | | PAEAVAEGSPCSPQEGASLNDSG |
| | | | | AQIPSPTNCTPLPQDSSSSNNPIY |
| | | | | QVL (SEQ ID NO: 3) |
| MYOD | Sus scrofa | NP_001002824.1 | NM_001002824.1 | MELLSPPLRDVDLTGPDGSLCNF |
| | (Porcine) | (SEQ ID NO: 4) | (encodes | ATADDFYDDPCFDSPDLRFFEDL |
| | | | NP_001002824.1) | DPRLVHVGALLKPEEHSHFPAA |
| | | | | A |
| | | | | HPAPGAREDEHVRAPSGHHQAG |
| | | | | RCLLWACKACKRKTTNADRRK |
| | | | | AATMRERRRLSKVNEAFETLKR |
| | | | | CTSSNPNQRLPKVEILRNAIRYIE |
| | | | | GLQALLRDQDAAPPGAAAAFYA |
| | | | | PGPLPPGRGGEHYSGDSDASSPR |
| | | | | SNCSDGMMDYSGPPSGARRRNC |
| | | | | YDGTYYSEAPSEPRPGKNAAVS |
| | | | | SLDCLSSIVESISTESPAAPALLLA |
| | | | | DTPRESSPGPQEAAAGSEVERGT |
| | | | | PTPSPDAAPQCPASANPNPIYQV |
| | | | | L (SEQ ID NO: 4) |
| MYOD | Bos taurus | NP_001035568.2 | NM_001040478.2 | MELLSPPLRDVDLTGPDGSLCNF |
| | (Bovine) | (SEQ ID NO: 5) | (encodes | ATADDFYDDPCFDSPDLRFFEDL |
| | | | NP_001035568.2) | DPRLVHVGALLKPEEHSHFPAA |
| | | | | AHPAPGAREDEHVRAPSGHHQA |
| | | | | GRCLLWACKACKRKTTNADRR |
| | | | | KAATMRERRRLSKVNEAFETLK |
| | | | | RCTSSNPNQRLPKVEILRNAIRYI |
| | | | | EGLQALLRDQDAAPPGAAAAFY |
| | | | | APGPLPPGRSGEHYSGDSDASSP |
| | | | | RSNCSDGMMDYSGPPSGARRRN |
| | | | | CYDRTYYSEAPNEPRPGKSAAV |
| | | | | SSLDCLSSIVERISTESPAAPALLL |
| | | | | ADAPPESSPGPQEAAGSEVERGT |
| | | | | PAPSPDTAPQGLAGANPNPIYQV |
| | | | | L (SEQ ID NO: 5) |
| MYOD | Oreochromis | NP_001266649.1 | NM_001279720.1 | MELSDISFPIPTADDFYDDPCENT |
| | niloticus | (SEQ ID NO: 6) | (encodes | SDMHFFEDLDPRLVHVGLLKPD |
| | (Tilapia) | | NP_001266649.1) | DSSSSSSSSPSSSSSSPSSLLHLHH |
| | | | | HAEVEDDEHVRAPSGHHQAGR |
| | | | | CLLWACKACKRKTTNADRRKA |
| | | | | ATLRERRRLSKVNDAFETLKRC |
| | | | | TTANPNQRLPKVEILRNAISYIES |
| | | | | LQALLRGGQEDGFYPVLEHYSG |
| | | | | DSDASSPRSNCSDGMTDENGPT |
| | | | | CQTTRRGSYDSSSYFSETPNGGL |
| | | | | KSERSSVVSSLDCLSSIVERISTD |
| | | | | NSSLLPPADGPGSPTTTTTVPVG |
| | | | | EAGTAPATAQVSSPTASQDPNLI |
| | | | | YQVL (SEQ ID NO: 6) |
| PAX7 | Gallus | NP_990396.1 | NM_205065.1 | MAALPGTVPRMMRPAPGQNYP |
| | (chicken) | (SEQ ID NO: 7) | (encodes | RTGFPLEVSTPLGQGRVNQLGG |
| | | | NP_990396.1) | VFINGRPLPNHIRHKIVEMAHHG |
| | | | | IRPCVISRQLRVSHGCVSKILCRY |
| | | | | QETGSIRPGAIGGSKPRQVATPD |
| | | | | VEKKIEEYKRENPGMFSWEIRDR |
| | | | | LLKDGHCDRSTVPS |
| | | | | VSSISRVLRIKFGKKEEEEDCDK |
| | | | | KEEDGEKKAKHSIDGILGDKGN |
| | | | | RLDEGSDVESEPDLPLKRKQRRS |
| | | | | RTTFTAEQLEELEKAFERTHYPD |
| | | | | IYTREELAQRTKLTEARVQVWF |
| | | | | SNRRARWRKQAGANQLAAFNH |
| | | | | LLPGGFPPTGMPTLPP |
| | | | | YQLPDSTYPTTTISQDGGSTVHR |
| | | | | PQPLPPSTMHQGGLAAAAAADS |
| | | | | SSAYGARHSFSSYSDSFMNAAA |
| | | | | PANHMNPVSNGLSPQKOGAQN |
| | | | | KMQCSRWNLTIALNNQVMSILS |
| | | | | NPSGVPPQPQADFSISPLHGGLD |
| | | | | TTNSISASCSQRSDSIKSVDSLPT |
| | | | | SQSYCPPTYSTTSYSVDPVAGYQ |
| | | | | YGQYGQTAVDYLTKNVSLSTQR |
| | | | | RMKLGEHSAVLGLLPVETGQAY |
| | | | | (SEQ ID NO: 7) |
| PAX7 | Anas | NP_001297324.1 | NM_001310395.1 | MAALPGTVPRMMRPAPGQNYP |
| | platyrhynchos | (SEQ ID NO: 8) | (encodes | RTGFPLEVSTPLGQGRVNQLGG |
| | (Duck) | | NP_001297324.1) | VFINGRPLPNHIRHKIVEMAHHG |
| | | | | IRPCVISRQLRVSHGCVSKILCRY |
| | | | | QETGSIRPGAIGGSKPRVATPDV |
| | | | | EKKIEEYKRENPGMFSWEIRDRL |
| | | | | LKDGHCDRSTVPSGLVSSISRVL |
| | | | | RIKFGKKEEEEDCDKKEEDGEK |
| | | | | KAKHSIDGILGDKGNRLDEGSD |
| | | | | VESEPDLPLKRKQRRSRTTFTAE |
| | | | | QLEELEKAFERTHYPDIYTREEL |
| | | | | AQRTKLTEARVQVWFSNRRAR |
| | | | | WRKQAGANQLAAFNHLLPGGF |
| | | | | PPTGMPTLPPYQLPDSTYPTTTIS |
| | | | | QDGGSTVHRPQPLPPSTMHQGG |
| | | | | LAAAAAADTGSAYGARHSFSSY |
| | | | | SDSFMNAAAPANHMNPVSNGLS |
| | | | | PQVMSILSNPSGVPPQPQADFSIS |
| | | | | PLHGGLDTTNSISASCSQRSDSIK |
| | | | | SVDSLPTSQSYCPPTYSTTSYSV |
| | | | | DPVAGYQYGQYGQTAVDYLAK |
| | | | | NVSLSTQRRMKLGGHSAVLGLL |
| | | | | PVETGQAY (SEQ ID NO: 8) |
| PAX7 | Meleagris | NP_001290114.1 | NM_001303185.1 | MAALPGTVPRMMRPAPGQNYP |
| | gallopavo | (SEQ ID NO: 9) | (encodes | RTGFPLEVSTPLGQGRVNQLGG |
| | (Turkey) | | NP_001290114.1) | VFINGRPLPNHIRHKIVEMAHHG |
| | | | | IRPCVISRQLRVSHGCVSKILCRY |
| | | | | QETGSIRPGAIGGSKPRQVATPD |
| | | | | VEKKIEEYKRENPGMFSWEIRDR |
| | | | | LLKDGHCDRSTVPSVSSISRVLRI |
| | | | | KFGKKEEEEDCDKKEEDGEKKA |
| | | | | KHSIDGILGDKGNRLDEGSDVES |
| | | | | EPDLPLKRKQRRSRTTFTAEQLE |
| | | | | ELEKAFERTHYPDIYTREELEKA |
| | | | | FERTHYPDIYTREELAQRTKLTE |
| | | | | ARVQVWFSNRRARWRKQAGAN |
| | | | | QLAAFNHLLPGGFPPTGMPTLPP |
| | | | | YQLPDS |
| | | | | TYPTTTISQDGSSTVHRPQPLPPS |
| | | | | TMHQGGLAAAAAADSSSAYGA |
| | | | | RHSFSSYSDSFMNAAAPANHMN |
| | | | | PVSNGLSPQVMSILSNPSGVPPQ |
| | | | | PQADFSISPLHGGLDTTNSISASC |
| | | | | SQRSDSIKSVDSLPTSQSYCPPTY |
| | | | | STTSYSVDPVAGYQYGQYGQTA |
| | | | | VDYLAKNVSLSTQRRMKLGEHS |
| | | | | AVLGLLPVETGQAY (SEQ ID |
| | | | | NO: 9) |
| PAX7 | Sus scrofa | XP_020951119.1 | XM_021095460.1 | MAALPGTVPRMMRPAPGQNYP |
| | (Porcine) | (SEQ ID NO: 10) | (encodes | RTGFPLEVSTPLGQGRVNQLGG |
| | | | XP_020951119.1) | VFINGRPLPNHIRHKIVEMAHHG |
| | | | | IRPCVISRQLRVSHGCVSKILCRY |
| | | | | QETGSIRPGAIGGSKPRQVATPD |
| | | | | VEKKIEEYKRENPGMFSWEIRDR |
| | | | | LLKDGHCDRSTVPSVSSISRVLRI |
| | | | | KFGKKEEDDEADKKEDDSEKKA |
| | | | | KHSIDGILGDKGNRLDEGSDVES |
| | | | | EPDLPLKRKQRRSRTTFTAEQLE |
| | | | | ELEKAFERTHYPDIYTREELAQR |
| | | | | TKLTEARVQVWFSNRRARWRK |
| | | | | QAGANQLAAFNHLLPGGFPPTG |
| | | | | MPTLPPYQLPDSTYPTTTISQDG |
| | | | | GSTVHRP |
| | | | | QPLPPSTMHQGGLAAAAAAADT |
| | | | | SSAYGARHSFSSYSDSFMNPAAP |
| | | | | SNHMNPVSNGLSPQVMSILSNPS |
| | | | | AVPPQPQADFSISPLHGGLDSAT |
| | | | | SISASCSQRADSIKPGDSLPTSQS |
| | | | | YCPPTYSTTGYSVDPVAGYQYG |
| | | | | QYGQTAVDYLAKNVSLSTQRR |
| | | | | MKLGEHSAVLGLLPVETGQAY |
| | | | | (SEQ ID NO: 10) |
| PAX7 | Bos taurus | XP_015316176.1 | XM_015460690.2 | MAALPGTVPRMVRPAPGQNYPR |
| | (Bovine) | (SEQ ID NO: 11) | (encodes | TGFPLEVSTPLGQGRVNQLGGV |
| | | | XP_015316176.1) | FINGRPLPNHIRHKIVEMAHHGI |
| | | | | RPCVISRQLRVSHGCVSKILCRY |
| | | | | QETGSIRPGAIGGSKPRQVATPD |
| | | | | VEKKIEEYKRENPGMFSWEIRDR |
| | | | | LLKDGHCDRSTVPSVSSISRVLRI |
| | | | | KFGKKEEEDEADKKEEDGEKKA |
| | | | | KHSIDGILGDKGNRLDEGSDVES |
| | | | | EPDLPLKRKQRRSRTTFTAEQLE |
| | | | | ELEKAFERTHYPDIYTREELAQR |
| | | | | TKLTEARVQVWFSNRRARWRK |
| | | | | QAGANQLAAFNHLLPGGFPPTG |
| | | | | MPTLPPYQLPDSTYPTTTISQDG |
| | | | | GSTVHRPQPLPPSTMHOGGLAA |
| | | | | AAAADTSSAYGARHGFSSYSDS |
| | | | | FMNPAAASNHMNPVSNGLSPQ |
| | | | | WSLTVLFGVQGHHLHLIGAED |
| | | | | (SEQ ID NO: 11) |
| PAX7 | Oreochromis | XP_025763203.1 | XP_025907418.1 | MATLPGTVPRMVRPAPGQNYPR |
| | niloticus | (SEQ ID NO: 12) | (encodes | TGFPLEVSTPLGQGRVNQLGGV |
| | (Tilapia) | | XP_025763203.1) | FINGRPLPNHIRHKIVEMAHHGI |
| | | | | RPCVISRQLRVSHGCVSKILCRY |
| | | | | QETGSIRPGAIGGSKPRQVATPD |
| | | | | VEKRIEEYKRENPGMFSWEIRDK |
| | | | | LLKDGVCDRSTVPSGNRTDDAS |
| | | | | DVDSEPDLPLKRKQRRSRTTFTA |
| | | | | EQLEELEKAFERTHYPDIYTREE |
| | | | | LAQRTKLTEARVQVWFSNRRAR |
| | | | | WRKQAGANQLAAFNHLLPGGF |
| | | | | PPTGMPTLPTYQLPESSYPSTTLS |
| | | | | QEGSSTLHRPQPLPPSSMHQGGL |
| | | | | SADSSSAYGLSSNRHSFSSYSDT |
| | | | | FMSPSASSNHMNPVGNGLSPQV |
| | | | | MSILSNPSAVPSQPQHDFSISPLH |
| | | | | SSLESSNPISASCSQRSDTIKSVDS |
| | | | | LASSQSYCPPTYSATSYSVDPVT |
| | | | | AGYQYSQYGQTAVDYLAKNVS |
| | | | | LSTQRRMKLGDHSAVLGLLQVE |
| | | | | TGQAY (SEQ ID NO: 12) |
| MEF2B | Gallus | XP_430389.3, | XM_430389.7 | MGRKKIQISRILDQRNRQVTFTK |
| | (chicken) | isoform X1 | (encodes | RKFGLMKKAYELSVLCDCEIALI |
| | | (SEQ ID NO: 13) | XP_430389.3) | IFNSTNRLFQYASTDMDKVLLK |
| | | | | YTEYSEPHESRTNSDILETLKRK |
| | | | | GLGLESHELELEEGLDPGEKLRQ |
| | | | | MNEGMDLTVARPRFYSPVPLPE |
| | | | | VPYGSSPSSSSDGALGSASSSPQS |
| | | | | QGRPPAFKPTAPKPSGRSPGPMP |
| | | | | PGIGYPLFPAGSLNRALATKTPPP |
| | | | | LYLGADGQRRSGGSTARPLYSG |
| | | | | LQTLNPVLASGSASIPSHSLTGFP |
| | | | | FLAPAQAAEFGAGEAPPPPGFLQ |
| | | | | PGPPAPWQPPRDMAALGASSRI |
| | | | | VPTEDLAPGSSPQPHAISIKSERV |
| | | | | SPGLGCPSGAPQPSQGLTSLSEA |
| | | | | PQGAADLQPRDDYSKGYPYPPP |
| | | | | PPRPLAEEQRATAAVPVPARRA |
| | | | | QAEDAWQR (SEQ ID NO: 13) |
| MEF2B | Anas | XP_038025156.1 | XM_038169228.1 | MGRKKIQISRILDQRNRQVTFTK |
| | platyrhynchos | (SEQ ID NO: 14) | (encodes | RKFGLMKKAYELSVLCDCEIALI |
| | (Duck) | | XP_038025156.1) | IFNSTNRLFQYASTDMDKVLLK |
| | | | | YTEYSEPHESRTNSDILETLKRK |
| | | | | GLGLDSHELELDEGLDGGEKMR |
| | | | | KLNEGMDLTVARPRFYSPVPLPE |
| | | | | ASYSSSPPAGGDGALGSTSSSPQ |
| | | | | SQGRPPAFKPSAPKLSGRSPGPM |
| | | | | PPGIGYPLFPPGSLNRALATKTPP |
| | | | | PLYLGADGRRGEAHGSLASGRS |
| | | | | GGSAARPLYPGLQTLSPVLPPGS |
| | | | | AGIPNHSLSGFPFLAPAQAEYGA |
| | | | | GEAPPPPGFLQPGPPASWQPPRD |
| | | | | MAALGTSTRIVPAEDAAPGSSPQ |
| | | | | HHAISIKSERVSPGLGCPSGTPQP |
| | | | | PPGGLTSLNEAPRGSGDLQPRDD |
| | | | | YAKGYPYPLGPPRPLAEEQRATT |
| | | | | VPVPPRRAQAVDGWQR (SEQ ID |
| | | | | NO: 14) |
| MEF2B | Meleagris | XP_010723330.1 | M_010725028.3 | MGRKKIQISRILDQRNRQVTFTK |
| | gallopavo | (SEQ ID NO: 15) | (encodes | RKFGLMKKAYELSVLCDCEIALI |
| | (Turkey) | | XP_010723330.1) | IFNSTNRLFQYASTDMDKVLLK |
| | | | | YTEYSEPHESRTNSDILETLKRK |
| | | | | GLGLESHELELEEGLDPGEKLRQ |
| | | | | LNEGMDLTVARPRFYSPVPLPEV |
| | | | | PYGSSPPSSSDGALGSASSSPQSQ |
| | | | | GRPPTFKPTAPKPSGRSPGPMPP |
| | | | | GIGYPLFPAGSLNRALATKTPPP |
| | | | | LYLGADGQRRSGSGAARPLYSG |
| | | | | LQTLSPVLASGSAGIPSHSLTGFP |
| | | | | FLTPAQAEFGAGEAPPPPGFLQP |
| | | | | GPPAPWQPPRDMAALGASSRIIP |
| | | | | TEDLAPGSSPQPHTISIKSERVSP |
| | | | | GLGCPSGTPQPSPGSLTSLSEAPR |
| | | | | GAADLQPRDDYTKGYPYPLPPP |
| | | | | RPLAEEQRATATIPVPARRVQAE |
| | | | | DAWQR (SEQ ID NO: 15) |
| MEF2B | Sus scrofa | XP_020939100.1 | XM_021083441.1 | MPRPISSRGAGPPALKAPVAAGS |
| | (Porcine) | (SEQ ID NO: 16) | (encodes | SEFCAATAAARCVRERQSECPG |
| | | | XP_020939100.1) | GADQGYSTQPGTMGRKKIQISRI |
| | | | | LDQRNRQVTFTKRKFGLMKKA |
| | | | | YELSVLCDCEIALIIFNSANRLFQ |
| | | | | YASTDMDRVLLKYTEYSEPHES |
| | | | | RTNTDILETLKRRGVGLDGPELE |
| | | | | PDEGLEGPGEKLRRLAGDGGDP |
| | | | | ALPRPRLYPAAPTMPSPDMVYG |
| | | | | ALPPPGCDPSGLGEALPAQSRPS |
| | | | | PFRPAAPKAGPPGLAHPLFSPSH |
| | | | | LASKTPPPLYLATDGRRPDLPGG |
| | | | | LAGARGGLSTSRGLYGSLQSPCS |
| | | | | TATPGTPLGSFPFLPAGPPEYGL |
| | | | | GDPPPPPGLLQPPTLAPWQPSRG |
| | | | | DGPTATPTQSSGGRSLGEEGPPA |
| | | | | RGASSPTPPVSIKSERLSPAPGGP |
| | | | | GDFPKTFPYPLLLARPLAEPLRP |
| | | | | GPPMRRLPTADGWPR (SEQ ID |
| | | | | NO: 16) |
| MEF2B | Bos taurus | NP_001139265.1 | NM_001145793.1 | MGRKKIQISRILDQRNRQVTFTK |
| | (Bovine) | (SEQ ID NO: 17) | (encodes | RKFGLMKKAYELSVLCDCEIALI |
| | | | NP_001139265) | IFNSSNRLFQYASTDMDRVLLKY |
| | | | | TEYSEPHESRTITDILETLKRRGV |
| | | | | GLDGPELEPDEGLEGPGEKLRRL |
| | | | | AGDGGDPALPRPRLYPAAPTMP |
| | | | | SPDMVYGALPPPACEPTGLGEA |
| | | | | LPAQSRPSPFRPAAPKAGPPGLA |
| | | | | HPLFSPSHLASKTPPPLYLAADG |
| | | | | RRPDLPGGLAGTRGGLSSSRGLY |
| | | | | GGLQSPCSTAAPGPPLGSFPFLPA |
| | | | | GPPEYGLGDPPPPPGLLQPPTLAP |
| | | | | WQPSRADGPPATPTQPSGGRSL |
| | | | | GEDGPPARGASSPTPPVSIKSERL |
| | | | | SPAPGGPGDFPKTFPYPLLLARP |
| | | | | LAEPLRPGPPLRRLPTADGWPR |
| | | | | (SEQ ID NO: 17) |
| MEF2B | Oreochromis | XP_005479045.1 | XM_005478988.4 | MGRKKIQISRILDQRNRQVTFTK |
| | niloticus | (SEQ ID NO: 18) | (encodes | RKFGLMKKAYELSVLCDCEIALI |
| | (Tilapia) | | XP_005479045) | IFNSTNRLFQYASTDMDKVLLK |
| | | | | YTEYSEPHESRTNTDILETLRRK |
| | | | | GLGLDGSELDSEESMQVATDKY |
| | | | | PLSEGMDLSVARQRFYGPSLLSP |
| | | | | EAQFLVSAGCENGFPNSSGSGM |
| | | | | ASHRPPSFKSLNSRSSSASPAAPH |
| | | | | AHTAFMSPHSGIGYSVFSHGNLN |
| | | | | RALDMKSPPPLNVGTENLRADG |
| | | | | ANQAMGATRANHNSARGLLYQ |
| | | | | GLHSSSSMVAMGKAGLLSHSLG |
| | | | | GYGLPSPGASEYSQPGFYHSVSL |
| | | | | QRGTVNPWQAAQPPQEPHGPHI |
| | | | | NPVESSGGCSFPSQSCTPTSPHLP |
| | | | | SLNLSIKSERSSPEHMASPTSPPL |
| | | | | HHLRQHSPMSNPDSARHTPPDT |
| | | | | HPANGTKEFAKTSYPQDQEEGG |
| | | | | QSLRQLEMSDGWQR (SEQ ID |
| | | | | NO: 18) |
| PPARG | Gallus | XP_046781908.1 | XM_046925952 | MVDTEMPFWPVNFGISPVDLSA |
| | (chicken) | (SEQ ID NO: 19) | (encodes | MDDHMHSFDIKPFTTVDFSSISSP |
| | | | XP_046781908.1) | HYEDIPLGRADQTSIDYKYDIKL |
| | | | | QDCQSAIKMEPPSPPYFSEKVQL |
| | | | | YNKPHEESSNSLMAIECRVCGD |
| | | | | KASGFHYGVHACEGCKGFFRRT |
| | | | | IRLKLIYDRCDLNCRIHKKSRNK |
| | | | | CQYCRFQKCLAVGMSHNAIRFG |
| | | | | RMPQAEKEKLLAEISSDIDQLNP |
| | | | | ESADLRALAKHLYDSYIKSFPLT |
| | | | | KAKARAILTGKTTDKSPFVIYDM |
| | | | | NSLRMGEDQIKCKHASPLQEQN |
| | | | | KEVAIRIFQRCQFRSVEAVQEITE |
| | | | | FAKNIPGFVNLDLNDQVTLLKY |
| | | | | GVHEIIYTLLASLMNKDGVLISD |
| | | | | GQGFMTREFLKSLRKPFCDFME |
| | | | | PKFEFA VKFNALELDDSDLAIFIA |
| | | | | VIILSGDRPGLLNVKPIEDIQDNL |
| | | | | LQALELQLKLNHPESSQLFAKLL |
| | | | | QKMTDLRQIVTEHVQLLQIIKKT |
| | | | | ETDMSLHPLLQEIYKDLY (SEQ |
| | | | | ID NO: 19) |
| PPARG | Anas | XP_027323025.1 | XM_027467224 | MVDTEMPFWPINFGISPVDLSA |
| | platyrhynchos | (SEQ ID NO: 20) | (encodes | MDDHTHSFDIKPFTTVDFSSISSP |
| | (Duck) | | XP_027323025.1) | HYEDIPLARTDQTSIDYKYDIKL |
| | | | | QDCQSAIKMEPPSPPYFSEKVQL |
| | | | | YNKPHEESSNSLMAIECRVCGD |
| | | | | KASGFHYGVHACEGCKGFFRRT |
| | | | | IRLKLIYDRCDLNCRIHKKSRNK |
| | | | | CQYCRFQKCLAVGMSHNAIRFG |
| | | | | RMPQAEKEKLLAEISSDIDQLNP |
| | | | | ESADLRALAKHLYDSYIKSFPLT |
| | | | | KAKARAILTGKTTDKSPFVIYDM |
| | | | | NSLMMGEDQIKCKHVSPLQEQN |
| | | | | KEVAIRIFQRCQFRSVEAVQEITE |
| | | | | FAKNIPGFVNLDLNDQVTLLKY |
| | | | | GVHEIIYTLLASLMNKDGVLISD |
| | | | | GQGFMTREFLKSLRKPFCDFME |
| | | | | PKFEFAVKFNALELDDSDLAIFIA |
| | | | | VIILSGDRPGLLNVKPIEDIQDNL |
| | | | | LQALELQLKLNHPESSQLFAKLL |
| | | | | QKMTDLRQIVTEHVQLLQIIKKT |
| | | | | ETDMSLHPLLQEIYKDLY (SEQ |
| | | | | ID NO: 20) |
| PPARG | Meleagris | XP_003210088.2 | XM_003210040 | MSGHYHLGTSAQLIGEDGHCCT |
| | gallopavo | (SEQ ID NO: 21) | (encodes | YLSPCQNKGSSQSNRAEITMVDT |
| | (Turkey) | | XP_003210088.2) | EMPFWPVNFGISPVDLSAMDDH |
| | | | | MHSFDIKPFTTVDFSSISSPHYED |
| | | | | IPLGRADQTSIDYKYDIKLQDCQ |
| | | | | SAIKMEPPSPPYFSEKVQLYNKP |
| | | | | HEESSNSLMAIECRVCGDKASGF |
| | | | | HYGVHACEGCKGFFRRTIRLKLI |
| | | | | YDRCDLNCRIHKKSRNKCQYCR |
| | | | | FQKCLAVGMSHNAIRFGRMPQA |
| | | | | EKEKLLAEISSDIDQLNPESADLR |
| | | | | ALAKHLYDSYIKSFPLTKAKAR |
| | | | | AILTGKTTDKSPFVIYDMNSLRM |
| | | | | GEDQIKCKHASPLQEQNKEVAIR |
| | | | | IFQRCQFRSVEAVQEITEFAKNIP |
| | | | | GFVNLDLNDQVTLLKYGVHEIIY |
| | | | | TLLASLMNKDGVLISDGQGFMT |
| | | | | REFLKSLRKPFCDFMEPKFEFAV |
| | | | | KFNALELDDSDLAIFIAVIILSGD |
| | | | | RPGLLNVKPIEDIQDNLLQALEL |
| | | | | QLKLNHPESSQLFAKLLQKMTD |
| | | | | LRQIVTEHVOLLQIIKKTETDMS |
| | | | | LHPLLQEIYKDLY |
| | | | | (SEQ ID NO: 21) |
| PPARG | Sus scrofa | NP_999544.1 | NM_214379 | MGETLGDSLIDPESDAFDTLSAN |
| | (Porcine) | (SEQ ID NO: 22) | (encodes | ISQEVTMVDTEMPFWPTNFGISS |
| | | | NP_999544.1) | VDLSVMDDHSHSFDIKPFTTVDF |
| | | | | SSISTPHYEDIPFPRADPMVADY |
| | | | | KYDLKLQDYQSAIKVEPVSPPY |
| | | | | YSEKTQLYNKPHEEPSNSLMAIE |
| | | | | CRVCGDKASGFHYGVHACEGC |
| | | | | KGFFRRTIRLKLIYDRCDLNCRIH |
| | | | | KKSRNKCQYCRFQKCLAVGMS |
| | | | | HNAIRFGRMPQAEKEKLLAEISS |
| | | | | DIDQLNPESADLRALAKHLYDS |
| | | | | YIKSFPLTKAKARAILTGKTTDK |
| | | | | SPFVIYDMNSLMMGEDKIKFKHI |
| | | | | TPLQEQSKEVAIRIFQGCQFRSVE |
| | | | | AVQEITEYAKNIPGFVNLDLNDQ |
| | | | | VTLLKYGVHEIIYTMLASLMNK |
| | | | | DGVLISEGQGFMTREFLKSLRKP |
| | | | | FGDFMEPKFEFAVKFNALELDD |
| | | | | SDLAIFIAVIILSGDRPGLLNVKPI |
| | | | | EDIQDNLLQALELQLKLNHPESS |
| | | | | QLFAKLLQKMTDLRQIVTEHVQ |
| | | | | LLQVIKKTETDMSLHPLLQEIYK |
| | | | | DLY |
| | | | | (SEQ ID NO: 22) |
| PPARG | Bos taurus | NM_181024 | NM_181024 | MGETLGDALIDPESEPFAVTVSA |
| | (Bovine) | (SEQ ID NO: 23) | (encodes | RTSQEITMVDTEMPFWPTNFGIS |
| | | | NP_851367.1) | SVDLSMMDDHSHAFDIKPFTTV |
| | | | | DFSSISTPHYEDIPFPRADPMVAD |
| | | | | YKYDLKLQEYQSAIKVEPVSPPY |
| | | | | YSEKTQLYSKPHEEPSNSLMAIE |
| | | | | CRVCGDKASGFHYGVHACEGC |
| | | | | KGFFRRTIRLKLIYDRCDLNCRIH |
| | | | | KKSRNKCQYCRFQKCLAVGMS |
| | | | | HNAIRFGRMPQAEKEKLLAEISS |
| | | | | DIDQLNPESADLRALAKHLYDS |
| | | | | YIKSFPLTKAKARAILTGKTTDK |
| | | | | SPFVIYDMNSLMMGEDKIKFKHI |
| | | | | SPLQEPSKEVAIRIFQGCQFRSVE |
| | | | | AVQEITEYAKNIPGFVNLDLNDQ |
| | | | | VTLLKYGVHEIIYTMLASLMNK |
| | | | | DGVLISEGQGFMTREFLKSLRKP |
| | | | | FGDFMEPKFEFAVKFNALELDD |
| | | | | SDLAIFIAVIILSGDRPGLLNVKPI |
| | | | | EDIQDNLLQALELQLKLNHPESS |
| | | | | QLFAKLLQKMTDLRQIVTEHVQ |
| | | | | LLQVIKKTETDMSLHPLLQEIYK |
| | | | | DLY (SEQ ID NO: 23) |
| PPARG | Oreochromis | XP_025762757.1 | XM_025906972 | MQTPGRDFTHQTDHSFSDMVDT |
| | niloticus | (SEQ ID NO: 24) | (encodes | QQLLAWPVGFSLNTADLPELED |
| | (Tilapia) | | XP_025762757.1) | SSHSLEMKHLSTFDYASISSSSIPS |
| | | | | SLSPPLVSSIPSAGVAYDLSPPQS |
| | | | | EEHLTNMDYTNMHSYRTQPDM |
| | | | | FNSIKLEPESPPQYSDSPVLSKLP |
| | | | | DDTSAAALNIECRVCGDKASGF |
| | | | | HYGVHACEGCKGFFRRTIRLKL |
| | | | | VYDHCDLHCRIHKKSRNKCQYC |
| | | | | RFQKCLNVGMSHNAIRFGRMPQ |
| | | | | AEKEKLLAEFSSDMEHMHPEAA |
| | | | | DLRALARHLYEAYLKYFPLTKA |
| | | | | KARAILSGKTGDNVPFVIHDMK |
| | | | | SLMEGEQFINCKQIPVPEHHQQQ |
| | | | | QHQTSTLPPVHGGYTETHPMSES |
| | | | | EVMRMTGFGGHGEADAVELRF |
| | | | | FQSCQSRSAEAVREVTEFAKSIP |
| | | | | GFVDLDLNDQVTLLKYGVIEVLI |
| | | | | IMMSPLMNKDGTLISYGQIFMTR |
| | | | | EFLKSLRKPFCQMMEPKFEFSVK |
| | | | | FNTLELDDSDMALFLAVIILSGD |
| | | | | RPGLLNVKPIEQLQETVLHSLEL |
| | | | | QLKLNHPDSLQLFAKLLQKMTD |
| | | | | LRQIVTDHVHLIQLLKKTEIDMC |
| | | | | LHPLLQEIMKDLY (SEQ ID NO: |
| | | | | 24) |

| HSPA1A Promoter | ATCCAGTTTGATACGGTTCGGATGGGGAGCCC |
|---|---|
| SEQ ID NO: 29 | CTATAACTTGGGGCTCCTTCAATAGCCAAACT |
| | GCGCAGGCGGTGTCCCCCCACCCCCCACCCGC |
| | CCCGCCCTGCGACTTTGAGCTGGGTCCAGACC |
| | TAGCATCCTAATTCTCTACTAGCCCGTGAGGTC |
| | AGAGGCAGCACCTCCATTGTAACGCGACTAGA |
| | GCAGGGCGGCGTCAACACCACCGCCCGAAGTC |
| | CCGACCCACCAGCCCCTCCTACCGCTTCCCCTC |
| | CCATTACCCCTTTCCGAGACAGTGCCAGCTAG |
| | CAAGTCCCAGAAGAGTCTGGCGAGTTCTGGGA |
| | GGAGTGGCATCCAGGGCGCCGATTGGTCCCAG |
| | AAAGCCAGGGGGCAGGACTTGAGGCGAAACC |
| | CCTGGAATATTCCCGACCTGGCAGCCCCACTG |
| | AACTCGGTCATTGGCTGACGAGGGAAAAGGCG |
| | GGGCTTGATGAAGAATTATAAACACAGAGCCG |
| | CCTGAGGAGAAACAGCAGCCTGGAGAGAGCT |
| | GATAAAACTTGCGGCTTAGTCCGTGAGAACAG |
| | CTTCCGCAGACCCGCTATCTCCAAGGACCGCC |
| | CCGAGGGGCACCAGAGCTTCACGTCGTTGATC |
| | CTGTGGGCCGTTTTCAGGTTTGAAGCTTATCTC |
| | GGAGCCGAAAAGGCAGGGCACCGGC |

### 5.1.2. Culture, Transfection, and Selection

Provided herein are methods including an animal (non-human) cell cultivation method comprising: (a) cultivating avian, bovine, non-human mammalian, or non-human animal cells comprising a nucleic acid encoding a factor or biomarker associated with a fat or muscle phenotype operably linked to a heat shock promoter in a bioreactor for a first time period at a proliferation temperature; (b) exposing the cells to a heat shock temperature for a second time period, thereby inducing expression of the factor or biomarker; (c) separating at least a portion of the heat shocked cells from the media; and (d) harvesting the separated, heat shocked cells for formulation into a comestible food product. Such methods are advantageous for the product of food products in addressing challenges for the mass production of cells, including yield, scale, and the efficient production of cells with fat or muscle phenotypes using methods that are compatible with food production, for example, don't involve induction agents, such as non-GRAS small molecules that may be undesirable in food production. In embodiments, step b is carried out in a differentiation system for the second time period, wherein the differentiation system comprises culturing the cells in a tank or in a pipe through which the cells flow. In other embodiments, after step b, the cells are cultivated for a finishing time period, at a proliferation temperature between 1 and 14 days. In embodiments, prior to step d, the cells exhibit a 0.5 fold to 30 fold increase in expression of factors or biomarkers associated with a fat or muscle phenotype compared to non-heat shocked cells. In some embodiments, prior to step d, the cells exhibit phenotypic and genotypic characteristics of adipocytes and/or myotubes.

In embodiments, the proliferation temperature is at or between 35° C.-40° C. and may be determined to optimize cell growth while not inducing heat shock promoter regulated expression. In embodiments, the cells are cultured for first time or first time period at proliferation temperatures of 1 to 14 days. In embodiments, the first time period is for 1 to 14 days, 1 to 13 days, 1 to 12 days, 1 to 11 days, 1 to 10 days, 1 to 9 days, 1 to 8 days, 1 to 7 days, 1 to 6 days, 1 to 5 days, 1 to 4 days, 1 to 3 days, 1 to 2 days, 2 to 14 days, 2 to 13 days, 2 to 12 days, 2 to 11 days, 2 to 10 days, 2 to 9 days, 2 to 8 days, 2 to 7 days, 2 to 6 days, 2 to 5 days, 2 to 4 days, 2 to 3 days, 3 to 14 days, 3 to 13 days, 3 to 12 days, 3 to 11 days, 3 to 10 days, 3 to 9 days, 3 to 8 days, 3 to 7 days, 3 to 6 days, 3 to 5 days, 3 to 4 days, 4 to 14 days, 4 to 13 days, 4 to 12 days, 4 to 11 days, 4 to 10 days, 4 to 9 days, 4 to 8 days, 4 to 7 days, 4 to 6 days, 4 to 5 days, 5 to 14 days, 5 to 13 days, 5 to 12 days, 5 to 11 days, 5 to 10 days, 5 to 9 days, 5 to 8 days, 5 to 7 days, 5 to 6 days, 6 to 14 days, 6 to 13 days, 6 to 12 days, 6 to 11 days, 6 to 10 days, 6 to 9 days, 6 to 8 days, 6 to 7 days, 7 to 14 days, 7 to 13 days, 7 to 12 days, 7 to 11 days, 7 to 10 days, 7 to 9 days, 7 to 8 days, 8 to 14 days, 8 to 13 days, 8 to 12 days, 8 to 11 days, 8 to 10 days, 8 to 9 days, 9 to 14 days, 9 to 13 days, 9 to 12 days, 9 to 11 days, 9 to 10 days, 10 to 14 days, 10 to 13 days, 10 to 12 days, 10 to 11 days, 11 to 14 days, 11 to 13 days, 11 to 12 days, 12 to 14 days, 12 to 13 days, or 13 to 14 days.

In other embodiments, the second time period is the time it takes to activate the heat shock promoter. In embodiments, the second time period is 1 to 6 hours. In some embodiments, the second time period is 1 to 6 hours, 1 to 5 hours, 1 to 4 hours, 1 to 3 hours, 1 to 2 hours, 2 to 6 hours, 2 to 5 hours, 2 to 4 hours, 2 to 3 hours, 3 to 6 hours, 3 to 5 hours, 3 to 4 hours, 4 to 6 hours, 4 to 5 hours, or 5 to 6 hours. In other embodiments, the cells are cultured at a heat shock temperature for a second time period for 1 to 24 hours. In embodiments, the cells are cultured at a heat shock temperature for about 1 hour, for about 1.5 hours, for about 2 hours, for about 2.5 hours, for about 3 hours, for about 3.5 hours, for about 4 hours, for about 4.5 hours, for about 5 hours, for about 5.5 hours, for about 6 hours, for about 6.5 hours, for about 7 hours, for about 7.5 hours, for about 8 hours, for about 8.5 hours, for about 9 hours, for about 9.5 hours, for about 10 hours, for about 10.5 hours, for about 11 hours, for about 11.5 hours, for about 12 hours, for about 12.5 hours, for about 13 hours, for about 13.5 hours, for about 14 hours, for about 14.5 hours, for about 15 hours, for about 15.5 hours, for about 16 hours, for about 16.5 hours, for about 17 hours, for about 17.5 hours, for about 18 hours, for about 18.5 hours, for about 19 hours, for about 19.5 hours, for about 20 hours, for about 20.5 hours, for about 21 hours, for about 21.5 hours, for about 22 hours, for about 22.5 hours, for about 23 hours, for about 23.5 hours, for about 24 hours.

In embodiments, the proliferation temperature is between or at 35° C. and 40° C. In further embodiments, the proliferation temperature is between 35° C. and 40° C., between 35° C. and 39° C., between 35° C. and 38° C., between 35° C. and 37° C., or between 35° C. and 36° C. In even further embodiments, the proliferation temperature is 35° C., 36° C., 37° C., 38° C., 39° C., or 40° C. In embodiments, the heat shock temperature is between or at 37° C. and 45° C. In further embodiments, the heat shock temperature is between 37° C. and 45° C., between 37° C. and 44° C., between 37° C. and 43° C., between 37° C. and 42° C., between 37° C. and 41° C., between 37° C. and 40° C., between 37° C. and 39° C., or between 37° C. and 38° C. In even further embodiments, the heat shock temperature is 37° C., 38° C., 39° C., 40° C., 41° C., 42° C., 43° C., 44° C., and 45° C. In embodiments, the proliferation temperature range and heat shock temperature range overlap, and the factor or biomarker associated with a fat or muscle phenotype is expressed at a heat shock temperature 3° C. to 7° C. higher than the proliferation temperature. In embodiments, the heat shock temperature is at least 3° C. higher than the proliferation temperature. In further embodiments, the heat shock temperature is at or between 37° C. and 43° C. and wherein the heat shock temperature is at least 3° C. higher than the proliferation. In other embodiments, a substantial portion of the cellular population exhibits expression of the gene operably linked to the heat shock promoter at 43° C. in even further embodiments, the substantial portion is at least 51% of the cellular popular.

In some embodiments, the expression of one or more biomarkers associated with a fat or muscle phenotype is at 0.5 to 30 fold higher in heat shocked cells than non-transfected cells or cells cultured at a standard temperature for a time. In other embodiments, the expression of the expression of one or more biomarkers associated with a fat or muscle phenotype is 0.5 fold, 1 fold, 2 fold, 3 fold, 4 fold, 5 fold, 6 fold, 7 fold, 8 fold, 9 fold, 10 fold, 11 fold, 12 fold, 13 fold, 14 fold, 15 fold, 16 fold, 17 fold, 18 fold, 19 fold, 20 fold, 21 fold, 22 fold, 23 fold, 24 fold, 25 fold, 26 fold, 27 fold, 28 fold, 29 fold or 30 fold higher in heat shocked cells than non-transfected cells or cells cultured at a standard temperature for a time.

In embodiments, the cells have a basal doubling less than 24 hours before exposure to the heat shock temperature, wherein the cells return to the basal doubling time within 6 days of exposure to the heat shock temperature.

In embodiments, the heat shock promoter is homologous to the species of the cells. In other embodiments, the heat shock promoter is heterologous to the species of the cells.

In embodiments, the proliferation temperature is at or between 35° C. and 40° C. to a target density. In further embodiments, the target cell density is about 0.1 mil/mL cells to about 70 mil/mL.

Provided herein are methods of selecting cell lines with an inducible heat shock system, comprising: (a) transfecting cells, comprising avian, bovine, non-human mammalian, or non-human animal cells, with a vector construct encoding a fluorescent protein and a heat shock promoter operably linked to a nucleic acid encoding a protein of interest, wherein the fluorescent protein is substantially unintegrated; (b) selecting cells expressing the fluorescent protein after a first period; and (c) discarding cells expressing the fluorescent protein after a second period, wherein a substantial majority of remaining cells express the heat shock promoter and the protein of interest and not the fluorescent protein.

Provided herein are methods of selecting cell lines with heat shock inducible nucleic acids encoding one or more factors with a fat or muscle phenotype, which cell lines do not exhibit significant leaky expression (i.e., insignificant or minimal expression that is no more than 0.1%, 1%, 5% or 10% of the expression at heat shock temperatures or is indetectable) at non-induction temperatures, comprising: (a) transfecting avian, bovine, non-human mammalian, or non-human animal cells with a vector construct comprising a nucleic acid encoding one or more factors or biomarkers associated with a fat or muscle phenotype operably linked to a heat shock promoter; (b) selecting cells that comprise the nucleic acid and express the one or more factors or biomarkers associated with a fat or muscle phenotype when exposed to temperatures that activate the heat shock promoter; and (c) clonally selecting cells that do not exhibit significant leaky expression of the one or more factors or biomarkers associated with a fat or muscle phenotype when cultured at a temperature that does not activate the heat shock promoter. In embodiments, the cells from the selected cell line do not exhibit increased doubling time (i.e., poorer performance) when cultured at non-heat shock temperatures, including when the selected cells have doubling times at non-heat shock culture temperatures that are no more than 0.1%, 1%, 5%, or 10% greater than comparable cells which do not comprise the nucleic acid encoding the factor operably linked to a heat shock promoter.

Also provided herein is a method of producing a cell line with heat shock inducible nucleic acids encoding one or more factors or biomarkers associated with a fat or muscle phenotype comprising: (a) transfecting avian, bovine, non-human mammalian, or non-human animal cells with a vector construct encoding one or more factors or biomarkers associated with a fat or muscle phenotype operably linked to a heat shock promoter, (b) selecting clonally derived functional cells that comprise the nucleic acid encoding the one or more factors or biomarkers associated with a fat or muscle phenotype operably linked to the heat shock promoter and only express the one or more factors or biomarkers associated with a fat or muscle phenotype to a significant degree at heat shock temperatures, wherein the cells express less than 0.1%, 1%, or 10% of the one or more factors or biomarkers associated with a fat or muscle phenotype when cultured at non-inducing temperatures than when the cells are cultured at temperatures that activate the heat shock promoter and maintain cell viability.

In embodiments, methods of selecting cell lines with heat shock inducible nucleic acids encoding PPARG, CEBPA, PAX7, MYOD, MYH2, and/or MEF2B, which do not exhibit significant leaky expression, comprise: (a) transfecting avian cells with a vector construct comprising nucleic acids encoding PPARG, CEBPA, PAX7, MYOD, MYH2, and/or MEF2B operably linked to the heat shock promoter; (b) selecting cells comprising the nucleic acid encoding PPARG, CEBPA, PAX7, MYOD, MYH2, and/or MEF2B but not expressing PPARG, PAX7, MYOD, MYH2, and/or MEF2B at proliferation temperatures or temperatures that do not substantially activate the heat shock promoter; and (c) culturing the cells at a heat shock temperature, including at temperature of 43° C. for about 3 hours, to activate the HSP1A1 promoter, and (d) selecting cells that express desired levels of PPARG, CEBPA, PAX7, MYOD, MYH2, and/or MEF2B at heat shock temperatures. In embodiments, the PPARG, CEBPA, PAX7, MYOD, MYH2, and/or MEF2B and promoter are, or are derived from, chicken genes and promoters to chicken cells. In embodiments, the cells are fibroblasts. In embodiments, the heat shock promoter is a HSP1A1 promoter. In additional embodiments, the heat shock promoter comprises the nucleotide sequence of SEQ ID NO: 29.

In embodiments, methods of selecting cell lines with heat shock inducible expression of PPARG, CEBPA, PAX7, MYOD, MYH2, and/or MEF2B which do not exhibit significant leaky expression, comprise: (a) transfecting bovine cells with a plasmid vector construct comprising nucleic acids encoding PPARG, CEBPA, PAX7, MYOD, MYH2, and/or MEF2B operably linked to the heat shock promoter; (b) selecting cells comprising the nucleic acid encoding the PPARG, CEBPA, PAX7, MYOD, MYH2, and/or MEF2B but not substantially expressing the PPARG, CEBPA, PAX7, MYOD, and/or MEF2B at proliferation temperatures or temperatures that do not activate the heat shock promoter; and (c) culturing the cells at a heat shock temperature of about 43° C. average for about 3 hours to activate the HSP1A1 promoter and (d) selecting cells that express desired levels of PPARG, CEBPA, PAX7, MYOD, MYH2, and/or MEF2B at heat shock temperatures. In embodiments, the PPARG, CEBPA, PAX7, MYOD, MYH2, and/or MEF2B and promoter are, or are derived from, bovine coding sequences and promoters. In embodiments, the cells are fibroblasts. In other embodiments, the heat shock promoter is a HSP1A1 promoter. In additional embodiments, the heat shock promoter comprises the nucleotide sequence of SEQ ID NO: 29.

In embodiments, a method of producing a cell line with heat shock inducible expression of PPARG, CEBPA, PAX7, MYOD, MYH2, and/or MEF2B, which do not exhibit significant leaky expression, comprises: (a) transfecting avian, bovine, non-human mammalian, or non-human animal cells with a vector construct comprising nucleic acids encoding PPARG, CEBPA, PAX7, MYOD, MYH2, and or MEF2B operably linked to the heat shock promoter; (b) selecting cells comprising the nucleic acid encoding the factors but not substantially expressing the factors at proliferation temperatures or temperatures that do not activate the heat shock promoter; and (c) selecting clonally derived functional cells that express PPARG, CEBPA, PAX7, MYOD, MYH2, or MEF2B at a detectable level at 43° C. or a temperature that activates the heat shock promoter, wherein the cells express less than 0.1%, 1%, 5%, or 10% of the expression of PPARG, CEBPA, PAX7, MYOD, MYH2, and/or MEF2B when cultured at non-inducing temperatures than when the cells are cultured at temperatures that activate the heat shock promoter and maintain cell viability. In embodiments, the PPARG, CEBPA, PAX7, MYOD, MYH2, and/or MEF2B and promoter are, or are derived from, genes and promoters from chicken. In other embodiments, the heat shock promoter is a HSP1A1 promoter. In additional embodiments, the heat shock promoter comprises the nucleotide sequence of SEQ ID NO: 29.

In embodiments, a method of producing a cell line with heat shock inducible expression of PPARG, CEBPA, PAX7, MYOD, MYH2, and/or MEF2B comprises: (a) transfecting bovine cells with a plasmid vector construct comprising nucleic acids encoding PPARG, CEBPA, PAX7, MYOD, MYH2, and/or MEF2B operably linked to an HSP1A1 promoter; (b) selecting cells comprising the nucleic acid encoding the factors but not significantly expressing the factors at proliferation temperatures or temperatures that do not activate the heat shock promoter; and (c) selecting clonally derived functional cells that express PPARG, CEBPA, PAX7, MYOD, MYH2, or MEF2B at a significant levels at 43° C. or at a temperature that activates the heat shock promoter, wherein the cells express less than 0.1%, 1%, 5%, or 10% of the factors when cultured at non-inducing temperatures than when the cells are cultured at temperatures that activate the heat shock promoter and maintain cell viability. In embodiments, the PPARG, CEBPA, PAX7, MYOD, MYH2, and/or MEF2B and promoter are, or are derived from, bovine genes and promoters. In embodiments, the cells are fibroblasts. In additional embodiments, the heat shock promoter comprises the nucleotide sequence of SEQ ID NO: 29.

### 5.1.2.1. Culturing Cells

The methods provided herein include culturing avian, bovine, non-human mammalian, or non-human animal cells, such that the cells proliferate to form a biomass and then inducing muscle and or fat phenotype expression in the cells which are used for cultured cell based food products.

Also provided herein are methods that include culturing the cells comprising a nucleic acid encoding one, two, three, or four factors or biomarkers associated with a fat or muscle phenotype operably linked to a heat shock promoter at a temperature that activates the heat shock promoter and maintains cell viability for a time period, wherein the activated heat shock promoter induces expression of the one or more factors or biomarkers associated with a fat or muscle phenotype at a level that promotes expression of a fat or muscle phenotype by the cultured cells.

In some embodiments, the non-human mammalian or non-human animal cells are derived from any non-human animal, including but not limited to fish, shellfish, cattle, pigs, sheep, goats, deer, rabbits, and poultry. In some embodiments, the non-human mammal cells are derived from any non-human mammal, including but not limited to cattle, pigs, sheep, goats, deer, rabbits, and poultry. In some embodiments, the avian cells are chicken cells.

In some other embodiments, the avian, bovine, non-human mammalian, or non-human animal cells are fibroblasts or may be a stem cell, including pluripotent stem cells. In other embodiments, the avian, bovine, or non-human are fibroblasts. In embodiments, the fibroblasts can express phenotypes associated with fat or muscle cells, e.g., differentiate into fat or muscle cell types.

In some embodiments, the cells are cultured in serum free media. Serum free media refers to media that is substantially or virtually free of serum. In some embodiments, the cell culture media is hormone free. In some embodiments, the cell culture media is heavy metal free. In some embodiments, the culture media contains insufficient heavy metal concentrations to induce substantial expression at the first temperature, i.e. the proliferation temperature. In some embodiments, the cell culture media is antibiotic free. In other embodiments, the cell culture media is animal-component free (ACF).

The cells can be grown in any vessel suitable for suspension culture. Non-limiting examples of suitable vessels include tubes, flasks, multi-well plates, dishes, vats, bioreactors, and the like. In embodiments, the bioreactor has a capacity between about 1,000 and 100,000 liters.

In embodiments, the cells are grown in serum free media to a cell density of at least 0.1 mil/mL, 0.2 mil/mL, 0.3 mil/mL, 0.4 mil/mL, 0.5 mil/mL, 1 mil/mL, 2 mil/mL, 3 mil/mL, 4 mil/mL, 5 mil/mL, 6 mil/mL, 7 mil/mL, 8 mil/mL, 9 mil/mL, 10 mil/mL, 11 mil/mL, 12 mil/mL, 13 mil/mL, 14 mil/mL, 15 mil/mL, 16 mil/mL, 17 mil/mL, 18 mil/mL, 19 mil/mL, 20 mil/mL, 21 mil/mL, 22 mil/mL, 23 mil/mL, 24 mil/mL, 25 mil/mL, 26 mil/mL, 27 mil/mL, 28 mil/mL, 29 mil/mL, 30 mil/mL, 31 mil/mL, 32 mil/mL, 33 mil/mL, 34 mil/mL, 35 mil/mL, 36 mil/mL, 37 mil/mL, 38 mil/mL, 39 mil/mL, 40 mil/mL, 41 mil/mL, 42 mil/mL, 43 mil/mL, 44 mil/mL, 45 mil/mL, 46 mil/mL, 47 mil/mL, 48 mil/mL, 49 mil/mL, 50 mil/mL, 51 mil/mL, 52 mil/mL, 53 mil/mL, 54 mil/mL, 55 mil/mL, 56 mil/mL, 57 mil/mL, 58 mil/mL, 59 mil/mL, 60 mil/mL, 61 mil/mL, 62 mil/mL, 63 mil/mL, 64 mil/mL, 65 mil/mL, 66 mil/mL, 67 mil/mL, 68 mil/mL, 69 mil/mL, or at least about 70 mil/mL.

In some embodiments, the cells are in suspension, adherent, in suspension aggregates, or on suspension microcarriers. In embodiments, the cells are adapted for suspension culture and are anchorage independent. In embodiments, the cells are adherent, anchorage dependent, and adhere to plastic, glass, or other surfaces that support cell culture. In embodiments, the cells are suspension aggregates, wherein the cells are clustered and adhered together to form aggregates and adapted for suspension culture. In embodiments, the suspension aggregates are anchorage independent. In embodiments, the cells are cultured on suspension microcarriers. Microcarriers are used to expand anchorage dependent cells in large-scale suspension bioreactors.

In embodiments, the bovine cells are cultured at a proliferation temperature of 37° C. (also referred to interchangeably as a growing or cultivation temperature) for standard cultivation for proliferation under conditions that do not activate (or do not significantly activate) heat shock promoter induction. In embodiments, the bovine cells are cultured at 43° C. to activate the transfected heat shock promoter, which induces expression of the one or more factors or biomarkers associated with a fat or muscle phenotype. In embodiments, the avian cells are cultured at a proliferation temperature at or between 37C to 39° C. for standard cultivation for proliferation under conditions that do not activate (or do not significantly activate) heat shock promoter induction. In embodiments, the avian cells are cultured at 43° C. to activate the transfected heat shock promoter, which induces expression of the one or more factors or biomarkers associated with a fat or muscle phenotype. In embodiments, the proliferation and heat shock inducible temperature are different in cultivation infrastructure for cells derived from avian, bovine, or non-human species. In embodiments, the proliferation and heat shock inducible temperature are different in cultivation infrastructure for cells of different tissue types (e.g. adipocytes and myotubes). In embodiments, the proliferation and heat shock inducible temperature are different in cultivation infrastructure for cells of different species (e.g. avian and bovine). The heat shock temperature should be a temperature that results in gene expression from the heat shock promoter but does not significantly impact cell viability. In embodiments, the proliferation temperature is between or at 35° C. and 40° C. In further embodiments, the proliferation temperature is between 35° C. and 40° C., between 35° C. and 39° C., between 35° C. and 38° C., between 35° C. and 37° C., or between 35° C. and 36° C. In even further embodiments, the proliferation temperature is 35° C., 36° C., 37° C., 38° C., 39° C., or 40° C. In embodiments, the heat shock temperature is between or at 37° C. and 45° C. In further embodiments, the heat shock temperature is between 37° C. and 45° C., between 37° C. and 44° C., between 37° C. and 43° C., between 37° C. and 42° C., between 37° C. and 41° C., between 37° C. and 40° C., between 37° C. and 39° C., or between 37° C. and 38° C. In even further embodiments, the heat shock temperature is 37° C., 38° C., 39° C., 40° C., 41° C., 42° C., 43° C., 44° C., and 45° C. In embodiments, the proliferation temperature range and heat shock temperature range overlap, and the factor or biomarker associated with a fat or muscle phenotype is expressed at a heat shock temperature 3° C. to 7° C. higher than the proliferation temperature. In embodiments, the heat shock temperature is at least 3° C. higher than the proliferation temperature. In embodiments, a substantial portion of the cellular population exhibits expression of the gene operably linked to the heat shock promoter at 43° C. in other embodiments, a substantial portion is 51% of the cellular population.

In some embodiments, the adherent non-human mammalian or non-human animal cells are transfected with a vector construct comprising: (a) a heat shock promoter and (b) a nucleic acid encoding one or more factors or biomarkers associated with a fat or muscle phenotype operably linked to the heat shock promoter. In further embodiments, the expression of the nucleic acid is induced by a heat shock. In even further embodiments, the expression of the nucleic acid is higher in heat shock cells compared to non-heat shock cells. In embodiments, the heat shock promoter is a HSP1A1 promoter or an HSF-1 promoter. In additional embodiments, the heat shock promoter comprises the nucleotide sequence of SEQ ID NO: 29.

In some embodiments, the non-human mammalian or non-human animal cells cultivated on microcarriers are transfected with a vector construct comprising: (a) a heat shock promoter and (b) a nucleic acid encoding one or more factors or biomarkers associated with a fat or muscle phenotype operably linked to the heat shock promoter. In further embodiments, the expression of the nucleic acid is induced by a heat shock. In even further embodiments, the expression of the nucleic acid is higher in heat shock cells compared to non-heat shock cells. In embodiments, the heat shock promoter is a HSP1A1 promoter or an HSF-1 promoter. In additional embodiments, the heat shock promoter comprises the nucleotide sequence of SEQ ID NO: 29.

In some embodiments, the non-human mammalian or non-human animal cells in suspension are transfected with a vector construct comprising: (a) a heat shock promoter and (b) a nucleic acid encoding one or more factors or biomarkers associated with a fat or muscle phenotype operably linked to the heat shock promoter. In further embodiments, the expression of the nucleic acid is induced by a heat shock. In embodiments, the heat shock promoter is a HSP1A1 promoter or an HSF-1 promoter. In even further embodiments, the expression of the nucleic acid is higher in heat shock cells compared to non-heat shock cells.

### 5.1.2.2. Transfection

The methods provided herein include transfecting cells with a nucleic acid encoding one or more factors or biomarkers associated with a fat or muscle phenotype (with integration targeted to be adjacent to a homologous or heterologous heat shock promoter) or one or more factors or biomarkers associated with a fat or muscle phenotype, wherein the nucleic acid is operably linked to a heat shock promoter, e.g. a homologous heat shock promoter (even if from the same species as the cell but in a different episomal or genomic context) or a heat responsive functional portion thereof.

Transfection is the process of introducing nucleic acids into a cell. Transfection can be performed using any known method in the art. Non-limiting examples include, but not limited to, viral based transduction, such as retroviral and lentiviral transduction for stable transduction, and adenovirus, adeno-associated virus (AAV), or helper virus for transient transduction. Non-viral methods include but are not limited to calcium phosphate, microparticles, nanoparticles, cationic polymer transfection, dendrimers, cationic lipid transfection, electroporation, laser, cellular injection, sonoporation, optical transfection, lipid nanoparticles, hydrodynamic delivery, magnetofection, or self-delivering FANA ASOs. In embodiments wherein the fibroblast cells are transfected with lipid transfection, the ratio of DNA:reagent can be about 1:5, 1:4, 1:3, 1:3.5, 1:2.5, 1:2, 1:1.5, 1:1, or about 1:0.5.

In some embodiments, the nucleic acid is stably integrated into the genome of the selected cell lines.

In some embodiments, the clonal cell lines exhibit at least one or more vector copy numbers.

The methods provided herein include introducing into, or incorporating into the genome of a cell, including an avian, bovine, non-human mammalian, or non-human animal cell, including a fibroblast cell, a nucleic acid encoding PPARG, CEBPA, PAX7, MYOD, MYH2, and/or MEF2B, operably linked to a heat shock promoter. In embodiments, the cells are transfected with a nucleic acid encoding PPARG, CEBPA, PAX7, MYOD, MYH2, and/or MEF2B operably linked to a heat shock promoter. A non-limiting example of a vector which can be used to transfect the cells is shown in FIG. 3. Transfecting a cell with one or more nucleic acids encoding factors or biomarkers associated with a muscle or fat cell phenotype improves the capability of the cell to adopt a muscle or fat cell phenotype. In such cases, the cell line is suited to produce cell types of interest, for example, cell types used for cultured food products (e.g., myotubes and other muscle cell types and fat cell types). In embodiments, the heat shock promoter is a HSP1A1 promoter or HSF-1 promoter.

The methods provided herein include introducing into, or incorporating into the genome of a cell, including a chicken or bovine cell, including a fibroblast cell, a nucleic acid encoding PPARG, CEBPA, PAX7, MYOD, MYH2, and/or MEF2B, operably linked to a heat shock promoter. In embodiments, the cells are transfected with a nucleic acid encoding PPARG, CEBPA, PAX7, MYOD, MYH2, and/or MEF2B operably linked to a heat shock promoter. A non-limiting example of a vector which can be used to transfect the fibroblast cells is shown in FIG. 3. Transfecting a cell with one or more nucleic acids encoding factors or biomarkers associated with a muscle or fat cell phenotype improves the differentiation capability of the cell to adopt a muscle or fat cell phenotype. In such cases, the cell line is suited to produce differentiated cell types of interest, for example, cell types used for cultured food products (e.g., myotubes and other muscle cell types and fat cell types).

In embodiments, the vector construct used to transfect the avian cells comprises: (a) a heat shock promoter and (b) a nucleic acid encoding PPARG, CEBPA, PAX7, MYOD, MYH2, and/or MEF2B operably linked to the HSP1A1 promoter. In some embodiments, the heat shock inducible genes associated with a fat or muscle phenotype and HSP1A1 promoter are derived from avian cells. In embodiments, the heat shock promoter is a HSP1A1 promoter or HSF-1 promoter. In additional embodiments, the HSP1A1 promoter comprises the nucleotide sequence of SEQ ID NO: 29.

In embodiments, the vector construct used to transfect the bovine cells comprises: (a) a heat shock promoter and (b) a nucleic acid encoding PPARG, CEBPA, PAX7, MYOD, MYH2, and/or MEF2B operably linked to the HSP1A1 promoter. In some embodiments, the heat shock inducible genes associated with a fat or muscle phenotype and HSP1A1 promoter are derived from bovine cells. In embodiments, the heat shock promoter is a HSP1A1 promoter or HSF-1 promoter. In additional embodiments, the HSP1A1 promoter comprises the nucleotide sequence of SEQ ID NO: 29.

In embodiments, the vector construct used to transfect the non-human mammalian or non-human animal cells comprises: (a) a heat shock promoter and (b) a nucleic acid encoding PPARG, CEBPA, PAX7, MYOD, MYH2, and/or MEF2B operably linked to the HSP1A1 promoter. In some embodiments, the heat shock inducible genes associated with a fat or muscle phenotype and HSP1A1 promoter are derived from bovine cells. In embodiments, the heat shock promoter is a HSP1A1 promoter or HSF-1 promoter. In additional embodiments, the HSP1A1 promoter comprises the nucleotide sequence of SEQ ID NO: 29.

In further embodiments, the vector construct used to transfect the non-human mammalian or non-human animal cells comprises: (a) a heat shock promoter and (b) a nucleic acid encoding PAX7, MYOD, and MEF2B are co-expressed under the control of a heat shock promoter. In additional embodiments, PAX7, MYOD, and/or MEF2B are induced with a heat shock event. In further embodiments, the vector construct used to transfect the non-human mammalian or non-human animal cells comprises: (a) a heat shock promoter and (b) a nucleic acid encoding MEF2B and MYOD are co-expressed under the control of a heat shock promoter. In additional embodiments, MEF2B and MYOD are induced with a heat shock event. In further embodiments, the vector construct used to transfect the non-human mammalian or non-human animal cells comprises: (a) a heat shock promoter and (b) a nucleic acid encoding MYH2 operably linked to the heat shock promoter. In other embodiments, MYH2 expression is induced with or without a heat shock event. In embodiments, the heat shock promoter is a HSP1A1 promoter or HSF-1 promoter. In additional embodiments, the HSP1A1 promoter comprises the nucleotide sequence of SEQ ID NO: 29.

In embodiments, the cells that are transfected are derived from a cell culture comprising fibroblasts cultured in serum free media.

In embodiments, an avian, bovine, non-human mammalian, or non-human animal cell is transfected with nucleic acids encoding one, two, three, or more factors or biomarkers associated with a muscle or fat cell phenotype. In embodiments, nucleic acids encoding one, two, three, four, or more factors or biomarkers associated with muscle or fat cell phenotype, operably linked to a heat shock promoter, are introduced into or incorporated into the genome of the cell. In embodiments, each additional factor encoded by the nucleic acid transfected, introduced, or incorporated into the cell further improves the capability of the cell to adopt a muscle or fat cell phenotype upon induction. In embodiments where nucleic acids encoding three or more factors are transfected, introduced, or incorporated into a cell, the three or more factor coding sequences are present in one nucleic acid and are operably linked to the same heat shock promoter. In embodiments where nucleic acids encoding three or more factors are transfected, introduced, or incorporated into a cell, two of the factor coding sequences are on different nucleic acids and operably linked to different heat shock promoters. In embodiments, in cases where three or more factors are transfected, introduced, or incorporated into a cell, each of the three factors are encoded by different nucleic acids and operably linked to different heat shock promoters.

In embodiments, the cells are transfected with a nucleic acid encoding PPARG, CEBPA, PAX7, MYOD, MYH2, and MEF2B operably linked to a heat shock promoter using any transfection method known in the art. Non-limiting examples of methods that can be used to transfect the cells include lipid transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral, retroviral, and lentiviral transduction), lipid nanoparticle (LNP) transfection, and nanoparticle transfection. In embodiments, the fibroblast cells are transfected with lipid transfection. In embodiments, the fibroblast cells are transfected with electroporation.

In embodiments, the cells are obtained by transfecting cells with a plasmid comprising a nucleotide sequence encoding PPARG, CEBPA, PAX7, MYOD, MYH2, and MEF2B under control of a heat shock promoter that promotes expression in the cells upon induction by a heat shock.

### 5.1.2.3. Selecting and Culturing Transfected Cells

The methods provided herein include selecting successfully transfected cells and culturing clonally propagating transfected cells. Provided herein is a method of selecting cell lines with an inducible heat shock system, comprising: (a) transfecting avian, bovine, non-human mammalian, or non-human animal cells with a vector construct encoding a fluorescent protein and a heat shock promoter operably linked to a nucleic acid encoding a protein of interest, wherein the fluorescent protein is substantially unintegrated; and (b) selecting cells expressing the fluorescent protein after a first period; and (c) discarding cells expressing the fluorescent protein after a second period, wherein a substantial majority of remaining cells express the nucleic acid operably linked to the heat shock promoter and not the fluorescent protein when cultured at a heat shock temperature.

In embodiments, the nucleic acid construct or vector will include selectable markers to identify successfully transfected cells. In embodiments, the selectable marker is a fluorescent protein, such as GFP or RFP. In embodiments, the selectable marker is transiently expressed episomally and in rare cases is integrated.

In some embodiments, the selectable marker is a fluorescent protein.

In some embodiments, the nucleic acid does not compromise an antibiotic resistance gene. In other embodiments, the vector does include an antibiotic resistance gene, including doxycycline, tetracycline or puromycin resistance gene, and means to excise the antibiotic resistance gene after transfection and, in embodiments, integration into the genome. Such means include a Cre-lox system that can be deployed to excise the antibiotic resistance gene and any other undesirable nucleic acid sequences.

In some embodiments, single cells, including fibroblasts, are isolated and the single cells are grown as clonal cultures. As used herein, “single-cell cloning” refers to the process of isolating an individual cell from a population of cells and subsequently promoting the proliferation of the isolated individual cell to proliferate and establish a uniform community of cells, also referred to as clonal cultures. Clonal cultures derived from single-cell cloning are genetically indistinguishable and form a genetically homogeneous population, which minimizes gene expression variability.

The cells can be isolated into single cells to form a single cell solution using any method known in the art. The cells can be isolated, for example, with serial dilution, microfluidics-based separation, fluorescence activated cell sorting (FACS), single cell droplet dispensing, and filtration. In embodiments, the cells are clonally isolated with serial dilution and seeded into well plates.

In embodiments, the clonal cultures are grown to a cell density of at least 0.1 mil/mL, 0.2 mil/mL, 0.3 mil/mL, 0.4 mil/mL, 0.5 mil/mL, 1 mil/mL, 1.5 mil/mL, 2 mil/mL, 2.5 mil/mL, 3 mil/mL, 4 mil/mL, 5 mil/mL, 6 mil/mL, 7 mil/mL, 8 mil/mL, 9 mil/mL, 10 mil/mL, 11 mil/mL, 12 mil/mL, 13 mil/mL, 14 mil/mL, 15 mil/mL, 16 mil/mL, 17 mil/mL, 18 mil/mL, 19 mil/mL, 20 mil/mL, 21 mil/mL, 22 mil/mL, 23 mil/mL, 24 mil/mL, 25 mil/mL, 26 mil/mL, 27 mil/mL, 28 mil/mL, 29 mil/mL, 30 mil/mL, 31 mil/mL, 32 mil/mL, 33 mil/mL, 34 mil/mL, 35 mil/mL, 36 mil/mL, 37 mil/mL, 38 mil/mL, 39 mil/mL, 40 mil/mL, 41 mil/mL, 42 mil/mL, 43 mil/mL, 44 mil/mL, 45 mil/mL, 46 mil/mL, 47 mil/mL, 48 mil/mL, 49 mil/mL, 50 mil/mL, 51 mil/mL, 52 mil/mL, 53 mil/mL, 54 mil/mL, 55 mil/mL, 56 mil/mL, 57 mil/mL, 58 mil/mL, 59 mil/mL, 60 mil/mL, 61 mil/mL, 62 mil/mL, 63 mil/mL, 64 mil/mL, 65 mil/mL, 66 mil/mL, 67 mil/mL, 68 mil/mL, 69 mil/mL, or at least 70 mil/mL.

In embodiments, the clonal cultures are banked and assessed for phenotype.

In embodiments, the selected clonal cultures do not exhibit substantial leaky expression. Leaky expression is denoted as a basal level of mRNA or protein expression from the heat shock promoter without induction or activation of the heat shock promoter and where the cells are maintained at a temperature that would not activate the heat shock promoter. In embodiments, the expression of one or more factors or biomarkers associated with a fat or muscle phenotype under the control of the heat shock promoters is the same or within 0.1%, 1%, 5% or 10% of levels of the factor in cells not containing the construct when cultured at the recommended culture temperature. Alternatively, the expression in the cells where the expression of the factor is under the control of the heat shock promoter and the cells are cultured at a temperature that does not activate the heat shock promoter is no more than 0.1%, 1%, 5% or 10% of the levels in the cells cultured at a temperature that activates the heat shock promoter and maintains cell viability. In embodiments, the cells comprising the nucleic acid encoding the factor operably linked to the heat shock promoter when cultured at appropriate culture temperature that does not activate the heat shock promoter exhibit doubling times that are the same or within 1%, 5% or 10% of the doubling times of comparable cells that do not comprise the nucleic acid when cultured under the same conditions. In embodiments, the selected cells express the one or more factors or biomarkers associated with a fat or muscle phenotype operably linked to the heat shock promoter to a significant degree when cultured at a heat shock temperature but do not express significant levels of these factors when cultured at a temperature that does not activate the heat shock promoter.

Selecting for transfected cells that do not exhibit significant leaky expression comprises (a) isolating single cells comprising a nucleic acid encoding one or more factors or biomarkers associated with a fat or muscle phenotype operably linked to a heat shock promoter, (b) performing single cell cloning as described previously, (c) measuring expression of the one or more factors or biomarkers associated with a fat or muscle phenotype compared to wild type cells; and (d) culturing cells that only express the one or more factors or biomarkers associated with a fat or muscle phenotype to a significant degree at heat shock temperatures and do not exhibit significant leaky expression at temperatures that do not activate the heat shock promoter. Gradually increasing the temperature to determine the gradient of leaky expression in clonal cultures can also be performed to select for non-leaky clonal cultures. Measuring expression of exogenous genes associated with a fat or muscle phenotype can be performed using the techniques including but not limited to immunoblotting, immunofluorescence, flow cytometry, or other methods.

### 5.1.2.4. Culturing Cells to Activate a Heat Shock Promoter

The methods provided herein include culturing the cells, non-human animal cells, non-human mammalian cells, avian cells, or bovine cells, of the biomass at a temperature that activates the eukaryotic heat shock promoter and maintains cell viability for a time period, wherein the activated eukaryotic heat shock promoter induces expression of one or more nucleic acids encoding factors associated with a fat or muscle phenotype, e.g., a differentiation factor, to promote expression of the fat or muscle phenotype, e.g., differentiation, in the cultured cells.

The methods provided herein also include culturing the cells of the biomass at a heat shock temperature for a time period that activates the heat shock promoter.

In embodiments, the successfully transfected cells are selected and cultured to a desired density. In embodiments, the selected cell lines contain a nucleic acid encoding one or more factors or biomarkers associated with a fat or muscle phenotype operably linked to a heat shock promoter, and optionally, a nucleic acid encoding a heat shock factor.

In embodiments, the heat shock promoter is derived from cattle, pigs, sheep, goats, deer, rabbits, or poultry. In other embodiments, the heat shock promoter is a non-human mammalian HSP70 promoter, a non-human mammalian HSP60 promoter, a non-human mammalian HSP27 promoter, a non-human mammalian HSP100 promoter, a non-human mammalian HSP40 promoter, non-human mammalian a HSP110 promoter, a non-human mammalian HSF-1 promoter, or any other promoter regulated by increased temperature known in the art. In some embodiments, the promoter is a non-human mammalian HSP70 promoter. In some embodiments, the promoter is a non-human mammalian HSF-1 promoter. In other embodiments, the promoter is an avian or a bovine HSP70 promoter or HSF-1 promoter. In additional embodiments, the heat shock promoter comprises the nucleotide sequence of SEQ ID NO: 29. In some embodiments, the one or more genes associated with a fat or muscle phenotype are PPARG, CEBPA, PAX7, MYOD, or MYH2, MEF2B (MERFs). In embodiments, the sequences encoding the factors are from the same species as the cells, for example avian or chicken PPARG, CEBPA, PAX7, MYOD, MYH2, or MEF2B (MERFs) are used in avian or chicken cells. In embodiments, the sequences encoding the factors are non-human or bovine PPARG, CEBPA, PAX7, MYOD, MYH2, or MEF2B (MERFs) are used in non-human or bovine cells. In some embodiments, the heat shock factor is HSP70 or HSF-1.

In some embodiments, one, two, three, or more genes associated with a fat or muscle phenotype are expressed.

In some embodiments, the selected cell lines are clonally derived function cell lines that only express one or more genes associated with a fat or muscle phenotype at a significant level at heat shock temperatures.

In some embodiments, the heat shock temperature to activate expression downstream of the heat shock promoter is at or between 37° C. and 45° C. In embodiments, the temperature is about 37° C., about 38° C., about 39° C., about 40° C., about 41° C., about 42° C., about 43° C., about 44° C., or about 45° C. In further embodiments, the heat shock temperature is between 37° C. and 45° C., between 37° C. and 44° C., between 37° C. and 43° C., between 37° C. and 42° C., between 37° C. and 41° C., between 37° C. and 40° C., between 37° C. and 39° C., or between 37° C. and 38° C. In embodiments, the proliferation temperature range and heat shock temperature range overlap, and the factor or biomarker associated with a fat or muscle phenotype is expressed at a heat shock temperature 3° C. to 7° C. higher than the proliferation temperature. In embodiments, the heat shock temperature is at least 3° C. higher than the proliferation temperature.

The temperatures activate the heat shock promoter while maintaining cell viability, e.g., does not reduce cell viability by more than 1%, 5% or 10% relative to the cell viability at the culture temperature that does not activate the heat shock promoter.

In some embodiments, the cells are cultured at a heat shock temperature for about 1 hour. In embodiments, the cells are cultured at a heat shock temperature for about 1 hour, for about 1.5 hours, for about 2 hours, for about 2.5 hours, for about 3 hours, for about 3.5 hours, for about 4 hours, for about 4.5 hours, for about 5 hours, for about 5.5 hours, for about 6 hours, for about 6.5 hours, for about 7 hours, for about 7.5 hours, for about 8 hours, for about 8.5 hours, for about 9 hours, for about 9.5 hours, for about 10 hours, for about 10.5 hours, for about 11 hours, for about 11.5 hours, for about 12 hours, for about 12.5 hours, for about 13 hours, for about 13.5 hours, for about 14 hours, for about 14.5 hours, for about 15 hours, for about 15.5 hours, for about 16 hours, for about 16.5 hours, for about 17 hours, for about 17.5 hours, for about 18 hours, for about 18.5 hours, for about 19 hours, for about 19.5 hours, for about 20 hours, for about 20.5 hours, for about 21 hours, for about 21.5 hours, for about 22 hours, for about 22.5 hours, for about 23 hours, for about 23.5 hours, for about 24 hours. The period of time is sufficient to activate the heat shock promoter and result in expression of the factors encoded by the nucleic acids operably linked to the heat shock promoter and maintain cell viability. In some embodiments, the transfected cells have a basal doubling time before exposure to the heat shock temperature, wherein the cells return to the basal doubling time within 6 days of exposure to the heat shock temperature. In some embodiments, the cell line returns to a doubling time of less than 24 hours 6 days or less after exposure to a heat shock temperature for a time length between about 1 and 6 hours. In other embodiments, the doubling time of less than 24 hours returns after 6 days or less, after 5 days or less, after 3 days or less, after 2 days or less after 1 day or less after exposure to a heat shock temperature for a time length between about 1 and 6 hours. In other embodiments, the doubling time of less than 24 hours is 23.5 hours, 23 hours, 22.5 hours, hours, 22 hours, 21.5 hours, 21 hours, 20.5 hours, 20 hours, 19.5 hours, 19 hours, 18.5 hours, 18 hours, 17.5 hours, 17 hours, 16.5 hours, 16 hours, 15.5 hours, 15 hours, 14.5 hours, 14 hours, 13.5 hours, 13 hours, 12.5 hours, 12 hours, 11.5 hours, 11 hours, 10.5 hours, 10 hours, 9.5 hours, 9 hours, 8.5 hours, 8 hours, 7.5 hours, 7 hours, 6.5 hours, 6 hours, 5.5 hours, 5 hours, 4.5 hours, 4 hours, 3.5 hours, 3 hours, 2.5 hours, 2 hours, 1.5 hours, or 1 hour.

In some embodiments, once the selected cells have been cultured at a heat shock temperature for a time period that activates the heat shock promoter, the cellular energy is redirected away from proliferation towards expression of factors or biomarkers associated with fat or muscle phenotypes, e.g. differentiation or development of a fat or muscle cell phenotype. In some embodiments, cells exposed to a time period of heat shock may be further cultured at temperatures conducive to differentiation (which is typically the same temperature as is conducive to proliferation) for 1 to 14 days, i.e., a finishing time period, during which time the cells expression of factors or biomarkers associated with fat or muscle phenotypes has time to develop further, e.g., progressively increased expression of such phenotypes, and in some instances the cells may begin to resemble myogenic or adipogenic cells. In embodiments, the finishing time period is for 1 to 14 days, 1 to 13 days, 1 to 12 days, 1 to 11 days, 1 to 10 days, 1 to 9 days, 1 to 8 days, 1 to 7 days, 1 to 6 days, 1 to 5 days, 1 to 4 days, 1 to 3 days, 1 to 2 days, 2 to 14 days, 2 to 13 days, 2 to 12 days, 2 to 11 days, 2 to 10 days, 2 to 9 days, 2 to 8 days, 2 to 7 days, 2 to 6 days, 2 to 5 days, 2 to 4 days, 2 to 3 days, 3 to 14 days, 3 to 13 days, 3 to 12 days, 3 to 11 days, 3 to 10 days, 3 to 9 days, 3 to 8 days, 3 to 7 days, 3 to 6 days, 3 to 5 days, 3 to 4 days, 4 to 14 days, 4 to 13 days, 4 to 12 days, 4 to 11 days, 4 to 10 days, 4 to 9 days, 4 to 8 days, 4 to 7 days, 4 to 6 days, 4 to 5 days, 5 to 14 days, 5 to 13 days, 5 to 12 days, 5 to 11 days, 5 to 10 days, 5 to 9 days, 5 to 8 days, 5 to 7 days, 5 to 6 days, 6 to 14 days, 6 to 13 days, 6 to 12 days, 6 to 11 days, 6 to 10 days, 6 to 9 days, 6 to 8 days, 6 to 7 days, 7 to 14 days, 7 to 13 days, 7 to 12 days, 7 to 11 days, 7 to 10 days, 7 to 9 days, 7 to 8 days, 8 to 14 days, 8 to 13 days, 8 to 12 days, 8 to 11 days, 8 to 10 days, 8 to 9 days, 9 to 14 days, 9 to 13 days, 9 to 12 days, 9 to 11 days, 9 to 10 days, 10 to 14 days, 10 to 13 days, 10 to 12 days, 10 to 11 days, 11 to 14 days, 11 to 13 days, 11 to 12 days, 12 to 14 days, 12 to 13 days, or 13 to 14 days.

In embodiments, prior to clonally isolating the non-human animal, non-human mammalian, avian, chicken or bovine cells, including fibroblast cells or stem cells, the cells are in suspension, suspension aggregates, adherent cell culture, or microcarriers. The cells have grown to at least 0.1 mil/mL, 0.2 mil/mL, 0.3 mil/mL, 0.4 mil/mL, 0.5 mil/mL, 1 mil/mL, 1.5 mil/mL, 2 mil/mL, 2.5 mil/mL, 3 mil/mL, 4 mil/mL, 5 mil/mL, 6 mil/mL, 7 mil/mL, 8 mil/mL, 9 mil/mL, 10 mil/mL, 11 mil/mL, 12 mil/mL, 13 mil/mL, 14 mil/mL, 15 mil/mL, 16 mil/mL, 17 mil/mL, 18 mil/mL, 19 mil/mL, 20 mil/mL, 21 mil/mL, 22 mil/mL, 23 mil/mL, 24 mil/mL, 25 mil/mL, 26 mil/mL, 27 mil/mL, 28 mil/mL, 29 mil/mL, 30 mil/mL, 31 mil/mL, 32 mil/mL, 33 mil/mL, 34 mil/mL, 35 mil/mL, 36 mil/mL, 37 mil/mL, 38 mil/mL, 39 mil/mL, 40 mil/mL, 41 mil/mL, 42 mil/mL, 43 mil/mL, 44 mil/mL, 45 mil/mL, 46 mil/mL, 47 mil/mL, 48 mil/mL, 49 mil/mL, 50 mil/mL, 51 mil/mL, 52 mil/mL, 53 mil/mL, 54 mil/mL, 55 mil/mL, 56 mil/mL, 57 mil/mL, 58 mil/mL, 59 mil/mL, 60 mil/mL, 61 mil/mL, 62 mil/mL, 63 mil/mL, 64 mil/mL, 65 mil/mL, 66 mil/mL, 67 mil/mL, 68 mil/mL, 69 mil/mL, or at least about 70 mil/mL in serum free media.

In terms of scale, the cells are cultured in the vessels configured to hold a suspension culture volume that ranges from about 10 L to about 500,000 L. In embodiments, the vessel can hold a volume of cell culture that is about 50 mL, about 100 mL, about 500 mL, about 1 L, about 10 L, about 100 L, about 1000 L, about 3,000 L, about 5,000 L, about 10,000 L, about 25,000 L, about 50,000 L, about 100,000 L, about 200,000 L, about 250,000 L, or about 500,000 L. The culture vessel can hold a volume of suspension culture, for example and without limitation, from about 50 mL to about 100 mL, about 50 mL to about 500 mL, about 50 mL to about 1 L, about 50 mL to about 5 L, about 50 mL to about 20 L, about 50 mL to about 100 L, about 50 mL to about 500 L, about 50 mL to about 10,000 L, about 50 mL to about 50,000 L, about 50 mL to about 100,000 L, about 50 mL to about 500,000 L, about 100 mL to about 500 mL, about 100 mL to about 1 L, about 100 mL to about 5 L, about 100 mL to about 10 L, about 100 mL to about 50 L, about 100 mL to about 100 L, about 100 mL to about 250 L, about 100 mL to about 1000 L, about 100 mL to about 10,000 L, about 100 mL to about 100,000 L, about 100 mL to about 500,000 L, about 500 mL to about 1 L, about 500 mL to about 5 L, about 500 mL to about 10 L, about 500 mL to about 50 L, about 500 mL to about 100 L, about 500 mL to about 250 L, about 500 mL to about 1000 L, about 500 mL to about 10,000 L, about 500 mL to about 100,000 L, about 500 mL to about 500,000 L, about 1 L to about 10 L, from about 1 L to about 50 L, from about 1 L to about 100 L, from about 10 L to about 100 L, from about 10 L to about 200 L, from about 10 L to about 350 L, from about 10 L to about 500 L, from about 10 L to about 1000 L, from about 50 L to about 250 L, from about 50 L to about 500 L, from about 50 L to about 1000 L, from about 100 L to about 500 L, from about 100 L to about 1000 L, from about 100 L to about 2,500 L, from about 100 L to about 3,000 L, from about 100 L to about 10,000 L, from about 500 L to about 1000 L, from about 500 L to about 2,500 L, from about 500 L to about 5,000 L, from about 500 L to about 10,000 L, from about 500 L to about 25,000 L, from about 1,000 L to about 1,500 L, from about 1,000 L to about 5,000 L, from about 1,000 L to about 10,000 L, from about 1,000 L to about 50,000 L, from about 1,000 L to about 100,000 L, from about 5,000 L to about 10,000 L, from about 5,000 L to about 25,000 L, from about 5,000 L to about 50,000 L, from about 5,000 L to about 100,000 L, from about 5,000 L to about 250,000 L, from about 10,000 L to about 50,000 L, from about 10,000 L to about 100,000 L, from about 10,000 L to about 250,000 L, from about 10,000 L to about 500,000 L, from about 25,000 L to about 50,000 L, from about 25,000 L to about 100,000 L, from about 25,000 L to about 250,000 L, from about 25,000 L to about 500,000 L, from about 50,000 L to about 100,000 L, from about 50,000 L to about 250,000 L, from about 50,000 L to about 500,000 L, from about 100,000 L to about 250,000 L, from about 100,000 L to about 500,000 L, from about 200,000 L to about 250,000 L, from about 200,000 L to about 500,000 L, or from about 250,000 L to about 500,000 L.

In embodiments, the expression of one or more factors or biomarkers associated with a fat or muscle phenotype is higher in heat shocked cells than non-transfected cells or cells cultured at a standard temperature for a time. In further embodiments, the expression of one or more biomarkers associated with a fat or muscle phenotype is substantially higher in heat shocked cells than non-transfected cells or cells cultured at a standard temperature for a time. In some embodiments, the expression of one or more biomarkers associated with a fat or muscle phenotype is at 0.5 to 30 fold higher in heat shocked cells than non-transfected cells or cells cultured at a standard temperature for a time. In other embodiments, the expression of the expression of one or more biomarkers associated with a fat or muscle phenotype is 0.5 fold, 1 fold, 2 fold, 3 fold, 4 fold, 5 fold, 6 fold, 7 fold, 8 fold, 9 fold, 10 fold, 11 fold, 12 fold, 13 fold, 14 fold, 15 fold, 16 fold, 17 fold, 18 fold, 19 fold, 20 fold, 21 fold, 22 fold, 23 fold, 24 fold, 25 fold, 26 fold, 27 fold, 28 fold, 29 fold or 30 fold higher in heat shocked cells than non-transfected cells or cells cultured at a standard temperature for a time.

In some embodiments, upon induction of expression of the factors through activation of the heat shock promoter, the cells exhibit phenotypes of adipocytes or myotubes. In embodiments, the cells can also differentiate into mature cells, including but not limited to adipocytes, such as white adipocytes, brown adipocytes, and beige adipocytes, or muscle cells, such as myocytes, myoblasts, myotubes, multinucleated myotubes, satellite cells, and skeletal muscle cells. In other embodiments, the cells are a mixture of adipocytes and muscle cells. In further embodiments, the mixture of cells includes but is not limited to white adipocytes, brown adipocytes, beige adipocytes, myocytes, myoblasts, myotubes, multinucleated myotubes, satellite cells, or skeletal muscle cells.

In some embodiments, the cells are cultured in serum free media. Serum free media refers to media that is substantially or virtually free of serum. In some embodiments, the cell culture media is hormone free. In some embodiments, the cell culture media is heavy metal free. In some embodiments, the cell culture media is antibiotic free. In other embodiments, the cell culture media is animal-component free.

In embodiments, the cellular population exhibits less than 0.1%, 1%, or 10% expression of the one or more genes associated with a fat or muscle phenotype operably linked to the heat shock promoter at or below 37° C. relative to expression in comparable cells exposed to temperatures that activate the heat shock promoter or relative to wild type cells.

In embodiments, the cellular population exhibits less than 10% expression of the one or more genes associated with a fat or muscle phenotype operably linked to the heat shock promoter at or below 37° C. relative to expression in comparable cells exposed to temperatures that activate the heat shock promoter or relative to wild type cells.

In embodiments as described herein, the cells used in the methods of the invention are from a cell line in which the cells comprise one or more nucleic acids encoding one or more genes associated with a fat or muscle phenotype operably linked to a heat shock promoter. In embodiments, the nucleic acids encoding the one or more genes associated with a fat or muscle phenotype are episomal. In embodiments, the nucleic acids encoding the one or more genes associated with a fat or muscle phenotype are integrated into the genome of the cells of the cell line. In embodiments, the one or more genes associated with a fat or muscle phenotype are PPARG, CEBPA, PAX7, MYOD, MYH2, and MEF2B. In embodiments, the one or more genes associated with a fat or muscle phenotype is from the same species as the cultured cells. Amino acid sequences of PPARG, PAX7, MYOD and MEF2B from different species, including chicken, duck, turkey, pig, and bovine (beef) are provided in Table 1 and Table 2. In embodiments, the PPARG, MYOD, PAX7, and MEF2B are chicken PPARG, MYOD, PAX7, and MEF2B and, in embodiments, have amino acid sequences of SEQ ID NO: 19, SEQ ID NO: 1, SEQ ID NO: 7, and SEQ ID NO: 13, respectively. In embodiments, the PPARG, MYOD, PAX7, and MEF2B are bovine PPARG, MYOD, PAX7 and MEF2B and, in embodiments, have amino acid sequences of SEQ ID NO: 23, SEQ ID NO: 5, SEQ ID NO: 11, SEQ ID NO: 17, SEQ ID NO: 26, and SEQ ID NO: 28, respectively. In embodiments, the nucleic acids encoding these factors are operably linked and under control of the heat shock promoter elements. In embodiments, the nucleic acids encoding these factors are independent of each other.

### 5.1.2.5. Preparation for Consumption

The methods provided herein include methods for producing a cultured cell-based product for dietary consumption, comprising: (a) transfecting cells, comprising avian, bovine, non-human mammalian, or non-human animal cells, with a nucleic acid encoding a factor or biomarker associated with a fat or muscle phenotype operably linked to a heat shock promoter; (b) selecting successfully transfected cells; (c) culturing the transfected cells at a heat shock temperature that activates the heat shock promoter and maintains cell viability for a time period, wherein the activated heat shock promoter induces expression of the fat or muscle phenotype; and (d) obtaining and processing the cells expressing the fat or muscle phenotype for consumption.

In embodiments, the cultured cell-based meat compositions or cultured cell-based food products comprising avian, bovine, or non-human cultured cells induced to express one or more genes associated with a fat or muscle phenotype. In some embodiments, the cells are combined with other ingredients to make a cell-based meat composition or cell-based food product (e.g., a patty, a nugget, a sausage, or a tender). In embodiments, the cells are combined with an emulsion mixture to make a cell-based meat product (e.g., a patty, a nugget, a sausage, or a tender).

The cell-based product meets standards for food safety required by government regulation. In embodiments, the cell-based product meets standards for food safety required by the U.S. Food and Drug Administration (FDA), the U.S. Department of Agriculture (USDA), the European Food Safety Authority, or other state or regional food regulatory agencies. In embodiments, the cell-based product includes ingredients that meet standards for food safety required by the FDA, USDA, and European Food Safety Authority. In embodiments, the cell-based product may comprise ingredients approved for use in food that are recognized by the FDA as Generally Recognized As Safe (GRAS). In embodiments, the cell-based product comprises ingredients that are GRAS certified ingredients, non-GRAS certified ingredients, or mixtures thereof. In embodiments, the cell-based product comprises only GRAS certified ingredients. As the list of GRAS substances is updated by the FDA, the GRAS certified ingredients that may be used in the cell-based meat composition may be modified.

In embodiments, cells are differentiated into adipocytes or myotubes.

In embodiments, the final product includes a final cellular product that includes heterogeneous population of cells.

### 5.2. Characteristics of Cell Line

The methods and compositions provided herein include a chicken (or other avian) or bovine (or other non-human animal or non-human mammalian cell line) cell line, i.e. a fibroblast cell line, which comprises a nucleic acid encoding one or more factors or biomarkers associated with a fat or muscle phenotype operably linked to a heat shock promoter, said cell line characterized by: suspension adaptation, a population doubling level exceeding 100, a doubling time less than 24 hours, a culture density tolerance exceeding 1×10⁷ cells/mL, adaptation to serum free media, edibility, and heat shock inducible gene expression.

The methods and compositions provided herein include described chicken or bovine cell lines, including fibroblast cell lines, which are functionally immortalized such that they can be cultured to at least one hundred doublings.

The methods provided herein include disclosed methods of producing a cell-based product for dietary consumption or a method of selecting cell lines with heat shock inducible nucleic acids encoding factors or biomarkers associated with a fat or muscle phenotype, wherein the avian, bovine, non-human mammalian, or non-human animal cells are or have been transfected with a construct comprising: (a) a heat inducible heat shock promoter and (b) a nucleic acid encoding one or more factors or biomarkers associated with a fat or muscle phenotype operably linked to the heat shock promoter.

The methods provided herein include producing a cell line with heat shock inducible nucleic acids encoding factors, comprising: (a) transfecting cells, comprising avian, bovine, non-human mammalian, or non-human animal cells with a vector construct comprising a nucleic acid encoding a factor operably linked to a heat shock promoter; (b) discarding cells that express the heat shock promoter linked nucleic acid at proliferation temperatures, wherein remaining cells are cultured further; and (c) culturing the remaining cells at a heat shock temperature for a time period that activates the heat shock promoter, wherein less than 10% of the remaining cells express the heat shock promoter-linked nucleic acid at or below 37° C.

The methods provided herein include methods of producing a cell-based product for dietary consumption or a method of selecting cell lines with heat shock inducible genes associated with a fat or muscle phenotype, wherein the avian, bovine, non-human mammalian, or non-human animal cells have been or are transfected with a construct comprising: (a) a heat inducible heat shock promoter, (b) a nucleic acid encoding one or more factors or biomarkers associated with a fat or muscle phenotype operably linked to the heat shock promoter, and (c) a nucleic acid encoding a heat shock factor, wherein expression of the heat shock factor and the one or more factors or biomarkers associated with a fat or muscle phenotype are induced at a heat shock temperature, and the heat shock factor continuously activates the heat shock promoter to increase expression of the one or more genes associated with a fat or muscle phenotype.

Also provided herein are methods for producing a cell based product for dietary consumption, the method comprising: culturing avian or bovine cells at a first temperature for a first time, which cells comprise a nucleic acid encoding a factor or biomarker associated with a fat or muscle phenotype operably linked to a heat shock promoter (including either transiently transfected cells or those stably incorporating such a nucleic acid), such that the cells proliferate to form a biomass but do not express the phenotype to a significant degree at a first temperature (e.g., a proliferation temperature) for a first time; culturing the cells of the biomass at a second temperature (i.e., a heat shock temperature) for a second time that activates the eukaryotic heat shock promoter and maintains cell viability, wherein, at the second temperature, the heat shock promoter induces expression of one or more factors or biomarkers associated with a fat or muscle phenotype, e.g. a differentiation factor to promote differentiation of the cells or a biomarker that is associated with differentiation; and obtaining and processing the induced cells of the biomass for consumption. In embodiments, the avian, bovine, non-human mammalian, or non-human animal cells have been transfected with a nucleic acid encoding a factor or biomarker associated with a fat or muscle phenotype operably linked to a heat shock promoter; successfully transfected cells are selected and either grown polyclonally or selected and then cloned and cultured monoclonally. Clonal selection allows isolation of a cell line that does not exhibit significant leaky expression of the one or more factors or biomarkers associated with a fat or muscle phenotype when not induced by heat shock but expresses such factors when exposed to the heat shock temperature, including at a level that can promote expression of muscle or fat cell phenotype.

The compositions provided herein include an edible avian, bovine, non-human mammalian, or non-human animal cell line comprising: (a) inducible gene expression when cultured at temperatures of 43° C.

The compositions provided herein include an edible avian, bovine, non-human mammalian, or non-human animal cell line wherein the inducible gene expression comprises an integrated heat shock promoter operably linked to one or more factors (e.g. genes) associated with a fat or muscle phenotype.

The compositions provided herein include an edible avian, bovine, non-human mammalian, or non-human animal cell line wherein the inducible gene expression comprises an integrated heat shock promoter factor.

The compositions provided herein include an edible avian, bovine, non-human mammalian, or non-human animal cell line wherein the integrated heat shock factor is operably linked to a heterologous or homologous heat shock promoter or an integrated heat shock promoter, and heat activation induces a positive feedback loop of expression.

In embodiments, the cell line returns to a doubling time of less than 24 hours 6 days or less after exposure to a heat shock temperature for a time length between about 1 and 6 hours. In other embodiments, heat shock inducible gene expression is induced at a temperature at or between 37° C. and 43° C. In some embodiments, the inducible gene expression comprises one or more genes associated with a fat or muscle phenotype operably linked to a heat shock promoter. In additional embodiments, the heat shock promoter is a portion of the heat shock promoter having heat-inducible promoter activity. In even further embodiments, the heat shock promoter is modified to exhibit reduced activation by heavy metals or ultraviolet light as compared to a native heat shock promoter. In embodiments, the factor or biomarker associated with a fat phenotype is PPARG and/or CEBPA or the factor or biomarker associated with a muscle phenotype is PAX7, MYOD, MYH2 and/or MEF2B (MERFs).

### 5.2.1. Suspension Adaptation

In some embodiments, the cells are grown in any vessel suitable for suspension culture. Non-limiting examples of suitable vessels include tubes, flasks, multi-well plates, dishes, vats, bioreactors, and the like.

In some embodiments, the cells are non-myogenic, and such non-myogenic cells can be programmed to be myogenic, for example the cells may comprise fibroblasts, stem cells, or other cell type modified to express one or more factors or biomarkers associated with muscle phenotype under the control of a heat shock promoter. In some embodiments, the myogenic factors include MYOD1, MYOG, MYF5, MYF6, PAX3, PAX7, MYH2, paralogs, orthologs, and genetic variants thereof. In some embodiments, the cells are modified to express one or more myogenic transcription factors as described in a PCT publication, WO/2015/066377, which is herein incorporated by reference in its entirety.

In some embodiments, the cells are genetically modified to inhibit a pathway, e.g. the HIPPO signaling pathway. Exemplary methods to inhibit the HIPPO signaling pathway as described in a PCT Application No. PCT/US2018/031276, which is herein incorporated by reference in its entirety.

In some embodiments, the cells are modified to express glutamine synthetase (GS), insulin-like growth factor (IGF), and/or albumin. Exemplary methods of modifying cells to express GS, IGF, and/or albumin are described in a PCT Application No. PCT/US2018/042187 which is herein incorporated by reference in its entirety.

In some embodiments, anchorage-dependent cells can be adapted for cultivation in suspension, and can include at least the following steps: (1) transfect an anchorage-dependent, chicken or bovine cell with a construct expressing YAPl or TAZ under the control of an inducible eukaryotic promoter, including a heat shock promoter or doxycycline inducible promoter, and, in embodiments, containing an antibiotic-resistance gene; (2) select and enrich the transfected cells, including, for example, with a dosage of antibiotic lethal to the non-transfected cells; (3) passage the cells in a single-cell suspension to a shaker flask in proliferation medium supplemented with doxycycline, and shake flask in a slow, orbital motion sufficient to prevent the cells from sedimentation within the flask and keep cells in suspension; (4) clonally isolate proliferating cells from the suspension culture and passage to another suspension culture, as described in a PCT publication, WO 2018/208628, which is herein incorporated by reference in its entirety.

### 5.2.2. Functional Immortality

In some embodiments, the cells are modified to express telomerase reverse transcriptase (TERT) and/or inhibit cyclin-dependent kinase inhibitors (CKI). In some embodiments, the cells are modified to express TERT and/or inhibit cyclin-dependent kinase inhibitors as described in a PCT publication, WO 2017/124100, which is herein incorporated by reference in its entirety.

Telomerase expression plays a role in cellular senescence, as it is normally repressed in postnatal somatic cells resulting in progressive shortening of telomeres. Cell lines expressing TERT have shown functional immortality by achieving 100+ doublings without senescence. In some examples, cell lines expressing TERT have been proliferated to over 300+ doublings without senescence.

In some embodiments, immortalization comprises transforming, introducing, or incorporating into the genome of a cell nucleic acid sequence encoding a telomerase reverse transcriptase (TERT) gene. In some embodiments, cells ectopically express the TERT polynucleotide. In some embodiments, the cells are genetically modified and carry stable integrations of one or more copies of the TERT polynucleotide.

In some embodiments, increased expression of TERT may be achieved using different approaches. In some embodiments, increased expression of TERT may be achieved by ectopically expressing TERT. In some embodiments, increased expression of TERT may be achieved by introducing targeted mutations in the native TERT promoter. In some embodiments, increased expression of TERT may be achieved by activating endogenous TERT expression by an engineered transcriptional activator. In some embodiments, increased expression of TERT may be achieved by transiently transfecting TERT mRNA.

The polynucleotide encoding TERT can be from any organism. The TERT polynucleotide can be from bacteria, plants, fungi, and archaea. The TERT polynucleotide can be from any animal, such as vertebrate and invertebrate animal species. The TERT polynucleotide can be from any vertebrate animal species such as mammals, reptiles, birds, amphibians, and the like. The TERT polynucleotide can be from any non-human mammal or non-human animal species, such as a murine, bovine, porcine, and the like.

### 5.2.3. Doubling Time

In some embodiments, prior to inducing expression of the factor by heat shock the cell line or immortalized cell line had a population doubling level (PDL) of at least 60 (e.g., at least 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more passages). In some embodiments, cells including a nucleic acid encoding one or more genes associated with a fat or muscle phenotype exhibit a doubling time within 1%, 5%, or 10% of a control lacking integration of the one or more genes associated with a fat or muscle phenotype, even when such genes result in detectable levels of leaky expression at proliferation temperatures.

In some embodiments, the doubling times of cells at proliferation temperatures are not affected, for example, by an increase in double time that is no more than 1%, 5%, 10%, 15%, 20%, 25%, 30% or 33% longer than control cells, by incorporating a nucleic acid encoding one or more factors or biomarkers associated with a fat or muscle phenotype operably linked to a heat shock promoter.

In some embodiments, the transfected cells have a basal doubling time before exposure to the heat shock temperature, wherein the cells return to the basal doubling time within 6 days of exposure to the heat shock temperature. In some embodiments, the cell line returns to a doubling time of less than 24 hours 6 days or less after exposure to a heat shock temperature for a time length between about 1 and 6 hours. In other embodiments, the doubling time of less than 24 hours returns after 6 days or less, after 5 days or less, after 3 days or less, after 2 days or less after 1 day or less after exposure to a heat shock temperature for a time length between about 1 and 6 hours. In other embodiments, the doubling time of less than 24 hours is 23.5 hours, 23 hours, 22.5 hours, hours, 22 hours, 21.5 hours, 21 hours, 20.5 hours, 20 hours, 19.5 hours, 19 hours, 18.5 hours, 18 hours, 17.5 hours, 17 hours, 16.5 hours, 16 hours, 15.5 hours, 15 hours, 14.5 hours, 14 hours, 13.5 hours, 13 hours, 12.5 hours, 12 hours, 11.5 hours, 11 hours, 10.5 hours, 10 hours, 9.5 hours, 9 hours, 8.5 hours, 8 hours, 7.5 hours, 7 hours, 6.5 hours, 6 hours, 5.5 hours, 5 hours, 4.5 hours, 4 hours, 3.5 hours, 3 hours, 2.5 hours, 2 hours, 1.5 hours, or 1 hour.

In some embodiments, the cell line returns to a doubling time of less than 24 hours 6 days or less after exposure to a heat shock temperature for a time length between about 1 and 6 hours. In other embodiments, the doubling time of less than 24 hours returns after 6 days or less, after 5 days or less, after 3 days or less, after 2 days or less after 1 day or less after exposure to a heat shock temperature for a time length between about 1 and 6 hours. In other embodiments, the doubling time of less than 24 hours is 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, or 1 hour.

### 5.2.4. Culture Density

The culture density of cells may reach about 10⁵ cells/mL, about 10⁶ cells/mL, about 10⁷ cells/mL, about 10⁸ cells/mL, about 10⁹ cells/mL, or about 10¹⁰ cells/mL (cells in the cellular biomass/mL of cultivation infrastructure), including values and ranges there between.

In some embodiments, increasing the cell density of a culture comprising of the cell line comprises introducing any combination of the following cellular modifications: increased expression of GS, increased expression of IGF, increased expression of albumin, increased expression of telomerase reverse transcriptase (TERT), loss-of-function mutations in cyclin-dependent kinase inhibitor (CKI) proteins, increased expression of YAP, increased expression of TAZ, increased expression of myogenic transcription factors.

In some embodiments, increasing the cell density of the avian, chicken, bovine, non-human mammalian, non-human animal cell line, including fibroblast, stem cell, or other cell type, comprises (a) introducing into the cells one or more polynucleotide sequences encoding glutamine synthetase (GS), insulin-like growth factor (IGF), and albumin; and (b) culturing the cells in a cultivation infrastructure.

In some embodiments, increasing the cell density of the avian, chicken, bovine, non-human mammalian, non-human animal cell line comprises (a) introducing into the cells one or more polynucleotide sequences encoding glutamine synthetase (GS), insulin-like growth factor (IGF), albumin or combinations (GS+IGF; GS+albumin; IGF+albumin; GS+IGF+albumin) thereof; and (b) culturing the cells in a cultivation infrastructure.

In some embodiments, increasing the cell density of the avian, chicken, bovine, non-human mammalian, non-human animal cell line comprises (a) introducing into the cells one or more polynucleotide sequences encoding glutamine synthetase (GS), insulin-like growth factor (IGF), albumin or combinations (GS+IGF; GS+albumin; IGF+albumin; GS+IGF+albumin) thereof; (b) introducing into the cells a polynucleotide sequence encoding a telomerase reverse transcriptase (TERT); and (c) culturing the cells expressing GS, IGF, albumin, or combinations thereof and TERT in a cultivation infrastructure. The previous embodiments described herein are described in a PCT publication, WO 2019/014652, which is herein incorporated by reference in its entirety.

In embodiments, the clonal cultures are grown to a cell density, including prior to induction by heat shock of expression, of at least 0.1 mil/mL, 0.2 mil/mL, 0.3 mil/mL, 0.4 mil/mL, 0.5 mil/mL, 1 mil/mL, 1.5 mil/mL, 2 mil/mL, 2.5 mil/mL, 3 mil/mL, 4 mil/mL, 5 mil/mL, 6 mil/mL, 7 mil/mL, 8 mil/mL, 9 mil/mL, 10 mil/mL, 11 mil/mL, 12 mil/mL, 13 mil/mL, 14 mil/mL, 15 mil/mL, 16 mil/mL, 17 mil/mL, 18 mil/mL, 19 mil/mL, 20 mil/mL, 21 mil/mL, 22 mil/mL, 23 mil/mL, 24 mil/mL, 25 mil/mL, 26 mil/mL, 27 mil/mL, 28 mil/mL, 29 mil/mL, 30 mil/mL, 31 mil/mL, 32 mil/mL, 33 mil/mL, 34 mil/mL, 35 mil/mL, 36 mil/mL, 37 mil/mL, 38 mil/mL, 39 mil/mL, 40 mil/mL, 41 mil/mL, 42 mil/mL, 43 mil/mL, 44 mil/mL, 45 mil/mL, 46 mil/mL, 47 mil/mL, 48 mil/mL, 49 mil/mL, 50 mil/mL, 51 mil/mL, 52 mil/mL, 53 mil/mL, 54 mil/mL, 55 mil/mL, 56 mil/mL, 57 mil/mL, 58 mil/mL, 59 mil/mL, 60 mil/mL, 61 mil/mL, 62 mil/mL, 63 mil/mL, 64 mil/mL, 65 mil/mL, 66 mil/mL, 67 mil/mL, 68 mil/mL, 69 mil/mL, or at least 70 mil/mL. In embodiments, cell densities are kept between 0.1 mil/mL and 5 mil/mL during cell line development and cell line expansion, e.g. for gene editing or for expanding cultures from smaller to larger culture vessels. In embodiments, cell densities are raised to between 6 mil/mL and 70 mil/mL, e.g. during production runs to increase harvestable biomass.

### 5.2.5. Adaptation to Serum Free Media

In embodiments, the cells are adapted to suspension culture and serum free media. In embodiments, the cells are adapted to suspension culture. In embodiments, the cells are adapted to serum free media.

In embodiments, the cells are cultured in a gradient cultivation structure, wherein a concentration of the serum in the media is reduced over time to adapt the cells to a serum free media regimen.

In some embodiments, the cell culture medium comprises non-animal derived serum. Examples of non-animal derived serum include synthetic serum, serum substitutes, serum replacements, chemically defined serum, KnockOut™ Serum Replacement, and SigMatrix Serum diluent. In some embodiments, the cell culture medium comprises no more than 10%, 9%, 8%, 7%, 6%, 5%, 4%, 3%, 2%, 1%, 0.5%, 0.1%, 0.05%, or 0.01% non-animal derived serum. In some embodiments, the cell culture medium is substantially free of non-animal derived serum. Substantially free refers to an amount of non-animal derived serum present at or below level of detection. For example, the level of detection may be determined by mass spectrometry-based techniques, PCR-based techniques, antibody-based techniques, ELISA-based techniques, HPLC-based techniques, and other well-known chemical or biological detection methods. In some embodiments, the cell culture medium is free of non-animal derived serum.

### 5.2.6. Edibility

In some embodiments, the method of making a cell-based meat suitable for consumption comprises forming the cell-based meat composition into a patty (or sausage, nugget, or tender). In some embodiments, producing the cell-based meat composition from the hydrated textured subcomponent and the emulsion mixture (and optionally the dry blend) may require mechanical energy (e.g., blending, vortexing, homogenization, agitation, sonication, high pressure, or any other suitable mechanical activity).

In some embodiments, the cell-based meat products produced exhibit a moisture content and/or texture profile (e.g., hardness) unique from other cell-based meat compositions. For example, in some embodiments, the method of making a cell-based meat suitable for consumption comprises forming the cell-based meat composition into a patty (or sausage, nugget, or tender) having a water activity (aw) of about 0.7 to about 0.9; a hardness of between about 1000 grams (g) and about 6000 g; a resilience score from a TPA analysis of between about 4.0 and 9.0; a cohesiveness score from a TPA analysis of between about 0.1 to about 0.8; a springiness score from a TPA analysis of between about 20 to about 80; a gumminess score from a TPA analysis of between about 100 to about 1000; a chewiness score from a TPA analysis of between about 100 to about 500; and any of the binding scores described herein, or a combination thereof.

### 5.3. Draw and Fill Systems and Methods

Provided herein is a draw and fill method comprising cultivating the cells obtained from the methods described herein and comprising the nucleic acids described herein in a bioreactor having a capacity of at least 20 kiloliters until reaching a packed cell volume (PCV) of at least 6%; draining at least 60% of the cell culture to a differentiation system; culturing the cells in the differentiation system to an average temperature of 43° C. to induce expression of at least one factor or biomarker associated with a fat or muscle phenotype, e.g. differentiation, in a substantial portion of the cells; removing cell culture media from the cells; and harvesting the cells for formulation into a comestible food product.

Provided herein is a draw and fill method, wherein the differentiation system is a tank the cells cultured in or a pipe the cells flow through.

The cultivation step can be completed as described herein.

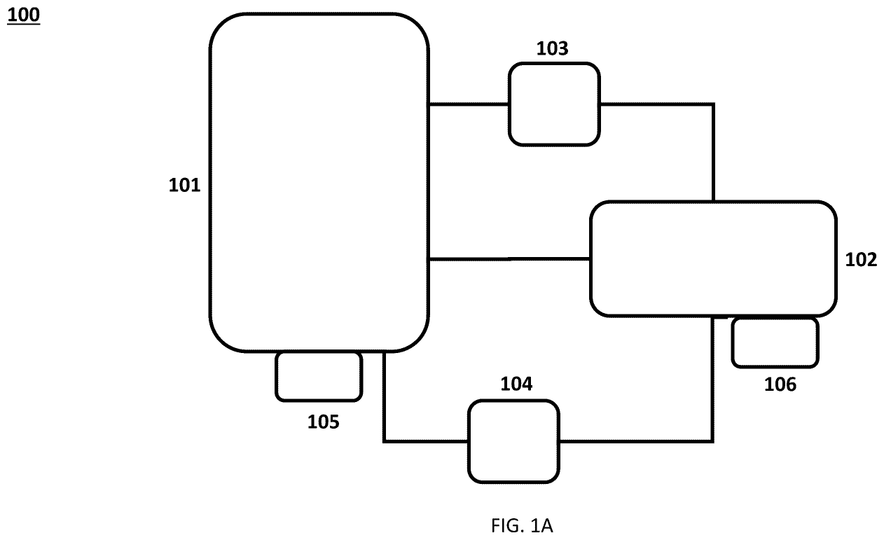

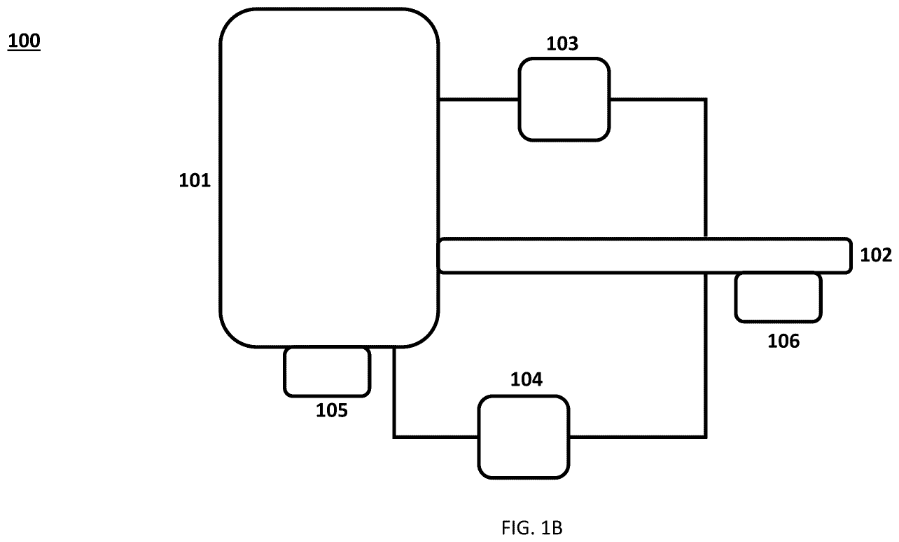

A system configured to perform the disclosed method is shown in FIG. 1A. The system comprises a bioreactor 101, configured to culture and expand cells, a differentiation system 102, configured to culture cells at a heat shock temperature, an aerator 105, configured to percolate oxygen and other gasses, and a centrifuge 106, configured to remove media from cells, wherein each component is in fluid communication with all other components. Cells are cultured into a biomass in the bioreactor 101, while the aerator 105 percolates oxygen and/or carbon dioxide, for a first time period at a first temperature, e.g., a proliferation temperature of 35° C.-40° C. for facilitating cell proliferation. During the first time period, enriched media may be periodically added (also referred to as feeds) through an inlet of the bioreactor 101. After the first time period has elapsed, a portion of cells of the biomass, e.g., 60%, 70%, or 80% or more, are transferred from the bioreactor 101 to the differentiation system. Remaining cells are flooded with fresh media to become a second batch for continuous cultivation. The cells in the differentiation system 102 are cultivated for a second time period at a second temperature, e.g., a heat shock temperature that activates the heat shock promoter. During both the first and second time periods, the cells are continuously agitated to prevent settling, to limit aggregation, and to otherwise maintain the cells in a suspended state, e.g. by an impeller. After the second time period has elapsed, media is removed from the induced cells with centrifuge 106, and the induced cells are harvested for formulation into a comestible food product. Sensors that monitor oxygen, carbon dioxide, temperature, pH, glucose, and so on may also be a part of the bioreactor system. Generally, the factors regulating culture conditions can be controlled by computer or manual input.

In embodiments, the cells are cultured in suspension, suspension aggregates, or suspension microcarriers. Cells cultivated within a bioreactor are often adherent independent, suspension cells.

Selection of cells can be performed as described previously. In embodiments, the selected cells are derived from the same parent cell line, and stably express one or more factors or biomarkers associated with a muscle or fat phenotype during cultivation at a heat shock temperature.

In embodiments, the cells are grown in serum free media in suspension to a cell density of at least about 0.1 mil/mL, 0.2 mil/mL, 0.3 mil/mL, 0.4 mil/mL, 0.5 mil/mL, 1 mil/mL, 1.5 mil/mL, 2 mil/mL, 2.5 mil/mL, 3 mil/mL, 4 mil/mL, 5 mil/mL, 6 mil/mL, 7 mil/mL, 8 mil/mL, 9 mil/mL, 10 mil/mL, 11 mil/mL, 12 mil/mL, 13 mil/mL, 14 mil/mL, 15 mil/mL, 16 mil/mL, 17 mil/mL, 18 mil/mL, 19 mil/mL, 20 mil/mL, 21 mil/mL, 22 mil/mL, 23 mil/mL, 24 mil/mL, 25 mil/mL, 26 mil/mL, 27 mil/mL, 28 mil/mL, 29 mil/mL, 30 mil/mL, 31 mil/mL, 32 mil/mL, 33 mil/mL, 34 mil/mL, 35 mil/mL, 36 mil/mL, 37 mil/mL, 38 mil/mL, 39 mil/mL, 40 mil/mL, 41 mil/mL, 42 mil/mL, 43 mil/mL, 44 mil/mL, 45 mil/mL, 46 mil/mL, 47 mil/mL, 48 mil/mL, 49 mil/mL, 50 mil/mL, 51 mil/mL, 52 mil/mL, 53 mil/mL, 54 mil/mL, 55 mil/mL, 56 mil/mL, 57 mil/mL, 58 mil/mL, 59 mil/mL, 60 mil/mL, 61 mil/mL, 62 mil/mL, 63 mil/mL, 64 mil/mL, 65 mil/mL, 66 mil/mL, 67 mil/mL, 68 mil/mL, 69 mil/mL, or about 70 mil/mL.

In terms of scale, the bioreactors and differentiation systems described herein are configured to hold a suspension culture volume of at least 20,000 L. In embodiments, the vessel can hold a volume of suspension culture that is about 20,000 L, about 25,000 L, about 50,000 L, about 100,000 L, about 200,000 L, about 250,000 L, or about 500,000 L. The culture vessel can hold a volume of suspension culture, for example and without limitation, from about 20,000 L to about 50,000 L, from about 20,000 L to about 100,000 L, from about 20,000 L to about 250,000 L, from about 20,000 L to about 500,000 L, from about 25,000 L to about 50,000 L, from about 25,000 L to about 100,000 L, from about 25,000 L to about 250,000 L, from about 25,000 L to about 500,000 L, from about 50,000 L to about 100,000 L, from about 50,000 L to about 250,000 L, from about 50,000 L to about 500,000 L, from about 100,000 L to about 250,000 L, from about 100,000 L to about 500,000 L, from about 200,000 L to about 250,000 L, from about 200,000 L to about 500,000 L, or from about 250,000 L to about 500,000 L. In some embodiments, the clonally derived cells are cultivated until they reach a packed cell volume (PCV) of about 6%. In some embodiments, the inducible cells are cultivated at a large scale for commercial usage.

In some embodiments, for the proliferative phase (i.e., below heat shock temperature) of non-human animal, non-human mammalian, or bovine cells, the cells are cultured at 37° C. between 20 and 40 days. In embodiments, the cells are cultured at 37° C. for about 20 days to 40 days, for about 20 to 39 days, for about 20 to 38 days, for about 20 to 37 days, for about 20 to 36 days, for about 20 to 35 days, for about 20 to 34 days, for about 20 to 33 days, for about 20 to 32 days, for about 20 to 31 days, for about 20 to 30 days, for about 20 to 29 days, for about 20 to 28 days, for about 20 to 27 days, for about 20 to 26 days, for about 20 to 25 days, for about 20 to 24 days, for about 20 to 23 days, for about 20 to 22 days, for about 20 to 21 days, for about 21 to 40 days, for about 21 to 39 days, for about 21 to 38 days, for about 21 to 37 days, for about 21 to 36 days, for about 21 to 35 days, for about 21 to 34 days, for about 21 to 33 days, for about 21 to 32 days, for about 21 to 31 days, for about 21 to 30 days, for about 21 to 29 days, for about 21 to 28 days, for about 21 to 27 days, for about 21 to 26 days, for about 21 to 25 days, for about 21 to 24 days, for about 21 to 23 days, for about 21 to 22 days, for about 22 days to 40 days, for about 22 to 39 days, for about 22 to 38 days, for about 22 to 37 days, for about 22 to 36 days, for about 22 to 35 days, for about 22 to 34 days, for about 22 to 33 days, for about 22 to 32 days, for about 22 to 31 days, for about 22 to 30 days, for about 22 to 29 days, for about 22 to 28 days, for about 22 to 27 days, for about 22 to 26 days, for about 22 to 25 days, for about 22 to 24 days, for about 22 to 23 days, for about 23 days to 40 days, for about 23 to 39 days, for about 23 to 38 days, for about 23 to 37 days, for about 23 to 36 days, for about 23 to 35 days, for about 23 to 34 days, for about 23 to 33 days, for about 23 to 32 days, for about 23 to 31 days, for about 23 to 30 days, for about 23 to 29 days, for about 23 to 28 days, for about 23 to 27 days, for about 23 to 26 days, for about 23 to 25 days, for about 23 to 24 days, for about 24 days to 40 days, for about 24 to 39 days, for about 24 to 38 days, for about 24 to 37 days, for about 24 to 36 days, for about 24 to 35 days, for about 24 to 34 days, for about 24 to 33 days, for about 24 to 32 days, for about 24 to 31 days, for about 24 to 30 days, for about 24 to 29 days, for about 24 to 28 days, for about 24 to 27 days, for about 24 to 26 days, for about 24 to 25 days, for about 25 days to 40 days, for about 25 to 39 days, for about 25 to 38 days, for about 25 to 37 days, for about 25 to 36 days, for about 25 to 35 days, for about 25 to 34 days, for about 25 to 33 days, for about 25 to 32 days, for about 25 to 31 days, for about 25 to 30 days, for about 25 to 29 days, for about 25 to 28 days, for about 25 to 27 days, for about 25 to 26 days, for about 26 days to 40 days, for about 26 to 39 days, for about 26 to 38 days, for about 26 to 37 days, for about 26 to 36 days, for about 26 to 35 days, for about 26 to 34 days, for about 26 to 33 days, for about 26 to 32 days, for about 26 to 31 days, for about 26 to 30 days, for about 26 to 29 days, for about 26 to 28 days, for about 26 to 27 days, for about 27 days to 40 days, for about 27 to 39 days, for about 27 to 38 days, for about 27 to 37 days, for about 27 to 36 days, for about 27 to 35 days, for about 27 to 34 days, for about 27 to 33 days, for about 27 to 32 days, for about 27 to 31 days, for about 27 to 30 days, for about 27 to 29 days, for about 27 to 28 days, 28 to 40 days, for about 28 to 39 days, for about 28 to 38 days, for about 28 to 37 days, for about 28 to 36 days, for about 28 to 35 days, for about 28 to 34 days, for about 28 to 33 days, for about 28 to 32 days, for about 28 to 31 days, for about 28 to 30 days, for about 28 to 29 days, for about 29 to 40 days, for about 29 to 39 days, for about 29 to 38 days, for about 29 to 37 days, for about 29 to 36 days, for about 29 to 35 days, for about 29 to 34 days, for about 29 to 33 days, for about 29 to 32 days, for about 29 to 31 days, for about 29 to 30 days, for about 30 to 40 days, for about 30 to 39 days, for about 30 to 38 days, for about 30 to 37 days, for about 30 to 36 days, for about 30 to 35 days, for about 30 to 34 days, for about 30 to 33 days, for about 30 to 32 days, for about 30 to 31 days, for about 31 to 40 days, for about 31 to 39 days, for about 31 to 38 days, for about 31 to 37 days, for about 31 to 36 days, for about 31 to 35 days, for about 31 to 34 days, for about 31 to 33 days, for about 31 to 32 days, for about 32 to 40 days for about 32 to 39 days, for about 32 to 38 days, for about 32 to 37 days, for about 32 to 36 days, for about 32 to 35 days, for about 32 to 34 days, for about 32 to 33 days, for about 33 to 40 days for about 33 to 39 days, for about 33 to 38 days, for about 33 to 37 days, for about 33 to 36 days, for about 33 to 35 days, for about 33 to 34 days, for about 34 to 40 days for about 34 to 39 days, for about 34 to 38 days, for about 34 to 37 days, for about 34 to 36 days, for about 34 to 35 days, for about 34 to 40 days for about 34 to 39 days, for about 34 to 38 days, for about 34 to 37 days, for about 34 to 35 days, for about 34 to 36 days, for about 35 to 40 days for about 35 to 39 days, for about 35 to 38 days, for about 35 to 37 days, for about 35 to 36 days, for about 36 to 40 days for about 36 to 39 days, for about 36 to 38 days, for about 36 to 37 days, for about 37 to 40 days for about 37 to 39 days, for about 37 to 38 days, for about 38 to 40 days, for about 38 to 39 days, or for about 39 to 40 days.

In some embodiments, for the proliferative phase (not at heat shock temperatures) of avian or chicken cells, the cells are cultured at 37° C. between 20 and 40 days. In embodiments, the cells are cultured at 37° C. for about 20 days to 40 days, for about 20 to 39 days, for about 20 to 38 days, for about 20 to 37 days, for about 20 to 36 days, for about 20 to 35 days, for about 20 to 34 days, for about 20 to 33 days, for about 20 to 32 days, for about 20 to 31 days, for about 20 to 30 days, for about 20 to 29 days, for about 20 to 28 days, for about 20 to 27 days, for about 20 to 26 days, for about 20 to 25 days, for about 20 to 24 days, for about 20 to 23 days, for about 20 to 22 days, for about 20 to 21 days, for about 21 to 40 days, for about 21 to 39 days, for about 21 to 38 days, for about 21 to 37 days, for about 21 to 36 days, for about 21 to 35 days, for about 21 to 34 days, for about 21 to 33 days, for about 21 to 32 days, for about 21 to 31 days, for about 21 to 30 days, for about 21 to 29 days, for about 21 to 28 days, for about 21 to 27 days, for about 21 to 26 days, for about 21 to 25 days, for about 21 to 24 days, for about 21 to 23 days, for about 21 to 22 days, for about 22 days to 40 days, for about 22 to 39 days, for about 22 to 38 days, for about 22 to 37 days, for about 22 to 36 days, for about 22 to 35 days, for about 22 to 34 days, for about 22 to 33 days, for about 22 to 32 days, for about 22 to 31 days, for about 22 to 30 days, for about 22 to 29 days, for about 22 to 28 days, for about 22 to 27 days, for about 22 to 26 days, for about 22 to 25 days, for about 22 to 24 days, for about 22 to 23 days, for about 23 days to 40 days, for about 23 to 39 days, for about 23 to 38 days, for about 23 to 37 days, for about 23 to 36 days, for about 23 to 35 days, for about 23 to 34 days, for about 23 to 33 days, for about 23 to 32 days, for about 23 to 31 days, for about 23 to 30 days, for about 23 to 29 days, for about 23 to 28 days, for about 23 to 27 days, for about 23 to 26 days, for about 23 to 25 days, for about 23 to 24 days, for about 24 days to 40 days, for about 24 to 39 days, for about 24 to 38 days, for about 24 to 37 days, for about 24 to 36 days, for about 24 to 35 days, for about 24 to 34 days, for about 24 to 33 days, for about 24 to 32 days, for about 24 to 31 days, for about 24 to 30 days, for about 24 to 29 days, for about 24 to 28 days, for about 24 to 27 days, for about 24 to 26 days, for about 24 to 25 days, for about 25 days to 40 days, for about 25 to 39 days, for about 25 to 38 days, for about 25 to 37 days, for about 25 to 36 days, for about 25 to 35 days, for about 25 to 34 days, for about 25 to 33 days, for about 25 to 32 days, for about 25 to 31 days, for about 25 to 30 days, for about 25 to 29 days, for about 25 to 28 days, for about 25 to 27 days, for about 25 to 26 days, for about 26 days to 40 days, for about 26 to 39 days, for about 26 to 38 days, for about 26 to 37 days, for about 26 to 36 days, for about 26 to 35 days, for about 26 to 34 days, for about 26 to 33 days, for about 26 to 32 days, for about 26 to 31 days, for about 26 to 30 days, for about 26 to 29 days, for about 26 to 28 days, for about 26 to 27 days, for about 27 days to 40 days, for about 27 to 39 days, for about 27 to 38 days, for about 27 to 37 days, for about 27 to 36 days, for about 27 to 35 days, for about 27 to 34 days, for about 27 to 33 days, for about 27 to 32 days, for about 27 to 31 days, for about 27 to 30 days, for about 27 to 29 days, for about 27 to 28 days, 28 to 40 days, for about 28 to 39 days, for about 28 to 38 days, for about 28 to 37 days, for about 28 to 36 days, for about 28 to 35 days, for about 28 to 34 days, for about 28 to 33 days, for about 28 to 32 days, for about 28 to 31 days, for about 28 to 30 days, for about 28 to 29 days, for about 29 to 40 days, for about 29 to 39 days, for about 29 to 38 days, for about 29 to 37 days, for about 29 to 36 days, for about 29 to 35 days, for about 29 to 34 days, for about 29 to 33 days, for about 29 to 32 days, for about 29 to 31 days, for about 29 to 30 days, for about 30 to 40 days, for about 30 to 39 days, for about 30 to 38 days, for about 30 to 37 days, for about 30 to 36 days, for about 30 to 35 days, for about 30 to 34 days, for about 30 to 33 days, for about 30 to 32 days, for about 30 to 31 days, for about 31 to 40 days, for about 31 to 39 days, for about 31 to 38 days, for about 31 to 37 days, for about 31 to 36 days, for about 31 to 35 days, for about 31 to 34 days, for about 31 to 33 days, for about 31 to 32 days, for about 32 to 40 days for about 32 to 39 days, for about 32 to 38 days, for about 32 to 37 days, for about 32 to 36 days, for about 32 to 35 days, for about 32 to 34 days, for about 32 to 33 days, for about 33 to 40 days for about 33 to 39 days, for about 33 to 38 days, for about 33 to 37 days, for about 33 to 36 days, for about 33 to 35 days, for about 33 to 34 days, for about 34 to 40 days for about 34 to 39 days, for about 34 to 38 days, for about 34 to 37 days, for about 34 to 36 days, for about 34 to 35 days, for about 34 to 40 days for about 34 to 39 days, for about 34 to 38 days, for about 34 to 37 days, for about 34 to 35 days, for about 34 to 36 days, for about 35 to 40 days for about 35 to 39 days, for about 35 to 38 days, for about 35 to 37 days, for about 35 to 36 days, for about 36 to 40 days for about 36 to 39 days, for about 36 to 38 days, for about 36 to 37 days, for about 37 to 40 days for about 37 to 39 days, for about 37 to 38 days, for about 38 to 40 days, for about 38 to 39 days, or for about 39 to 40 days.

In some embodiments, to induce expression of at least one factor or biomarker associated with fat or muscle phenotype under control of a heat shock promoter in bovine cells, the cells are cultured at a heat shock temperature of at 42° C., or at 43° C., or at 44° C. (e.g., a temperature that activates the heat shock promoter but does not significantly (by 1%, 5%, 10%, 15%, 20%, 25%, 30%, or 33%) impact cell viability) for about 1 hour. In embodiments, the cells are cultured at the heat shock temperature for about 1 hour, for about 1.5 hours, for about 2 hours, for about 2.5 hours, for about 3 hours, for about 3.5 hours, for about 4 hours, for about 4.5 hours, for about 5 hours, for about 5.5 hours, for about 6 hours, for about 6.5 hours, for about 7 hours, for about 7.5 hours, for about 8 hours, for about 8.5 hours, for about 9 hours, for about 9.5 hours, for about 10 hours, for about 10.5 hours, for about 11 hours, for about 11.5 hours, for about 12 hours, for about 12.5 hours, for about 13 hours, for about 13.5 hours, for about 14 hours, for about 14.5 hours, for about 15 hours, for about 15.5 hours, for about 16 hours, for about 16.5 hours, for about 17 hours, for about 17.5 hours, for about 18 hours, for about 18.5 hours, for about 19 hours, for about 19.5 hours, for about 20 hours, for about 20.5 hours, for about 21 hours, for about 21.5 hours, for about 22 hours, for about 22.5 hours, for about 23 hours, for about 23.5 hours, for about 24 hours. In other embodiments, the cells are cultured at a heat shock temperature for about 1 hour to 24 hours daily over the span of 30 days.

In some embodiments, to induce expression of at least one factor or biomarker associated with a fat or muscle phenotype under control of a heat shock promoter in avian cells, the cells are cultured at a heat shock temperature (e.g., a temperature that activates the heat shock promoter but does not but does not significantly (by 1%, 5%, 10%, 15%, 20%, 25%, 30%, or 33%) impact cell viability) of 41° C. or 42° C. or 43° C. for about 1 hour. In embodiments, the cells are cultured at a heat shock temperature for about 1 hour, for about 1.5 hours, for about 2 hours, for about 2.5 hours, for about 3 hours, for about 3.5 hours, for about 4 hours, for about 4.5 hours, for about 5 hours, for about 5.5 hours, for about 6 hours, for about 6.5 hours, for about 7 hours, for about 7.5 hours, for about 8 hours, for about 8.5 hours, for about 9 hours, for about 9.5 hours, for about 10 hours, for about 10.5 hours, for about 11 hours, for about 11.5 hours, for about 12 hours, for about 12.5 hours, for about 13 hours, for about 13.5 hours, for about 14 hours, for about 14.5 hours, for about 15 hours, for about 15.5 hours, for about 16 hours, for about 16.5 hours, for about 17 hours, for about 17.5 hours, for about 18 hours, for about 18.5 hours, for about 19 hours, for about 19.5 hours, for about 20 hours, for about 20.5 hours, for about 21 hours, for about 21.5 hours, for about 22 hours, for about 22.5 hours, for about 23 hours, for about 23.5 hours, for about 24 hours. In other embodiments, the cells are cultured at a heat shock temperature for about 1 hour to 24 hours daily over the span of 30 days.

In some embodiments, a substantial portion of the cells used in the disclosed method exhibit expression of one or more factors or biomarkers associated with a muscle or fat phenotype operably linked to the heat shock promoter. In some embodiments, the substantial portion is at least 51% of the cellular population.

In some embodiments, the population of cells is separated from the components of the culture. Separation can be achieved using filtration, centrifugation, gravity, spray drying, or a combination thereof. In some embodiments, the induced population of cells is harvested from in vitro in culture. Once separated from the components of the culture (e.g., the cell culture medium), the population of cells may comprise a cell pellet. The cell pellet is hydrated and/or washed with a solution to produce the hydrated cell mass. Non-limiting examples of solution include water, saline solution, serum-free cell culture medium, and conditioned (filtered) medium from the culture.

In some embodiments, the bioreactor system contains the remaining cells. In some embodiments, additional media flows into the bioreactor in order to reach a desired volume. In some embodiments, the media is serum free. In some embodiments, the cells left in the bioreactor are still in a proliferative state. In some embodiments, the remaining cells will continue to proliferate in the bioreactor having a capacity of at least 20 kiloliters until a PCV of 6% is reached again. In embodiments, this “draw and fill” process will continuously provide proliferative, clonally derived fibroblasts that can differentiate into adipocytes or myotubes.

### 5.4. Chemostat with Cell Retention Culturing Systems and Methods

Provided herein is a chemostat with cell retention culturing method comprising: seeding an inoculum of cells comprising one or more factors or biomarkers associated with a muscle or fat phenotype operably linked to a heat shock promoter; growing the cells for at least 2 days without cell recovery; and partially recovering a fraction of the cells at a steady rate, wherein the partially recovery includes inline heating to 43° C. (or a temperature that activates the heat shock promoter but does not significantly impact cell viability), inline aeration, and a transit time sufficient to induce a substantial portion of the cells. In embodiments, the heat shock promoter is modified to exhibit reduced activation by heavy metals or ultraviolet light as compared to a native heat shock promoter. In embodiments, the heat shock promoter is heterologous to the species of the cells. In other embodiments, wherein the heat shock promoter is homologous to the species of the cells and comprises one or more portions of the heat shock promoter having heat-inducible promoter activity. In some embodiments, the heat shock promoter is a HSP1A1 promoter or HSF-1 promoter. In additional embodiments, the heat shock promoter comprises the nucleotide sequence of SEQ ID NO: 29. In embodiments, the cells used herein are non-human mammalian and non-human animal cells, including but not limited to cells from fish, shellfish, cattle, pigs, sheep, goats, deer, rabbits, and poultry.

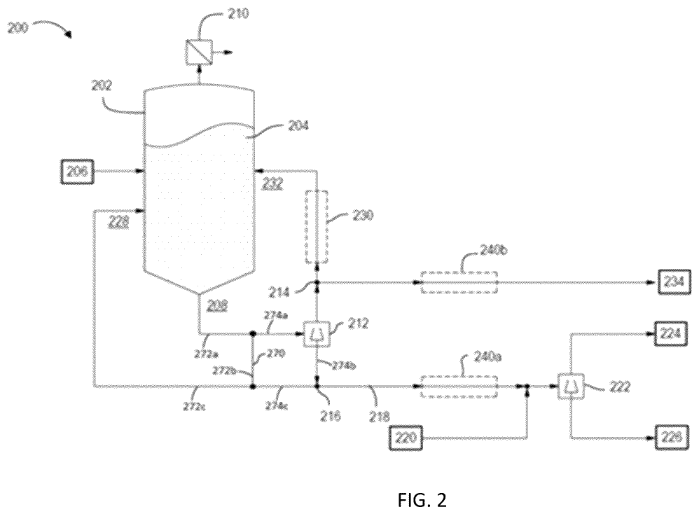

FIG. 2 illustrates a suspension bioreactor system 200 comprised of various components configured for continuous cultivation and recovery of cells within a sterile environment.

As shown in FIG. 2, for instance, the suspension bioreactor system 200 comprises a bioreactor vessel 202 configured to cultivate a suspension culture 204 comprising non-human animal cells, including non-human animal, non-human mammalian, avian, chicken, or bovine cells (e.g., for use in the production of a comestible cell-based food product). In some implementations, for example, the bioreactor vessel 202 comprises a suspension bioreactor with a 10,000 liter capacity. In other implementations, the bioreactor vessel 202 comprises a capacity less or more than 10,000 liters, such as a capacity between 500 liters, 1000, liters, 2000 liters, 5000 liters, 15,000 liters, 20,000 liters, 50,000 liters, 75,000 liters, 100,000 liters, 250,000 liters, etc. As illustrated, the bioreactor vessel 202 is loaded with non-human animal cells (mammalian, avian, chicken, or bovine, in embodiments) and a cell culture media is delivered to the bioreactor vessel 202 (e.g., from a cell culture media stream 206) to cultivate the non-human animal cells therein. As mentioned above, in one or more implementations, cells are initially cultivated within the bioreactor vessel 202 for a first period of time (e.g., until a particular cell density within the suspension culture 204 is achieved such as 5% PCV, 10% PCV, 15% PCV) in preparation for continuous cultivation and partial recovery of cells from the bioreactor vessel 202 during a second period of time.

As further illustrated in FIG. 2, the suspension bioreactor system 200 in cell culture media stream 206 configured to introduce cell culture media (e.g., fresh nutritive media) into the bioreactor vessel 202 to replenish any spent media removed from the bioreactor vessel 202. During initial cultivation, for instance, spent media is removed from the bioreactor vessel 202 while retaining cells within the suspension vessel 202 for continued cultivation via nutrients provided by the cell culture media stream 206. In one or more implementations, cell culture media is flowed into the bioreactor vessel 202 at a gradually increasing flow rate during initial cultivation until a steady state cultivation flow rate, which is maintained during continuous cultivation and partial recovery of cultivated cells. In one or more implementations, for example, the steady state cultivation flow rate comprises about 5 vessel volumes per day (VVD). In other implementations, the steady state cultivation flow rate comprises between 1 VVD and 20 VVD (e.g., 2 VVD, 7 VVD, 10 VVD, 15 VVD, etc.), depending at least in part on the growth rate of cells within the suspension bioreactor.

In certain implementations, a measure of the suspension culture 204, including cultivated cells, is drawn from the bioreactor vessel 202 via a suspension culture line 208 for partial recovery and partial cell retention, as suspension culture within the bioreactor vessel 202 is continuously cultivated (e.g., for the aforementioned second period of time). In some implementations, for example, the suspension culture line 208 draws a stream of suspension culture at a rate of about 14 VVD for partial cell recovery and partial cell retention. In other implementations, the suspension culture line 208 draws a stream of suspension culture at an extraction flow rate between 5 VVD and 20 VVD, depending at least in part on cell growth rates within the bioreactor vessel, cell retention rates, and/or cell recovery rates.

As also shown, the suspension bioreactor system 200 includes a sterile cell retention device 212 configured to separate the measure of the suspension culture 204 from the suspension culture line 208 into a concentrated cell culture (e.g., a heavy phase of 3X cell density compared to the provided suspension culture 204) and a culture broth with cells removed (e.g., a light phase with a negligible or relatively small density of cultivated cells). In some implementations, for example, the sterile cell retention device 212 separates the suspension cell culture stream into one part concentrated cell culture and two parts culture broth with cells removed. The sterile cell retention device 212 can include, for example, a continuous flow centrifuge configured to separate received materials within a sterile environment (e.g., a centrifuge comprising sealed components and/or other features for maintaining sterility within a closed system). Alternative examples of sterile cell retention devices include, but are not limited to, apparatuses configured for sterile filtration, gravity sedimentation, magnetic separation, and so forth.

As illustrated, the suspension bioreactor system 200 further comprises a cell recovery line 218 for recovering cultivated cells from at least a portion of the measure of the suspension culture 204 drawn from the bioreactor vessel 202. In particular, the cell recovery line 218 is configured to receive a first portion of the concentrated cell culture provided by the sterile cell retention device 212 for recovery from the suspension bioreactor system 200. In some embodiments, the cell recovery line 218 raises a temperature of the cell culture therein to about 43° C. (or a temperature that activates the heat shock promoter but does not significantly reduce cell viability) and provides a transit time sufficient to induce expression of one or more factors or biomarkers associated with a muscle or fat phenotype under the control of a heat shock promoter in a substantial majority of the cells of the cell culture via heat shock induced expression. In alternative embodiments, such heat shocked induced expression is performed in a tank. As shown, for example, the suspension bioreactor system 200 also includes a wash buffer line 220 configured to deliver wash buffer to dilute and/or wash the portion of the concentrated cell culture received via the cell recovery line 218 prior to secondary processing via a cell concentration device 222. In some implementations, for example, the wash buffer line 220 introduces one part wash buffer to one part concentrated cell culture received from the sterile cell retention device 212. To recover cultivated cells from the concentrated cell culture, the cell concentration device 222 separates the concentrated cell culture received via the cell recovery line 218 into spent media 224 (e.g., wash buffer and/or cell culture media that is at least partially depleted of nutrients) and recovered cells 226 (e.g., a heavy phase wet mass comprising further concentrated cell culture). In some embodiments, for example, the cell concentration device 222 further concentrates the cell culture by a factor between 3× and 5× to produce a mass of recovered cells 226 comprising about 70% cells per volume (e.g., packed cell volume).

Furthermore, as shown in FIG. 2, the suspension bioreactor system 200 comprises a cell retention line 228 configured to return at least a portion of the concentrated cell culture output by the sterile cell retention device 212 to the bioreactor vessel 202 for continued cultivation. In some implementations, for example, a first portion of the concentrated cell culture is continuously recovered (e.g., removed from the suspension bioreactor system 200) while a second portion of the concentrated cell culture is returned Docu to the bioreactor vessel 202 in order to maintain a particular or desired cell density in the suspension culture 204 within the bioreactor vessel 202 during cultivation. Specifically, a valve or stream divider 216 can be controlled to split the concentrated cell culture to send a first portion to cell recovery via the cell recovery line 218 and a second portion back to the bioreactor vessel 202 via the cell retention line 228. In some embodiments, the cell retention line 228 is in fluid communication with a reservoir comprising cell retentate conditioning materials that may be introduced (e.g., injected) into the cell retention line 228, where the conditioning materials refresh or otherwise revitalize the cell retentate before returning to the bioreactor.

In one or more implementations, the cell retention line 228 enables the maintenance of a higher cell density by mitigating waste component build up, e.g., ammonia and lactate, mitigating spent media accumulation, and helping ensure a constant supply of fresh nutrients, e.g., growth factors and amino acids that have short half-lives. One will appreciate that this would not occur in a simple chemostat style reactor lacking a return of concentrated cells. In some implementations, for example, the suspension bioreactor system 200 recovers between 2.5% to 50% of the concentrated cell culture via the cell recovery line 218, while returning 50% to 97.5% of the concentrated cell culture back into the bioreactor vessel 202. Furthermore, in some implementations, the suspension bioreactor system 200 recovers between 5% to 25% of the concentrated cell culture via the cell recovery line 218, while returning 75% to 95% of the concentrated cell culture back into the bioreactor vessel 202. Still further, in some implementations, the suspension bioreactor system 200 recovers between 5% to 15% of the concentrated cell culture via the cell recovery line 218, while returning 85% to 95% of the concentrated cell culture back into the bioreactor vessel 202. In some implementations, the suspension bioreactor system 200 recovers between 5% to 10% of the concentrated cell culture via the cell recovery line 218, while returning 90% to 95% of the concentrated cell culture back into the bioreactor vessel 202.

In some implementations, it is advantageous to position the cell retention device 212 a particular distance from the bioreactor vessel 202 (e.g., separated by a relatively large distance), to allow for the cell retention device 212, or one or more cell retention devices, to be used with multiple reactors, and to reduce certain design constraints (e.g., allowing flexibility in floor plans and equipment layouts). However, a retention time of the cells passing through the cell retention device 212 and back to the bioreactor vessel 202 should be a short enough time length such that the cells do not succumb to hypoxia, pH shifts, excessive CO2 accumulation, temperature fluctuation, or experience other related negative effects, while separated from the bioreactor vessel 202, which is continually oxygenated, to ensure the cells' continued ability to proliferate after returning to the bioreactor vessel 202.

As also shown in FIG. 2, the suspension bioreactor system 200 includes a culture broth retention line 232 configured to return at least a portion of the culture broth with cells removed to the bioreactor vessel 202 for further cultivation of cells therein. As illustrated, in certain implementations the culture broth retention line 232 includes a media management module 230 configured to process (e.g., condition) the culture broth for reintroduction into the bioreactor vessel 202. To illustrate, media processing performed by the media management module 230 can include, but is not limited to, gas stripping, small bubble sparge, air lifting, media conditioning, or introduction of shear protectants (e.g., using pluronics). Further, in one or more implementations, at least a portion of the culture broth with cells removed, comprising spent media 234, is discarded or otherwise removed from the suspension bioreactor system 200. In some implementations, for example, about one part of the culture broth with cells removed is removed as spent media 234, while one part of the culture broth is reintroduced into the bioreactor vessel 202. Specifically, a valve or stream divider 214 can be controlled to split the culture broth with cells removed to send a first portion to be removed as spent media 234 and a second portion back to the bioreactor vessel 202 via the culture broth retention line 232.

As also shown in FIG. 2, the suspension bioreactor system 200 includes various elements for maintaining a sterile environment during the foregoing procedures by reducing and/or preventing contamination from entering the suspension bioreactor system 200. As illustrated, for instance, the bioreactor vessel 202 comprises a sterile vent filter 210 configured to allow excess vapors and/or gasses to exit the bioreactor vessel 202 while restricting contamination from elements outside of the bioreactor vessel 202. In one or more implementations, as illustrated, the suspension bioreactor system 200 includes one or more sterile output modules 240a, 240b, each configured to prevent backflow contamination as materials are extracted from the bioreactor system 200. Specifically, the sterile output module 240a is configured to prevent backflow contamination from entering the sterile environment of the suspension bioreactor system 200 during continual drawing of cells from the concentrated cell culture during cell recovery (e.g., by preventing backflow contamination between the cell concentration device 222 and at least one of the cell retention stream or the sterile cell retention device). Similarly, the sterile output module 240b is configured to prevent backflow contamination from entering the suspension bioreactor system 200 as the spent media 234 is removed from the sterile environment of the suspension bioreactor system 200. In one or more implementations, the sterile output modules can be implemented to provide boundaries between sterile and non-sterile components of the suspension bioreactor system.

In some embodiments, the cells are cultured in serum free media. Serum free media refers to media that is substantially or virtually free of serum. In some embodiments, the cell culture media is hormone free. In some embodiments, the cell culture media is heavy metal free. In some embodiments, the cell culture media is antibiotic free. In other embodiments, the cell culture media is animal-component free. In embodiments, the cells, including non-human animal, non-human mammalian, avian, bovine, or chicken cells, are grown in serum free media in suspension to a cell density of at least 0.1 mil/mL, 0.2 mil/mL, 0.3 mil/mL, 0.4 mil/mL, 0.5 mil/mL, 1 mil/mL, 1.5 mil/mL, 2 mil/mL, 2.5 mil/mL, 3 mil/mL, 4 mil/mL, 5 mil/mL, 6 mil/mL, 7 mil/mL, 8 mil/mL, 9 mil/mL, 10 mil/mL, 11 mil/mL, 12 mil/mL, 13 mil/mL, 14 mil/mL, 15 mil/mL, 16 mil/mL, 17 mil/mL, 18 mil/mL, 19 mil/mL, 20 mil/mL, 21 mil/mL, 22 mil/mL, 23 mil/mL, 24 mil/mL, 25 mil/mL, 26 mil/mL, 27 mil/mL, 28 mil/mL, 29 mil/mL, 30 mil/mL, 31 mil/mL, 32 mil/mL, 33 mil/mL, 34 mil/mL, 35 mil/mL, 36 mil/mL, 37 mil/mL, 38 mil/mL, 39 mil/mL, 40 mil/mL, 41 mil/mL, 42 mil/mL, 43 mil/mL, 44 mil/mL, 45 mil/mL, 46 mil/mL, 47 mil/mL, 48 mil/mL, 49 mil/mL, 50 mil/mL, 51 mil/mL, 52 mil/mL, 53 mil/mL, 54 mil/mL, 55 mil/mL, 56 mil/mL, 57 mil/mL, 58 mil/mL, 59 mil/mL, 60 mil/mL, 61 mil/mL, 62 mil/mL, 63 mil/mL, 64 mil/mL, 65 mil/mL, 66 mil/mL, 67 mil/mL, 68 mil/mL, 69 mil/mL, or at least about 70 mil/mL.

In terms of scale, the bioreactors described herein are configured to hold a suspension culture volume of at least 20,000 L. In embodiments, the vessel can hold a volume of suspension culture that is about 20,000 L, about 25,000 L, about 50,000 L, about 100,000 L, about 200,000 L, about 250,000 L, or about 500,000 L. The culture vessel can hold a volume of suspension culture, for example and without limitation, from about 20,000 L to about 50,000 L, from about 20,000 L to about 100,000 L, from about 20,000 L to about 250,000 L, from about 20,000 L to about 500,000 L, from about 25,000 L to about 50,000 L, from about 25,000 L to about 100,000 L, from about 25,000 L to about 250,000 L, from about 25,000 L to about 500,000 L, from about 50,000 L to about 100,000 L, from about 50,000 L to about 250,000 L, from about 50,000 L to about 500,000 L, from about 100,000 L to about 250,000 L, from about 100,000 L to about 500,000 L, from about 200,000 L to about 250,000 L, from about 200,000 L to about 500,000 L, or from about 250,000 L to about 500,000 L.

In embodiments, the first period of time to allow for proliferation and cultivation is 1 to 14 days. In some embodiments, the first period of time is for 1 to 14 days, 1 to 13 days, 1 to 12 days, 1 to 11 days, 1 to 10 days, 1 to 9 days, 1 to 8 days, 1 to 7 days, 1 to 6 days, 1 to 5 days, 1 to 4 days, 1 to 3 days, 1 to 2 days, 2 to 14 days, 2 to 13 days, 2 to 12 days, 2 to 11 days, 2 to 10 days, 2 to 9 days, 2 to 8 days, 2 to 7 days, 2 to 6 days, 2 to 5 days, 2 to 4 days, 2 to 3 days, 3 to 14 days, 3 to 13 days, 3 to 12 days, 3 to 11 days, 3 to 10 days, 3 to 9 days, 3 to 8 days, 3 to 7 days, 3 to 6 days, 3 to 5 days, 3 to 4 days, 4 to 14 days, 4 to 13 days, 4 to 12 days, 4 to 11 days, 4 to 10 days, 4 to 9 days, 4 to 8 days, 4 to 7 days, 4 to 6 days, 4 to 5 days, 5 to 14 days, 5 to 13 days, 5 to 12 days, 5 to 11 days, 5 to 10 days, 5 to 9 days, 5 to 8 days, 5 to 7 days, 5 to 6 days, 6 to 14 days, 6 to 13 days, 6 to 12 days, 6 to 11 days, 6 to 10 days, 6 to 9 days, 6 to 8 days, 6 to 7 days, 7 to 14 days, 7 to 13 days, 7 to 12 days, 7 to 11 days, 7 to 10 days, 7 to 9 days, 7 to 8 days, 8 to 14 days, 8 to 13 days, 8 to 12 days, 8 to 11 days, 8 to 10 days, 8 to 9 days, 9 to 14 days, 9 to 13 days, 9 to 12 days, 9 to 11 days, 9 to 10 days, 10 to 14 days, 10 to 13 days, 10 to 12 days, 10 to 11 days, 11 to 14 days, 11 to 13 days, 11 to 12 days, 12 to 14 days, 12 to 13 days, or 13 to 14 days. In other embodiments, the first period of time is 1 to 30 days.

In embodiments, the proliferation temperature is between or at 35° C. and 40° C. In further embodiments, the proliferation temperature is between 35° C. and 40° C., between 35° C. and 39° C., between 35° C. and 38° C., between 35° C. and 37° C., or between 35° C. and 36° C. In even further embodiments, the proliferation temperature is 35° C., 36° C., 37° C., 38° C., 39° C., or 40° C. The proliferation temperature is optimized for culture of the particular cell line (e.g., to achieve a target doubling time and/or target cell density) without inducing heat shock expression. In embodiments, the heat shock temperature is between or at 37° C. and 45° C. In further embodiments, the heat shock temperature is between 37° C. and 45° C., between 37° C. and 44° C., between 37° C. and 43° C., between 37° C. and 42° C., between 37° C. and 41° C., between 37° C. and 40° C., between 37° C. and 39° C., or between 37° C. and 38° C. In even further embodiments, the heat shock temperature is 37° C., 38° C., 39° C., 40° C., 41° C., 42° C., 43° C., 44° C., and 45° C.

In embodiments, the proliferation temperature range and heat shock temperature range overlap, and the factor or biomarker associated with a fat or muscle phenotype is expressed at a heat shock temperature 3° C. to 7° C. higher than the proliferation temperature. In embodiments, the heat shock temperature is at least 3° C. higher than the proliferation temperature.

In other embodiments, the second time period is the time it takes to activate the heat shock promoter. In embodiments, the second time period is 1 to 6 hours. In some embodiments, the second time period is 1 to 6 hours, 1 to 5 hours, 1 to 4 hours, 1 to 3 hours, 1 to 2 hours, 2 to 6 hours, 2 to 5 hours, 2 to 4 hours, 2 to 3 hours, 3 to 6 hours, 3 to 5 hours, 3 to 4 hours, 4 to 6 hours, 4 to 5 hours, or 5 to 6 hours. In other embodiments, the cells are cultured at a heat shock temperature for a second time period for 1 to 24 hours. In embodiments, the cells are cultured at a heat shock temperature for about 1 hour, for about 1.5 hours, for about 2 hours, for about 2.5 hours, for about 3 hours, for about 3.5 hours, for about 4 hours, for about 4.5 hours, for about 5 hours, for about 5.5 hours, for about 6 hours, for about 6.5 hours, for about 7 hours, for about 7.5 hours, for about 8 hours, for about 8.5 hours, for about 9 hours, for about 9.5 hours, for about 10 hours, for about 10.5 hours, for about 11 hours, for about 11.5 hours, for about 12 hours, for about 12.5 hours, for about 13 hours, for about 13.5 hours, for about 14 hours, for about 14.5 hours, for about 15 hours, for about 15.5 hours, for about 16 hours, for about 16.5 hours, for about 17 hours, for about 17.5 hours, for about 18 hours, for about 18.5 hours, for about 19 hours, for about 19.5 hours, for about 20 hours, for about 20.5 hours, for about 21 hours, for about 21.5 hours, for about 22 hours, for about 22.5 hours, for about 23 hours, for about 23.5 hours, for about 24 hours.

In embodiments, the expression of one or more factors or biomarkers associated with a fat or muscle phenotype is higher in heat shocked cells than non-transfected cells or cells cultured at a standard temperature for a time. In further embodiments, the expression of one or more biomarkers associated with a fat or muscle phenotype is substantially higher in heat shocked cells than non-transfected cells or cells cultured at a standard temperature for a time. In some embodiments, the expression of one or more biomarkers associated with a fat or muscle phenotype is at 0.5 to 30 fold higher in heat shocked cells than non-transfected cells or cells cultured at a standard temperature for a time. In other embodiments, the expression of the expression of one or more biomarkers associated with a fat or muscle phenotype is 0.5 fold, 1 fold, 2 fold, 3 fold, 4 fold, 5 fold, 6 fold, 7 fold, 8 fold, 9 fold, 10 fold, 11 fold, 12 fold, 13 fold, 14 fold, 15 fold, 16 fold, 17 fold, 18 fold, 19 fold, 20 fold, 21 fold, 22 fold, 23 fold, 24 fold, 25 fold, 26 fold, 27 fold, 28 fold, 29 fold or 30 fold higher in heat shocked cells than non-transfected cells or cells cultured at a standard temperature for a time.

### 6. EXAMPLES

### 6.1. Example 1: Inducible Heat Shock Promoter

This study had two main objectives: (1) to investigate if gene expression can be selectively induced using the Bovine Heat Shock Protein (HSP70) Promoter and (2) determine if overexpression of genes of interest (GOIs) relate to relevant phenotypes without compromising cell growth.

In order to assess the functionality of the heat shock promoter, stable cell lines were generated with a vector as disclosed in U.S. Pat. Application Ser. No. 63/580,265, filed Sep. 1, 2023, which is incorporated herein by reference. The objectives here were to determine if the heat shock promoter increased reporter gene expression, and what temperature and exposure time were required to do so. Briefly, bovine fibroblast 4CTCC cells cultured in 6 well plates in serum containing media at 37° C. were transfected with 4 μg of DNA (helper and donor plasmids) at a 1:4 helper:donor ratio using lipofection. After 48 hours, cells were sorted for mRuby and GFP (transient non-integrating markers on helper and donor plasmids, respectively). Plasmid vectors were constructed containing the following elements: a bacterial backbone with Ampicillin resistance and a high copy pUC origin of replication, a transient non-integrating copGFP-T2A-Puro coding sequence driven by a hEF1a promoter and terminated by a SV40 poly A signal and a WPRE element, piggybac ITR regions specifying the region to be inserted into the genome, HS4 insulator sequences adjacent to the ITRs to prevent transgene silencing, and a region designated for a promoter and transgene to be cloned into, and a Bovine Growth Hormone poly A signal downstream of the Promoter-CDS designated region. The HSPA1A promoter (SEQ ID NO: 29) genomic DNA sequence was extracted from the most recent RefSeq bovine reference genome ARS-UCD2.0 (NCBI RefSeq Assembly: GCF_002263795.3, https://www.ncbi.nlm.nih.gov/datasets/genome/GCF_002263795.3) and synthesized to drive a transgene construct consisting of the following elements linked in order: mRuby-mODC1 destabilizer domain-T2A-Nanoluciferase-PEST destabilizer domain. This synthesized DNA was then cloned into the vector described above using homology cloning. Final plasmid constructs were sequence verified using NGS and scaled up using standard plasmid prep methods.

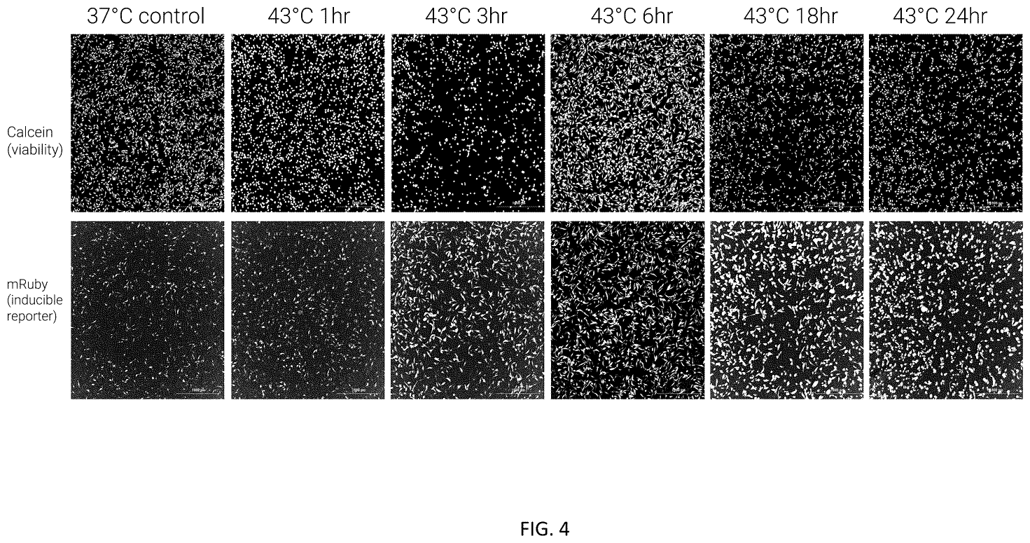

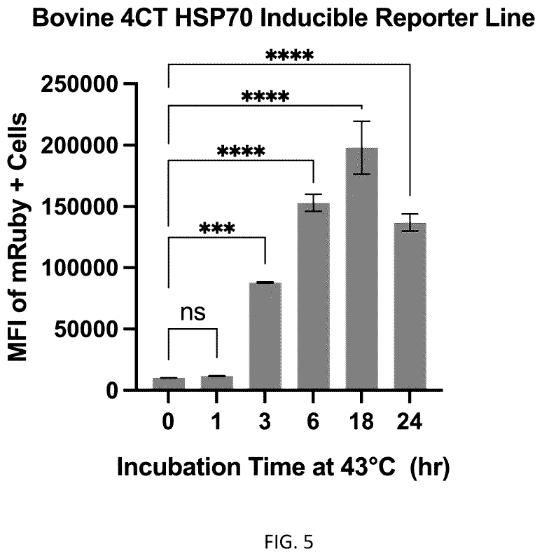

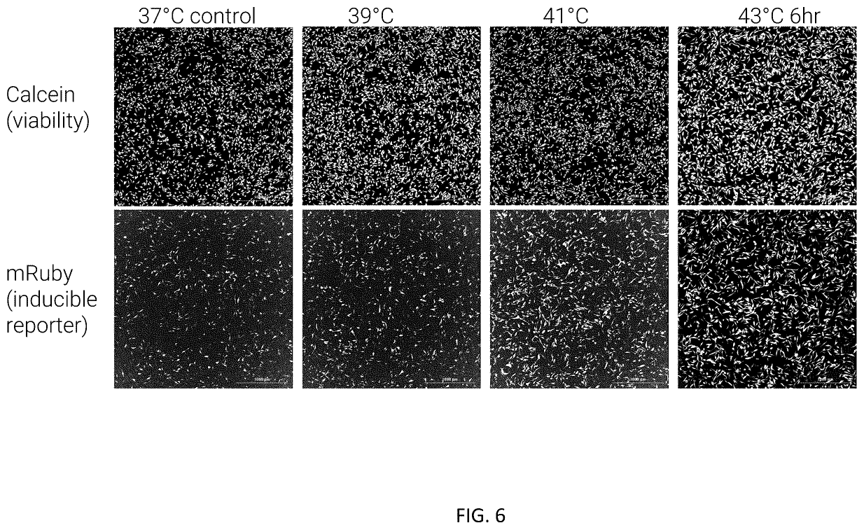

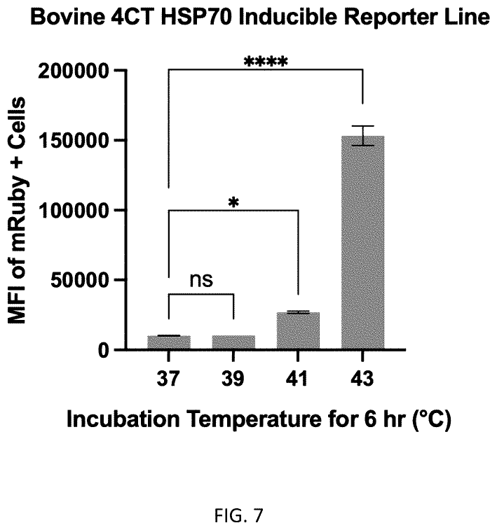

After sorting, cells were expanded for 2 weeks and then heat shock induction was tested in 6 well plates by exposing cells to 43° C. for 0, 1, 3, 6, 18, and 24 hours. In addition, the following temperatures were tested with 6 hours of exposure: 37° C., 39° C., 41° C., and 43°. After the heat shock treatment, cells were immediately live-cell stained in PBS with 2 μM Calcein AM for viability and 1 drop of Hoechst 33342 nuclear counterstain and imaged for fluorescence. n=2 per condition. After imaging, cells were trypsinized and analyzed using flow cytometry. To process flow cytometry data, single cells were gated, Hoechst positive cells (containing nuclei) were gated, and the mRuby gate was set to include 0.1% of naive cells. Median fluorescence intensity (MFI) was computed from cells within the mRuby gate for each condition. Heat shocked cells were viable by Calcein AM staining and exhibited induced reporter gene expression. (FIG. 3). Flow cytometry data indicates that heat shock induced cells exhibited a statistically significant 13 fold increase in fluorescence intensity after incubation at 43° C. for 18 hours compared to the untreated control. The 3 hour and 6 hour 43° C. heat shock conditions also exhibited increases in MFI, roughly proportional to the exposure time. MFI was reduced at 24 hours of 43° C. exposure time; morphological changes were also observed, likely due to issues with cell viability after the extended exposure to 43° C. heat shock (FIGS. 4 and 5). Next, the cells were incubated at 37° C., 39° C., 41° C. and 43° C. for 6 hours. Cells incubated at 39° C. did not produce a significant increase in MFI compared to 41° C. however, the MFI at 41° C. was 5.7 fold less than the MFI measured at 43° C. (FIG. 67).

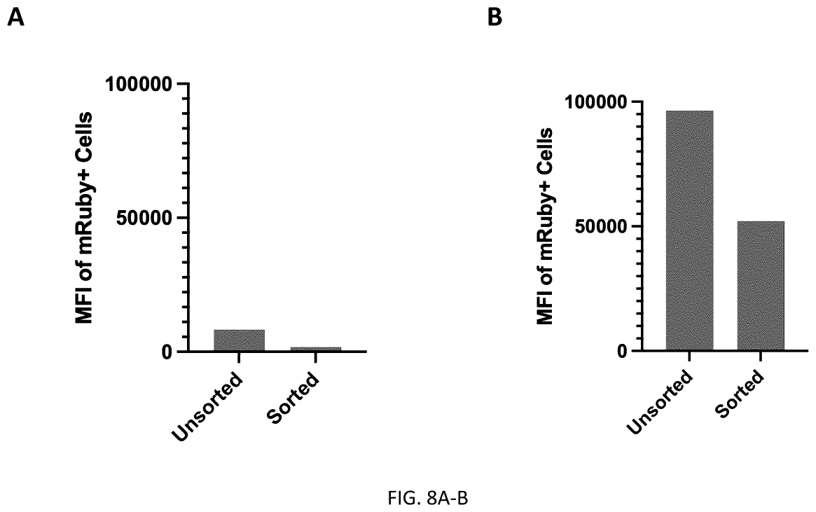

In the non-heat shocked controls for these experiments, a low level of fluorescence was detectable, indicating some level of “leaky expression” (i.e., expression in the absence of the inducer agent). Basal leaky expression is a recognized phenomenon for natural HSP systems. Because an objective of this system is minimal to no expression of the gene of interest during the proliferation phase in un-induced cells, methods for reducing or eliminating leaky expression were tested. To this end, the polyclonal population of stably transfected cells were selected for cells that did not exhibit significant fluorescence in the absence of heat shock, and the populations of cells that exhibited the significant detectable leaky expression were removed. After allowing cells to recover, they were again exposed to heat shock at 43° C. for 0 and 6 hours and analyzed by flow cytometry. The uninduced sorted population of cells showed significantly reduced leaky expression compared to the unsorted parent population; however, the heat shock induced reporter expression was also reduced in the sorted population, leaving more opportunity for optimization. The induced expression was reduced by 1.8 fold compared to the parent population while the leaky expression was reduced by 4.5 fold (FIGS. 8A-B). These results indicate that there are populations of cells in the parental stable cell line that express the reporter gene at different basal levels, in un-induced and induced states.

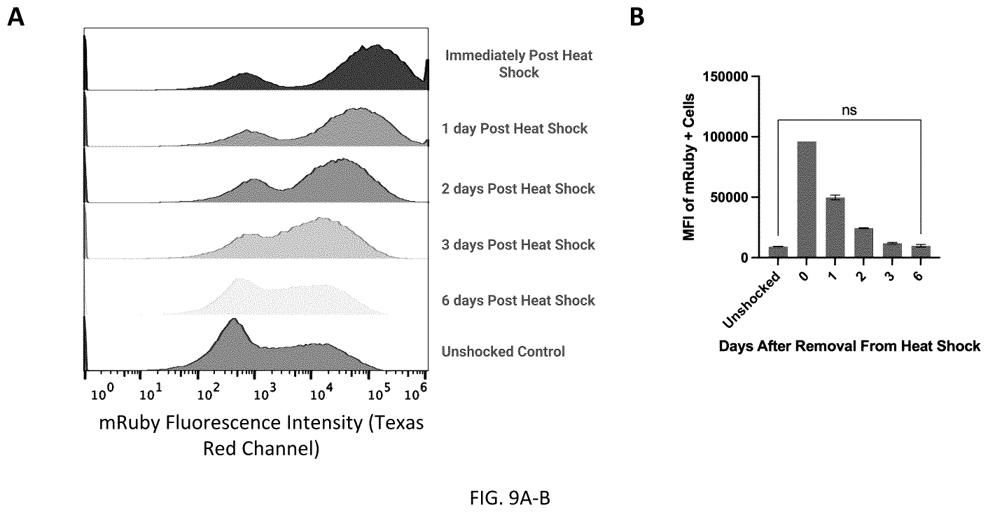

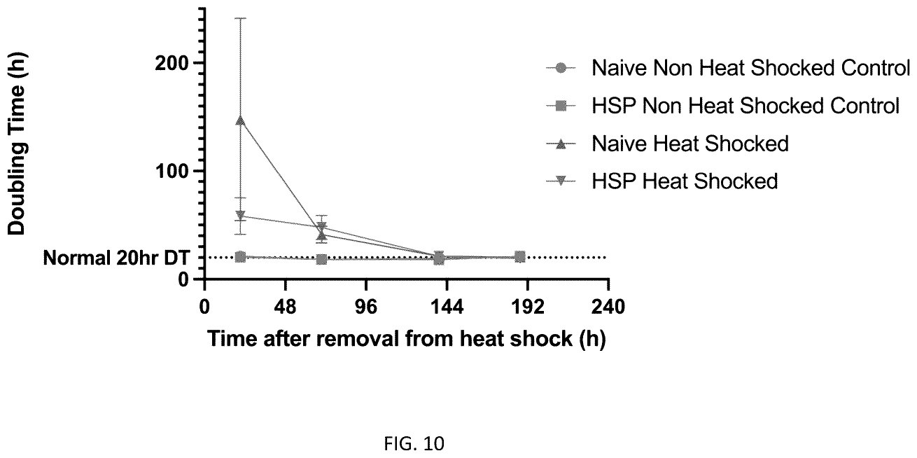

Heat shock induced cellular damage was assessed by examining gene expression and viability after heat shock treatment. Cells were heat shocked for 6 hours at 43° C., then returned to 37° C. Cells were evaluated for fluorescence by flow cytometry at the following time points: immediately post heat shock, and 1, 2, 3, and 6 days post heat shock. Heat shock induced mRuby expression decreased over time after heat shock, returning to background levels after 6 days with roughly a 24 hour half-life of fluorescence intensity. Cells remained viable and continued to proliferate after heat shock, with a slight lag in growth (FIGS. 9A-B). These data indicate that the cells remain in a transcriptionally activated state for some time after heat shock. After 6 days, the MFI returned to background levels and was not statistically significantly different from the un-induced control. To further assess observed morphological changes and slower growth rates after heat shock, Bovine 4CTCC naive and heat shock inducible reporter line cells were seeded in 6 well plates at 200,000 cells per well (n=2), and then immediately heat shocked at 43° C. for 6 hours. The control was kept at 37° C. with no heat shock. After the heat shock, the plates were incubated at 37° C. After 24 hours, cells from both experimental and control plates were trypsinized and cells were counted before seeding new 6 well plates at about 300,000 cells per well. To ensure all cells were accounted for, post-trypsinization wells were washed with media, and the wash media was added to the trypsinized cells before counting. Doubling time was tracked for four passages post-heat shock. Immediately post-heat shock, the doubling time sharply increased, indicating a decline in proliferation rates, for both the naive and HSP reporter line conditions compared to the non-heat shocked controls. Over two passages (5 days), the doubling time of the heat shocked conditions returned to the normal −20 hr baseline doubling time, which is indistinguishable from the non-heat shocked controls, as shown in FIG. 10. These data show that cells remain viable and proliferative after heat shock treatment without long term impacts on doubling time.

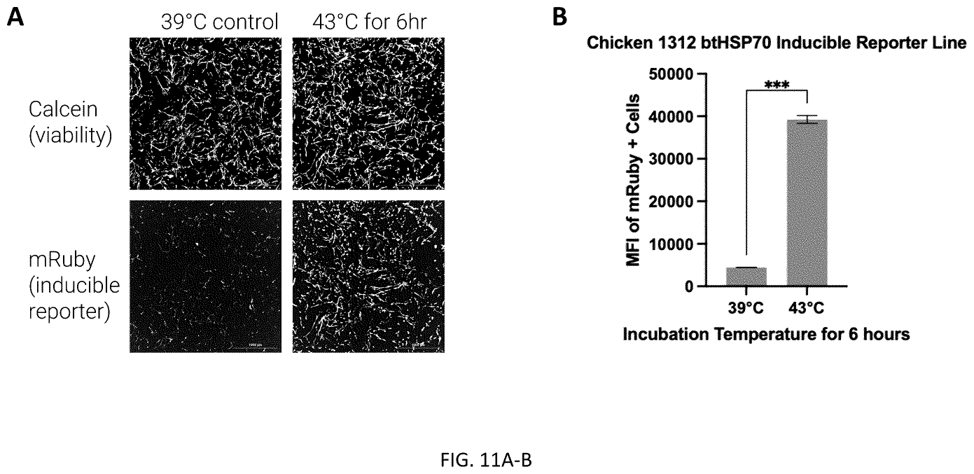

To test these methods in a chicken cell line, identical methods as described above were followed. In summary, a bovine heat shock promoter was integrated and drove expression in a chicken 1312 fibroblast cell line. The cell line was characterized by testing exposure to 43° C. for 6 hours and analyzing by Calcein AM viability staining with fluorescence imaging and flow cytometry. Results indicate that the bovine heat shock promoter also functions as an inducible system in chicken cells, although with a less strong increase in MFI than observed in bovine cells (FIGS. 11A-B). The chicken cells remained viable and exhibited an 8.9 fold increase in reporter expression after heat shock at 43° C. for 6 hours compared to the un-shocked control.

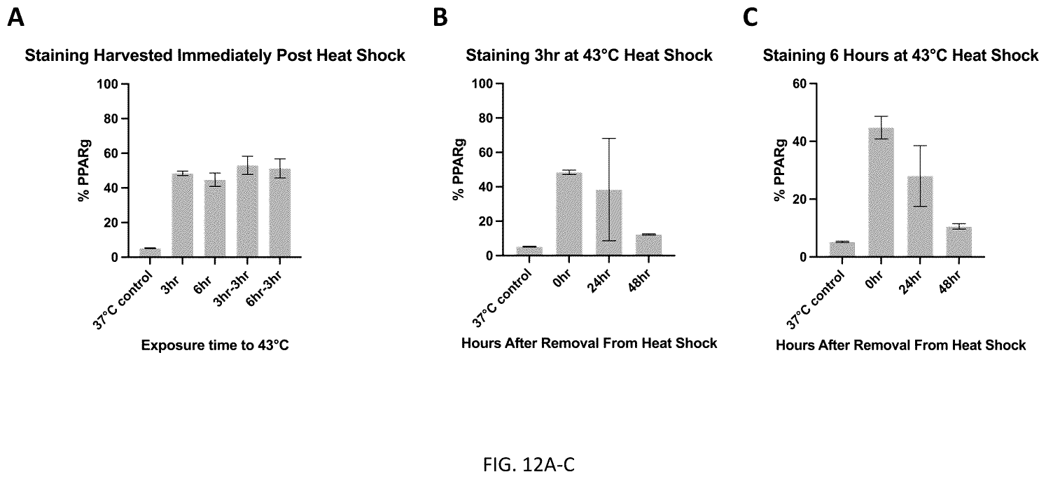

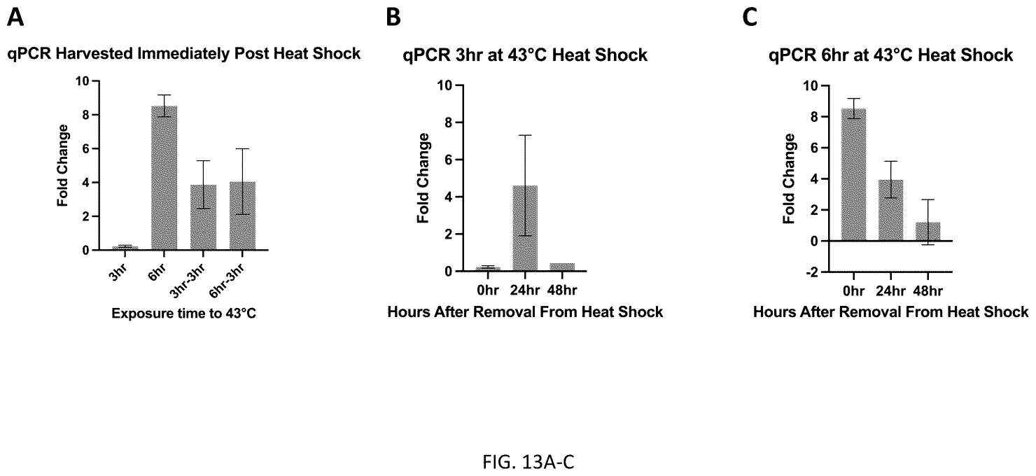

After identifying and characterizing the heat shock promoter with a fluorescent reporter as a proof of concept, heat induction expression was tested for the gene PPARG expression of which is associated with a fat phenotype. New plasmid vectors swapping mRuby for PPARG were constructed using Gibson cloning with the inducible heat shock promoter. Heat inducible PPARG cell lines were generated in 13i bovine cells using lipofection and a 1:4 mass ratio of helper:donor plasmids. These cell lines were sorted using the same dual fluorescence gating FACS strategy as described above and then expanded. Cells were heat shocked at 43° C. for 0 hours, 3 hours, 2×3 hour exposures separated by 24 hours at the normal incubation temperature, 6 hours, and 6 hours followed 24 hours later by 3 hours exposure. Cells for RNA extraction were collected immediately post-heat shock, 24 hours post-heat shock, and 48 hours post-heat shock. Cells were also fixed for immunofluorescence staining at each of those time points. RNA was extracted and reverse transcribed into cDNA before performing qPCR for PPARG to determine mRNA levels. qPCR fold changes were calculated using the Delta Delta CT (ΔΔCT) analysis, using GAPDH as the housekeeping gene and comparing experimental samples to the non-heat shocked control. Fixed cells were stained for PPARG and imaged with a fluorescence microscope to allow quantification of PPARG % area. As depicted in FIG. 12, immunofluorescence staining and imaging quantification showed a 10.2 fold increase in PPARG stained cells after 2×3 hour exposures separated by 24 hours at the normal incubation temperature, the best performing condition by staining. As depicted in FIG. 13, qPCR data showed an 8.5 fold increase in PPARG transcription levels following heat shock at 43° C. for 6 hours, the best performing condition by qPCR.

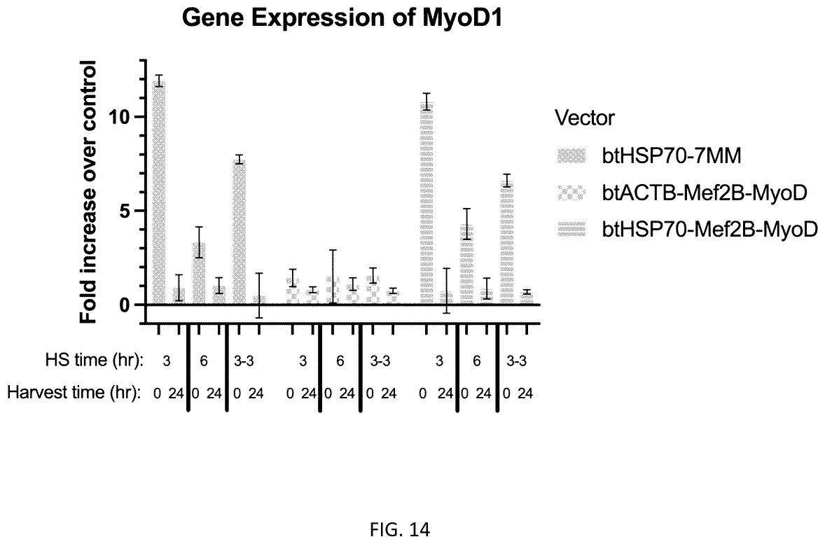

As shown in FIG. 14, heat inducible cell lines were generated in 4CT bovine cells following the same transfection protocols above but with plasmids having either btHSP70-7MM (7MM includes nucleic acids coding for PAX7, MEF2B, and MYOD under control of the btHSP70 promoter), btHSP70-MEF2B-MYOD (construct having nucleic acids encoding MEF2B and MYOD under control of the btHSP70 promoter), or btACTB-MEF2B-MYOD (the control-nucleic acid encoding MEF2B and MYOD under control of the btACTB (beta-actin) promoter).

The latter two plasmids also included an integrated CMV-mRuby sequence to allow for enrichment of cells with integrated constructs. Relative fold change of MYOD mRNA expression was measured immediately after heat shock (i.e. 43° C.) and 24 hours after heat shock across three heat shock conditions of 3 hours, 6 hours, and two separate 3 hour shocks separated by 24 hours at 37° C. The cells treated with 3 hour heat shock conditions performed the best across the test conditions and exhibited peak expression immediately after the 3 hour heat shock and minimal expression just 24 hours later. A 12 fold increase in MYOD mRNA from the btHSP70-7MM vector and an 11 fold increase in MYOD mRNA from the btHSP70-MEF2B-MYOD illustrates strong expression suitable for driving expression of muscle phenotypes associated with these groups of genes. Specifically, btHSP70-7MM showed 12 fold increase of MYOD expression immediately after 3 hours of heat shock, 1 fold increase of MYOD expression 24 hours after 3 hours of heat shock, 3.4 fold increase of MYOD expression immediately after 6 hours of heat shock, 1 fold increase of MYOD expression 24 hours after 6 hours of heat shock, 7.8 fold increase in MYOD expression immediately after 2 rounds of 3 hours of heat shock separated by 24 hours, and 0.5 fold increase in MYOD expression 24 hours after 2 bouts of 3 hours of heat shock separated by 24 hours. While btHSP70-MEF2B-MYOD showed 11 fold increase in MYOD expression immediately after 3 hours of heat shock, 0.8 fold increase in MYOD expression 24 hours after 3 hours of heat shock, 4.5 fold increase of MYOD expression immediately after 6 hours of heat shock, 1 fold increase in MYOD expression 24 hours after 6 hours of heat shock, 6.5 fold increase in MYOD expression immediately after 2 rounds of 3 hours of heat shock separated by 24 hours, and 0.8 fold increase in MYOD expression 24 hours after 2 rounds of 3 hours of heat shock separated by 24 hours. The control beta actin constitutive promoter showed only minimal expression in response to heat shock. Of note, a quick return to normal expression levels, e.g. after 24 hours, as shown by this data, is particularly advantageous when expression is desired for only short durations. For instance, the expression of the genes associated with muscle phenotypes tends to reduce cell proliferation and is only desirable at specific times of a cell culture process that must grow cells to high densities. Ideal times for expression may include shortly before, during, or after complete or partial harvest.

In summary, the bovine HSP70 promoter enabled temperature inducible gene expression in bovine and chicken cells. Heat shocking the cells at 43° C. for 18 hours induced the most gene expression. Leaky (non-induced) expression at lower temperatures can be reduced through optimization and/or single cell cloning as previously described. After heat shock, reporter gene expression returned to baseline over around 6 days (about 24 hour half-life) and cells remained viable and proliferative.

To determine if the heat shock system could induce MYOD protein expression, immunofluorescence was performed. Eight cell lines (naïve control, empty vector control, btHSP70-7MM, btACTB-MEF2B-MYOD, btHSP70-MEF2B-MYOD, btACTB-MEF2B-CMV-mRuby, btHSP70-MEF2B-MYOD-CMV-mRuby, and primary myoblasts) were subjected to different heat shock conditions at 43° C. Protein expression was measured at baseline (0 hours), 3 hours of heat shock, 6 hours of heat shock, 3 rounds of heat shock for 3 hours, and 2 rounds of heat shock for 6 hours. Multiple rounds of heat shock were separated by an incubation period of 24 hours at 37° C. Cells were fixed immediately after heat shock and 24 hours after heat shock (only for 3 hour heat shock plate).

After cells were subjected to heat shock conditions, the media was removed, and cells were washed with PBS. Next, cells were fixed in 4% paraformaldehyde for 10-20 minutes, washed with PBS, and permeabilized with 0.1% PBS-T for 10 minutes. After permeabilization, the cells were incubated with 5% NGS solution in 0.1% of PBS-T for 30 minutes. After blocking, the cells were incubated in primary mouse anti-MYOD antibody solution (1:1000, Santa Cruz Biotechnology, Cat. No. SC-32758) for 1 hour at room temperature. The primary antibody was washed off and a secondary goat anti-mouse Alexa Fluor 488 antibody solution (1:1000, Invitrogen, Cat No. A11001) and DAPI nuclear stain solution (1:500) were added to the cells for 45 minutes at room temperature. Lastly, cells are washed with 0.1% PBS-T and imaged. Cellular analysis and counting were completed using BioTek Cytation 5 Cell Imaging Multimode Reader (Agilent). Automatic threshold was set for DAPI nuclear stain and a threshold of 3600 was set for FITC. The percentage of MYOD positive cells was calculated using the following equation: % MyoD positive cells=(FITC positive cells)/(DAPI stained cells)×100.

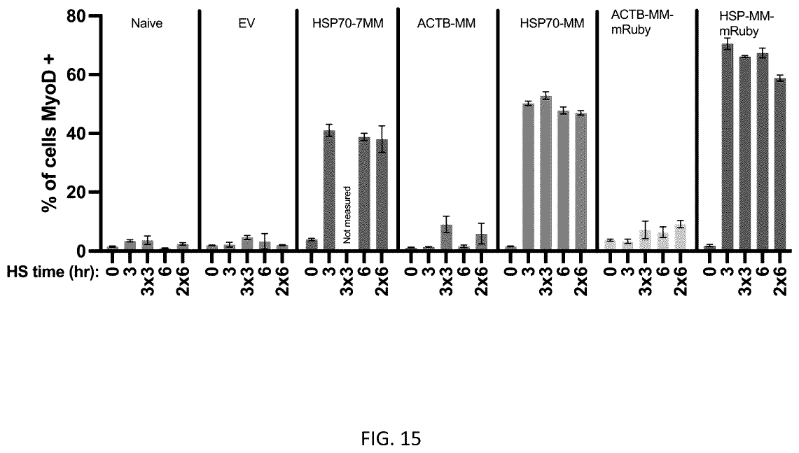

As shown in FIG. 15, when compared to negative control cell lines, btHSP70-7MM, btHSP70-MM, and btHSP-MM-mRuby exhibited a robust increase in MYOD positive cells under all heat shock conditions. In btHSP70-7MM cells, about 40% of cells exhibited MYOD expression across different heat shock conditions. Similarly, about 50% of btHSP70-MM cells showed detectable protein expression of MYOD when subjected to various heat shock conditions. Under 3 hour heat shock, 3 rounds of 3 hour heat shock, and 6 hours of heat shock conditions, btHSP-MM-mRuby about 70% of cells exhibited detectable MYOD expression. In contrast, cell lines with R-actin promoters, btACTB-MM and btACTB-MM-mRuby, showed minimal induction of MYOD protein expression. These results indicate that the heat shock promoter system can induce both gene and protein expression of target molecules. Little to no leaky (non-induced) expression was observed in non-heat shocked HSP cell lines.

Heat inducible bovine myosin heavy chain (MYH2) expressing 4CT cell lines were generated and evaluated for heavy chain myosin mRNA expression under heat shock conditions. Cell lines 4CT Naïve, PBV5-btHSPA1A-btMYH2-1, PBV5-btHSPA1A-MYH2-2, and PBV5-btACTB-MYH2-2 were seeded on 96 well plates, and exposed to four heat shock conditions. The cells were subjected to no heat shock (37° C. for 24 hours), heat shock at 43° C. for 6 hours, followed by incubation at 37° C. for 24 hours, 41° C. for 2 days, and 39° C. for 2 days. Sequences of btMYH2-1 and btMYH2-2 are detailed in Table 2.

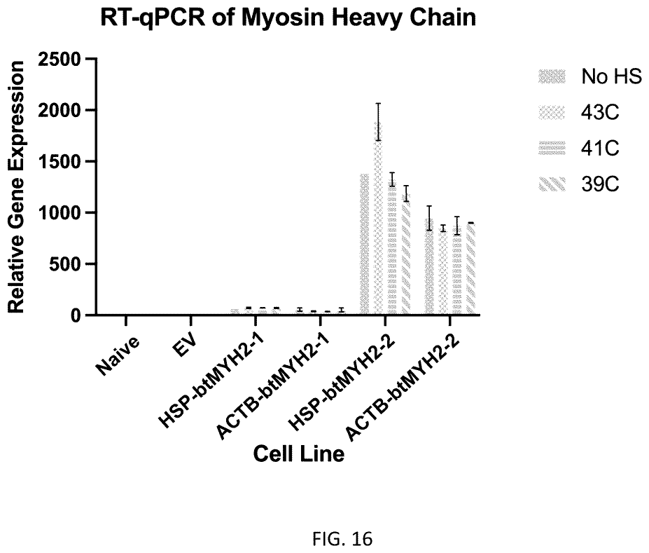

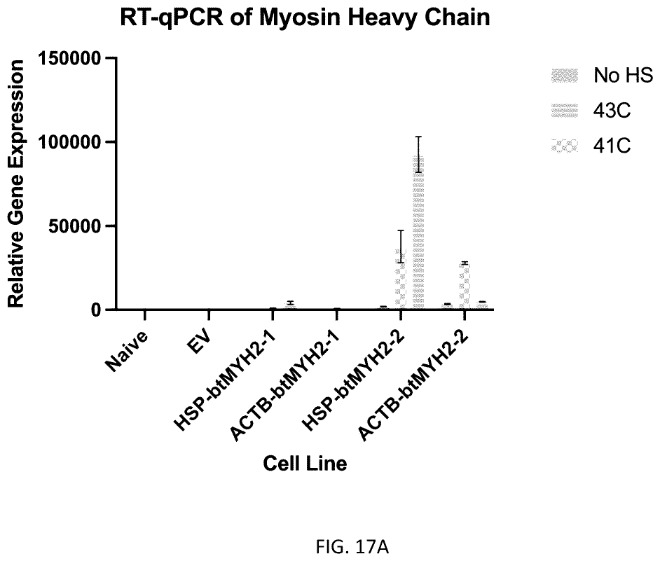

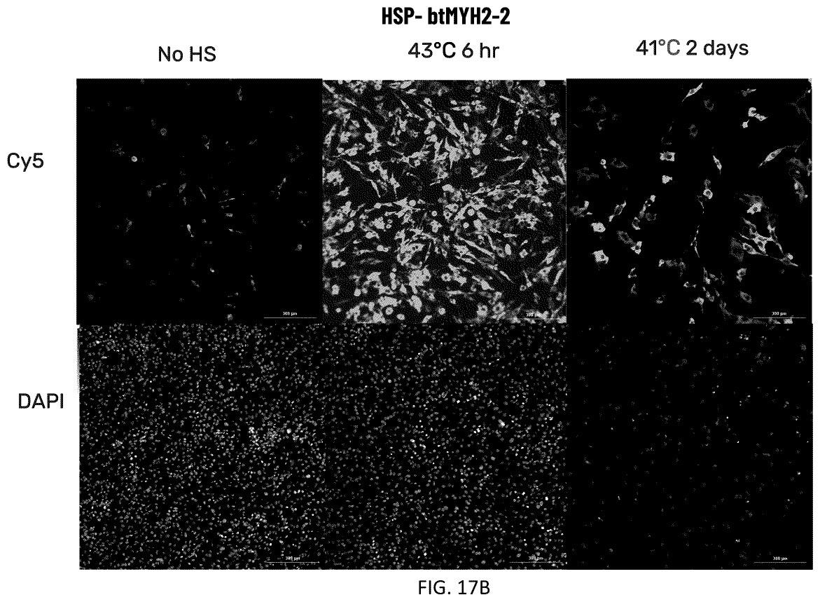

RNA was extracted from the cells and cDNA was generated for the RT-qPCR assay. Bovine PGK1 Bt03225857_m1 was used as the housekeeping gene (positive control) and btMYHC-1 and btMYH2-2 target genes were evaluated. The ΔΔCT method was utilized to determine relative mRNA expression, and two biological replicates and two technical replicates were performed for each condition. All conditions were compared to native cell lines cultured under standard conditions at 37° C. As shown in FIG. 16, heat shock conditions did not significantly impact relative gene expression of myosin heavy chain in btHSP-btMYH2 and btACTB-btMYH2 4CT cell lines, as myosin heavy chain expression remained relatively low. In particular, btHSP-btMYH2-2 exhibited increased myosin heavy chain expression at 43° C. compared to no heat shock. BtHSP-btMYH2-2 and btACTB-btMYH2-2 4CT cell lines both exhibited leaky expression. However, leakiness can be reduced by selecting for clones that exhibit lower levels of target gene expression without heat shock induction. As detailed in FIG. 17A, the Levee cell line, of MSC origin, with btHSP-btMYH2-2 exhibited myosin heavy chain expression at 43° C. nearly 5× higher than 4CT cell line BtHSP-btMYH2-2 of FIG. 16, while advantageously having little leaky expression without heat shock induction. Fluorescent imaging of the Levee btHSP-btMYH2, as shown in FIG. 17B, shows robust myosin expression with Cy5 staining and cell viability with DAPI staining after 6 hours at 43° C. and confirms minimal leaky expression without heat shock induction.

TABLE 2

| | |
|---|---|
| btMYH2-1 | ATGAGTTCGGACCAGGAAATGGCCATCTTTGGGGAGGCTGCTCCTTACCTCCGAAAGTCTGAAA |
| Nucleotide | AGGAGCGCATTGAAGCCCAGAATAAGCCTTTTGATGCCAAGACATCTGTCTTTGTGGCCGAGCC |
| Sequence | CAAGGAATCTTTTGTCAAAGGGACTATCCAGAGCAGAGAAGGTGGGAAAGTGACAGTGAAGACC |
| SEQ ID | GAAGGAGGAGCGACTCTGACAGTGAAAGAGGATCAAGTCTTCCCCATGAATCCTCCCAAATTCG |
| NO: 25 | ACAAGATCGAGGACATGGCCATGATGACCCACTTGCACGAGCCTGCTGTGCTGTACAACCTCAA |
| | AGAGCGTTATGCAGCCTGGATGATCTACACCTACTCGGGCCTCTTCTGCGTCACCGTCAACCCC |
| | TACAAGTGGCTGCCGGTGTACAACCCTGAGGTGGTGACGGCCTACCGAGGCAAAAAGCGCCAGG |
| | AGGCCCCGCCCCACATCTTCTCCATCTCTGACAACGCCTATCAGTTCATGCTGACTGACCGAGA |
| | GAATCAGTCAATCCTGATCACCGGAGAATCCGGGGCAGGAAAGACTGTGAACACAAAACGTGTC |
| | ATCCAGTACTTTGCAACAATTGCAGTCACTGGGGAGAAGAAGAAGGAGGAAATAACTTCAGGCA |
| | AAATACAGGGGACTCTGGAGGATCAGATCATCAGTGCCAATCCACTGCTTGAGGCCTTTGGCAA |
| | CGCCAAGACCGTGAGGAATGACAACTCCTCTCGCTTTGGTAAATTCATTAGAATCCACTTTGGC |
| | ACTACAGGAAAACTGGCCTCTGCTGATATTGAAACATATCTGTTAGAGAAGTCTAGAGTTACTT |
| | TCCAGCTTAAGGCTGAAAGGAGCTATCATATTTTTTACCAGATCACATCAAACAGGAAACCAGA |
| | ACTAATTGAGATGCTTCTGATTACCACCAACCCATATGACTACCCATTCATCAGTCAAGGGGAG |
| | ATCAGTGTGGCCAGTATTGATGATCAGGAAGAACTGATAGCCACAGATAGTGCTATTGATATTT |
| | TGGGCTTTACTAATGAAGAGAAGGTCTCCATCTACAAGCTCACGGGGGCTGTGATGCATTATGG |
| | GAACCTGAAATTCAAGCAAAAGCAGCGTGAGGAGCAAGCAGAGCCAGATGGCACTGAAGTGGCT |
| | GACAAGGCTGCCTACCTCCAGAGTCTGAACTCTGCTGACCTACTCAAAGCCCTTTGCTACCCCA |
| | GGGTCAAGGTTGGCAATGAGTATGTCACCAAAGGCCAGACTGTAGAGCAGGTAACCAATGCAGT |
| | GGGTGCTCTGGCCAAAGCCGTCTATGAGAAGATGTTCCTGTGGATGGTCGCCCGCATCAACCAG |
| | CAGCTGGACACCAAGCAGCCCAGGCAGTACTTCATCGGGGTCTTGGACATCGCTGGCTTTGAGA |
| | TCTTTGATTTCAACAGCCTGGAGCAGCTGTGCATCAACTTCACCAATGAGAAACTGCAACAGTT |
| | TTTCAACCACCACATGTTCGTGCTGGAGCAGGAGGAGTACAAAAGAGAAGGCATCGAGTGGACG |
| | TTCATTGACTTTGGGATGGACCTGGCTGCCTGCATCGAGCTCATTGAGAAGCCTATGGGCATCT |
| | TCTCCATCCTCGAAGAGGAGTGCATGTTCCCCAAGGCCACAGACATGTCCTTCAAGAACAAGCT |
| | GTATGACCAGCACCTGGGCAAGTCTGCCAATTTCCAGAAGCCCAAGGTGGTCAAGGGCAAACCT |
| | GAGGCCCATTTTGCCCTGATCCACTATGCGGGTGTCGTGGACTACAACATTACTGGCTGGCTGG |
| | AGAAGAACAAGGACCCCCTGAACGATACTGTGGTTGGGCTGTACCAGAAGTCTGCGTTGAAAAC |
| | TCTAGCTTTCCTTTTCTCTGGGACTCCAACTGGTGACTCAGAGGCAAGTGGTGGAACTAAGAAA |
| | GGTGGTAAGAAGAAGGGCTCTTCTTTCCAGACGGTGTCTGCACTTTTCAGGGAAAATCTGAACA |
| | AACTGATGACCAACCTCAGGAGTACCCATCCTCACTTTGTGCGGTGCATCATCCCCAATGAAAC |
| | GAAGACCCCTGGGGCCATGGAGCACGAGCTGGTCCTGCACCAGCTGAGGTGTAATGGCGTGCTA |
| | GAGGGCATCCGCATCTGCAGGAAGGGCTTCCCCAGCAGAATCCTGTATGCGGACTTCAAACAGA |
| | GATACAAGGTATTAAATGCAAGTGCGATCCCTGAAGGACAATACATCGACAGCAAGAAGGCTTC |
| | TGAGAAGCTCCTTGCATCCATTGACATCGACCACACCCAGTATAAATTTGGTCACACCAAGGTC |
| | TTTTTCAAAGCTGGTCTTCTGGGGCTCCTAGAGGAGATGCGAGATGAAAAGCTAGCCCAGCTGA |
| | TGACCCGAACCCAGGCCAGGTGCAGAGGGTTCTTGGCAAGAGTGGAGTACCAGAAGATGGTGGA |
| | GAGGAGGGAGTCCATCTTCTGCATCCAGTACAACATCCGAGCCTTCATGAATGTCAAGCACTGG |
| | CCCTGGATGAAACTGTTCTTCAGGATCAAGCCCCTGCTGAAGAGTGCAGAGACGGAGAAGGAGA |
| | TGGCCACCATGAAGGAAGAGTTCCAGAAAACCAAAGATGAACTGGCCAAGTCAGAGGCCAAAAG |
| | GAAGGAACTGGAGGAAAAAATGGTGACTCTCTTGAAAGAAAAAAATGACCTACAGCTCCAGGTT |
| | CAATCTGAAGCTGAAGGCTTGGCTGATGCAGAGGAAAGATGTGACCAGCTGATCAAAACCAAAA |
| | TCCAGCTGGAGGCTAAGATCAAGGAGGTGACTGAGAGAGCAGAGGATGAGGAAGAGATCAATGC |
| | TGAGCTGACGGCCAAGAAGAGGAAACTGGAGGACGAATGTTCGGAACTAAAGAAAGACATAGAC |
| | GACCTTGAGCTGACACTGGCCAAGGTTGAGAAGGAGAAACATGCCACAGAAAATAAGGTGAAAA |
| | ACCTCACAGAAGAGATGGCAGGTCTGGATGAAACCATCGCCAAGCTGACCAAGGAGAAAAAGGC |
| | CCTCCAGGAGGCCCACCAGCAGACCCTGGATGACCTGCAGGCAGAAGAGGACAAAGTCAACACT |
| | CTGACCAAAGCTAAAACCAAGCTAGAGCAGCAAGTGGATGACCTTGAGGGGTCCTTGGAGCAAG |
| | AAAAGAAACTTCGCATGGACCTAGAGAGAGCCAAGAGGAAACTTGAAGGTGATCTCAAGTTGGC |
| | CCAAGAGTCCATAATGGACATTGAAAATGAAAAACAACAACTTGATGAAAAGCTCAAAAAGAAA |
| | GAGTTTGAAATCAGCAATTTGCAAAGCAAGATTGAAGATGAACAAGCACTTGGCATTCAACTGC |
| | AGAAGAAGATCAAAGAGTTGCAGGCCCGCATCGAGGAGCTGGAGGAGGAGATCGAGGCCGAGCG |
| | GGCCTCCCGCGCCAAGGCAGAGAAGCAGCGCTCAGACCTCTCCCGGGAACTGGAGGAGATCAGC |
| | GAGCGGCTAGAAGAAGCCGGCGGGGCCACTTCCGCCCAGATTGAGATGAACAAGAAGCGGGAGG |
| | CCGAGTTCCAGAAGATGCGCAGGGACCTGGAGGAGGCCACCCTGCAGCACGAGGCCACGGCGGC |
| | CGCTCTTCGCAAGAAGCACGCGGACAGTGTGGCTGAGCTGGGGGAGCAGATTGACAACCTGCAG |
| | AGGGTCAAGCAGAAGCTGGAGAAGGAGAAGAGCGAGATGAAGATGGAGATCGACGACCTTGCTA |
| | GCAATGTGGAAACGATCTCTAAAGCCAAGGGAAACCTAGAGAAGATGTGCCGAACTCTGGAGGA |
| | CCAAGTGAACGAGCTGAAATCCAAGGAGGAGGAGCAGCAGCGGCTGATCAATGACCTGACAACC |
| | CAGAGGGGACGCTTGCAGACTGAATCTGGTGAATTTTCACGTCAGCTAGATGAGAAAGAAGCTC |
| | TGGTGTCTCAGTTGTCAAGGGGCAAACAAGCATTTACTCAACAGATTGAGGAATTAAAGAGGCA |
| | ACTTGAAGAGGAGATAAAGGCCAAGAATGCCCTGGCCCATGGCCTGCAGTCAGCCCGTCATGAC |
| | TGTGACCTGCTGCGTGAACAGTATGAGGAGGAGCAGGAATCCAAGGCCGAGCTGCAGAGGGCGC |
| | TGTCCAAGGCCAACACCGAGGTGGCCCAGTGGAGGACCAAGTATGAGACAGACGCCATCCAGCG |
| | CACGGAGGAGCTGGAGGAGGCCAAGAAGAAACTGGCCCAGCGTCTGCAGGCTGCTGAGGAACAC |
| | GTGGAAGCTGTGAACGCCAAATGCGCCTCCCTTGAGAAGACCAAGCAGAGGCTCCAGAATGAGG |
| | TTGAGGACCTCATGCTCGATGTGGAGAGGACAAACGCTGCCTGCGCAGCCCTGGACAAAAAGCA |
| | AAGGAACTTTGACAAGATCCTGGCAGAATGGAAACAGAAGTATGAGGAAACCCACGCCGAGCTC |
| | GAGGCAGCTCAGAAGGAGGCCCGCTCTCTGGGTACCGAGCTGTTCAAGATGAAGAATGCCTACG |
| | AGGAGTCCTTGGATCAGCTAGAAACTTTGAAACGAGAGAACAAAAACTTGCAGCAGGAGATCTC |
| | TGACCTCACGGAGCAGATCGCAGAAGGAGGGAAACGTATGCATGAACTGGAGAAAATAAAGAAG |
| | CAAGTGGAACAAGAAAAGTCTGAAATTCAGGCTGCTTTAGAGGAGGCAGAGGCATCTCTTGAAC |
| | ACGAAGAGGGCAAGATCCTGCGTATCCAGCTGGAGCTGAACCAAGTCAAGTCTGAAATTGACAG |
| | GAAAATTGCTGAAAAGGATGAGGAAATTGACCAGCTGAAGAGGAACCACATTAGAGTCGTGGAG |
| | TCCATGCAGACCATGCTGGATGCTGAAATCAGGAGCAGGAATGACGCCATCAGGCTCAAGAAGA |
| | AGATGGAAGGAGACCTCAATGAAATGGAGATCCAGTTGAACCATGCCAACCGCATGGCTGCTGA |
| | GGCCCTGAAGAACTACAGGAACACTCAAGCCATCCTCAAGGATACCCAGATCCACCTGGATGAT |
| | GCTCTGCGGGGCCAGGAGGACTTGAAGGAGCAGCTGGCCATGGTGGAGCGCAGAGCCAACCTGC |
| | TGCAGGCTGAGATCGAGGAGCTGAGGGCCACCCTGGAGCAGACAGAGAGGAGCAGGAAGATCGC |
| | AGAGCAGGAGCTCCTGGATGCCAGTGAGCGCGTCCAGCTCCTGCACACCCAGAACACCAGCCTG |
| | ATCAACACCAAGAAGAAGCTAGAGACAGACATCACGCAAATCCAGGGAGAGATGGAGGACATTC |
| | TCCAGGAAGCTCGCAACGCAGAAGAGAAAGCCAAGAAGGCCATCACTGATGCAGCCATGATGGC |
| | TGAGGAGCTGAAGAAGGAGCAGGACACCAGCGCCCACCTGGAGCGGATGAAGAAGAACATGGAG |
| | CAGACGGTGAAGGACCTGCAGAACCGTCTGGATGAGGCTGAGCAGCTGGCCCTGAAGGGCGGGA |
| | AGAAGCAAATCCAGAAACTGGAGGCCAGGGTGCGTGAGCTGGAAGGAGAGGTAGAGAGTGAGCA |
| | GAAGCGCAATGTGGAAGCTGTCAAGGGTCTGCGCAAACACGAGAGGAGAGTGAAGGAACTCACT |
| | TACCAGACGGAAGAAGATCGGAAAAATATTCTCAGACTTCAGGACTTGGTAGATAAACTTCAGG |
| | CGAAAGTGAAATCTTACAAGAGACAAGCTGAGGAGGCTGAGGAACAATCCAATACAAATCTATC |
| | TAAATTCCGCAAGCTCCAGCATGAGCTGGAGGAAGCCGAGGAAAGGGCTGACATTGCTGAATCC |
| | CAGGTCAACAAGCTGCGGGTGAAGAGCCGGGAGGTTCACACAAAAATCATCAGTGAAGAGTGA |
| btMYH2-1 | MSSDQEMAIFGEAAPYLRKSEKERIEAQNKPFDAKTSVEVAEPKESFVKGTIQSREGGKVTVKT |
| Amino | EGGATLTVKEDQVFPMNPPKFDKIEDMAMMTHLHEPAVLYNLKERYAAWMIYTYSGLFCVTVNP |
| Acid | YKWLPVYNPEVVTAYRGKKRQEAPPHIFSISDNAYQFMLTDRENQSILITGESGAGKTVNTKRV |
| Sequence | IQYFATIAVTGEKKKEEITSGKIQGTLEDQIISANPLLEAFGNAKTVRNDNSSRFGKFIRIHFG |
| SEQ ID | TTGKLASADIETYLLEKSRVTFQLKAERSYHIFYQITSNRKPELIEMLLITTNPYDYPFISQGE |
| NO: 26 | ISVASIDDQEELIATDSAIDILGFTNEEKVSIYKLTGAVMHYGNLKFKQKQREEQAEPDGTEVA |
| | DKAAYLQSLNSADLLKALCYPRVKVGNEYVTKGQTVEQVTNAVGALAKAVYEKMFLWMVARINQ |
| | QLDTKQPRQYFIGVLDIAGFEIFDENSLEQLCINFTNEKLQQFFNHHMFVLEQEEYKREGIEWT |
| | FIDFGMDLAACIELIEKPMGIFSILEEECMFPKATDMSFKNKLYDQHLGKSANFQKPKVVKGKP |
| | EAHFALIHYAGVVDYNITGWLEKNKDPLNDTVVGLYQKSALKTLAFLFSGTPTGDSEASGGTKK |
| | GGKKKGSSFQTVSALFRENLNKLMTNLRSTHPHFVRCIIPNETKTPGAMEHELVLHQLRCNGVL |
| | EGIRICRKGFPSRILYADFKQRYKVLNASAIPEGQYIDSKKASEKLLASIDIDHTQYKFGHTKV |
| | FFKAGLLGLLEEMRDEKLAQLMTRTQARCRGFLARVEYQKMVERRESIFCIQYNIRAFMNVKHW |
| | PWMKLFFRIKPLLKSAETEKEMATMKEEFQKTKDELAKSEAKRKELEEKMVTLLKEKNDLQLQV |
| | QSEAEGLADAEERCDQLIKTKIQLEAKIKEVTERAEDEEEINAELTAKKRKLEDECSELKKDID |
| | DLELTLAKVEKEKHATENKVKNLTEEMAGLDETIAKLTKEKKALQEAHQQTLDDLQAEEDKVNT |
| | LTKAKTKLEQQVDDLEGSLEQEKKLRMDLERAKRKLEGDLKLAQESIMDIENEKQQLDEKLKKK |
| | EFEISNLQSKIEDEQALGIQLQKKIKELQARIEELEEEIEAERASRAKAEKQRSDLSRELEEIS |
| | ERLEEAGGATSAQIEMNKKREAEFQKMRRDLEEATLQHEATAAALRKKHADSVAELGEQIDNLQ |
| | RVKQKLEKEKSEMKMEIDDLASNVETISKAKGNLEKMCRTLEDQVNELKSKEEEQQRLINDLTT |
| | QRGRLQTESGEFSRQLDEKEALVSQLSRGKQAFTQQIEELKRQLEEEIKAKNALAHGLQSARHD |
| | CDLLREQYEEEQESKAELQRALSKANTEVAQWRTKYETDAIQRTEELEEAKKKLAQRLQAAEEH |
| | VEAVNAKCASLEKTKQRLQNEVEDLMLDVERTNAACAALDKKQRNFDKILAEWKQKYEETHAEL |
| | EAAQKEARSLGTELFKMKNAYEESLDQLETLKRENKNLQQEISDLTEQIAEGGKRMHELEKIKK |
| | QVEQEKSEIQAALEEAEASLEHEEGKILRIQLELNQVKSEIDRKIAEKDEEIDQLKRNHIRVVE |
| | SMQTMLDAEIRSRNDAIRLKKKMEGDLNEMEIQLNHANRMAAEALKNYRNTQAILKDTQIHLDD |
| | ALRGQEDLKEQLAMVERRANLLQAEIEELRATLEQTERSRKIAEQELLDASERVQLLHTQNTSL |
| | INTKKKLETDITQIQGEMEDILQEARNAEEKAKKAITDAAMMAEELKKEQDTSAHLERMKKNME |
| | QTVKDLQNRLDEAEQLALKGGKKQIQKLEARVRELEGEVESEQKRNVEAVKGLRKHERRVKELT |
| | YQTEEDRKNILRLQDLVDKLQAKVKSYKRQAEEAEEQSNTNLSKFRKLQHELEEAEERADIAES |
| | QVNKLRVKSREVHTKIISEE |
| btMYH2-2 | ATGAGCAGCGACCAAGAAATGGCGATATTTGGCGAGGCCGCCCCCTACCTGAGAAAGAGCGAGA |
| Nucleotide | AGGAGAGAATCGAGGCTCAGAACAAGCCCTTCGACGCCAAGACAAGCGTGTTCGTGGCCGAGCC |
| Sequence | CAAGGAGAGCTTCGTGAAGGGCACCATTCAGAGCAGAGAAGGCGGTAAAGTGACGGTGAAGACC |
| SEQ ID | GAGGGCGGCGCCACCTTAACTGTAAAGGAGGACCAAGTGTTCCCCATGAACCCCCCCAAGTTCG |
| NO: 27 | ATAAAATCGAGGACATGGCCATGATGACCCACCTGCACGAGCCCGCCGTGCTGTACAACCTGAA |
| | GGAGAGATACGCCGCCTGGATGATCTACACCTACAGCGGCCTGTTCTGCGTGACCGTGAACCCC |
| | TACAAGTGGCTGCCCGTGTACAACCCCGAGGTGGTGACCGCCTACAGAGGCAAGAAGAGACAAG |
| | AGGCCCCCCCCCACATCTTTTCTATCAGCGATAACGCCTATCAGTTCATGCTGACCGACAGAGA |
| | GAATCAATCTATCCTCATCACCGGCGAGAGCGGCGCCGGCAAGACCGTGAACACCAAGAGAGTG |
| | ATTCAGTACTTCGCCACCATCGCCGTGACCGGCGAGAAGAAGAAAGAAGAGATAACGAGCGGCA |
| | AGATCCAAGGCACTCTCGAGGACCAAATTATTAGCGCTAACCCCCTGCTGGAGGCCTTCGGCAA |
| | CGCCAAGACCGTGAGAAACGACAATAGCTCGCGGTTTGGCAAGTTCATCAGAATCCACTTCGGC |
| | ACCACCGGCAAGCTGGCTAGCGCCGACATCGAGACCTACCTGCTGGAGAAGAGCAGAGTGACCT |
| | TTCAGCTGAAGGCCGAGAGAAGCTACCACATCTTCTATCAGATCACAAGCAACAGAAAGCCCGA |
| | ACTCATAGAGATGTTACTGATCACCACCAACCCCTACGACTACCCCTTCATCAGCCAAGGGGAG |
| | ATCAGCGTGGCTAGCATTGACGACCAAGAGGAGCTGATCGCCACCGACAGCGCCATCGACATCC |
| | TGGGCTTCACCAACGAAGAGAAGGTGAGCATCTACAAGCTGACCGGCGCCGTGATGCACTACGG |
| | CAACCTGAAATTCAAACAGAAACAGAGAGAGGAGCAAGCCGAGCCCGACGGCACTGAAGTGGCC |
| | GATAAGGCCGCCTACCTGCAGAGCCTGAACAGCGCCGACCTGCTGAAGGCCCTGTGCTACCCAA |
| | GAGTGAAGGTGGGCAACGAGTACGTGACCAAGGGACAGACCGTGGAGCAAGTGACCAACGCCGT |
| | GGGCGCACTGGCCAAAGCCGTTTACGAGAAGATGTTCCTGTGGATGGTGGCTAGAATCAACCAA |
| | CAACTGGACACTAAGCAGCCAAGACAGTACTTCATCGGAGTGCTTGACATCGCCGGCTTCGAGA |
| | TCTTCGACTTCAACTCGTTAGAACAACTGTGCATCAACTTTACGAACGAAAAGTTACAGCAGTT |
| | CTTCAACCACCACATGTTCGTGCTTGAGCAAGAGGAATATAAGAGAGAGGGCATCGAGTGGACC |
| | TTCATCGACTTCGGCATGGACCTGGCCGCCTGCATCGAGCTTATAGAAAAGCCAATGGGCATCT |
| | TCAGCATTCTGGAAGAAGAGTGCATGTTCCCCAAGGCCACCGACATGAGCTTCAAGAACAAGCT |
| | GTACGATCAGCACCTGGGCAAGAGCGCCAACTTTCAGAAGCCCAAGGTGGTGAAGGGCAAGCCC |
| | GAGGCCCACTTCGCCCTGATCCACTACGCCGGCGTGGTGGACTACAACATCACCGGCTGGCTGG |
| | AGAAAAACAAGGACCCACTGAACGACACCGTGGTGGGCCTGTATCAGAAGAGCGCCCTGAAGAC |
| | TCTGGCCTTCCTGTTCTCCGGCACCCCCACCGGCGACTCGGAGGCTAGCGGCGGCACCAAGAAG |
| | GGCGGTAAAAAAAAAGGCAGCAGCTTTCAGACCGTGAGCGCCCTGTTCAGAGAGAACCTGAATA |
| | AACTGATGACCAACTTGAGAAGCACCCACCCCCACTTCGTGAGATGCATCATCCCCAACGAGAC |
| | GAAAACACCGGGCGCCATGGAGCACGAGTTAGTGCTGCATCAGCTGAGATGCAACGGCGTACTG |
| | GAGGGCATCAGAATCTGCAGAAAGGGCTTCCCAAGCAGAATCCTGTACGCCGACTTCAAGCAGA |
| | GATACAAGGTGCTGAACGCTAGCGCCATCCCCGAGGGACAGTACATCGACAGCAAGAAGGCTAG |
| | CGAGAAGCTGTTAGCTAGCATCGACATCGACCACACACAGTACAAGTTCGGCCACACCAAGGTG |
| | TTCTTCAAGGCCGGCCTGCTGGGCCTGCTAGAAGAGATGCGTGACGAAAAACTCGCCCAATTAA |
| | TGACAAGAACGCAAGCTAGATGCAGAGGCTTCCTGGCTAGAGTGGAGTATCAGAAGATGGTGGA |
| | GAGAAGAGAGAGTATCTTCTGCATTCAGTACAACATCAGAGCCTTCATGAACGTGAAGCACTGG |
| | CCCTGGATGAAGCTGTTCTTCAGAATCAAGCCCCTGCTGAAGAGCGCCGAGACCGAGAAAGAGA |
| | TGGCCACGATGAAGGAGGAGTTTCAGAAGACAAAAGACGAGCTGGCCAAGAGCGAGGCTAAGAG |
| | AAAGGAGCTAGAGGAGAAAATGGTGACCCTGCTGAAGGAGAAGAACGACCTGCAGCTGCAAGTG |
| | CAGAGCGAGGCCGAGGGCCTGGCCGATGCCGAGGAACGATGTGATCAGCTGATCAAGACCAAGA |
| | TTCAGTTGGAGGCCAAGATTAAAGAAGTGACCGAGAGAGCCGAGGATGAAGAGGAGATCAATGC |
| | CGAGCTGACCGCCAAGAAACGGAAGCTCGAAGACGAGTGCAGCGAACTGAAGAAGGACATAGAT |
| | GACTTAGAGCTGACGTTGGCCAAGGTCGAGAAGGAGAAGCATGCCACCGAGAACAAGGTGAAGA |
| | ACCTGACCGAGGAGATGGCCGGCCTGGACGAGACCATCGCCAAGCTGACCAAGGAGAAGAAGGC |
| | CCTTCAAGAGGCCCATCAGCAGACCCTGGACGACCTGCAAGCCGAAGAGGATAAAGTAAACACC |
| | CTGACCAAGGCCAAGACCAAGCTGGAGCAGCAAGTGGACGACCTGGAGGGCAGTCTGGAACAAG |
| | AGAAGAAGCTCAGAATGGACCTGGAGAGAGCCAAGCGAAAATTGGAGGGTGATTTGAAACTGGC |
| | CCAAGAGAGCATCATGGACATCGAGAACGAGAAGCAGCAACTTGATGAAAAATTAAAGAAAAAG |
| | GAGTTCGAGATCAGCAACCTGCAAAGTAAGATTGAAGACGAACAAGCGCTGGGCATTCAGCTGC |
| | AGAAGAAGATTAAGGAGTTACAAGCTAGAATTGAAGAACTCGAGGAAGAAATCGAAGCCGAGAG |
| | GGCTAGCCGCGCCAAGGCCGAGAAACAAAGATCGGATCTGAGCAGAGAACTAGAAGAGATCTCT |
| | GAAAGACTGGAAGAGGCCGGGGGGGCCACGTCAGCTCAGATCGAGATGAACAAGAAGAGAGAGG |
| | CCGAGTTCCAAAAAATGAGAAGAGACCTCGAGGAGGCGACGCTTCAACATGAAGCTACCGCCGC |
| | CGCCCTGAGAAAGAAGCACGCCGACAGCGTGGCCGAGCTGGGCGAGCAGATCGACAACCTGCAG |
| | AGAGTGAAGCAGAAGTTGGAGAAAGAAAAGTCGGAAATGAAGATGGAGATCGACGATCTGGCTA |
| | GCAACGTGGAGACCATCAGCAAGGCCAAGGGCAACCTGGAGAAGATGTGCAGAACCCTGGAAGA |
| | CCAAGTGAACGAACTGAAGAGCAAGGAAGAGGAACAGCAGAGGCTGATCAACGACCTGACCACA |
| | CAGAGAGGCAGACTGCAGACCGAGAGCGGCGAGTTCAGCAGACAGCTGGACGAGAAGGAGGCCC |
| | TGGTGTCTCAGCTGAGCAGAGGCAAGCAAGCCTTCACACAGCAGATCGAGGAGCTGAAAAGACA |
| | GCTTGAAGAAGAGATAAAGGCAAAGAACGCCCTGGCCCACGGCCTGCAGAGCGCTAGACACGAC |
| | TGCGACCTGCTGAGAGAGCAGTATGAAGAGGAACAAGAGTCGAAAGCCGAGCTGCAGAGAGCCC |
| | TGAGCAAGGCCAACACCGAGGTCGCTCAATGGAGAACCAAGTACGAGACCGACGCCATTCAGAG |
| | AACCGAGGAGCTCGAAGAAGCCAAAAAAAAGCTGGCTCAACGACTGCAAGCCGCTGAGGAGCAC |
| | GTTGAGGCAGTGAACGCCAAGTGCGCATCACTGGAGAAAACCAAGCAGCGTCTGCAAAACGAAG |
| | TGGAGGACCTGATGCTGGACGTGGAGAGAACCAACGCCGCCTGCGCCGCCCTGGACAAGAAGCA |
| | GAGAAACTTCGACAAGATCCTGGCCGAGTGGAAGCAGAAGTACGAGGAGACCCACGCCGAGCTG |
| | GAGGCCGCTCAGAAAGAGGCCCGAAGCCTGGGCACCGAGCTGTTCAAGATGAAGAACGCCTACG |
| | AGGAGAGCCTGGATCAGCTGGAGACCCTGAAGAGAGAGAACAAGAACCTGCAGCAAGAGATCTC |
| | TGACTTGACCGAGCAGATCGCCGAGGGTGGTAAGAGGATGCATGAGTTGGAGAAAATCAAGAAG |
| | CAAGTCGAACAAGAGAAAAGTGAGATCCAAGCCGCCCTGGAAGAGGCGGAAGCATCGCTCGAAC |
| | ATGAAGAGGGCAAGATCCTGAGGATTCAGCTGGAGCTCAACCAAGTGAAGAGCGAGATCGACAG |
| | AAAGATTGCCGAGAAAGACGAGGAGATTGATCAGCTGAAGAGAAACCACATCAGAGTGGTGGAG |
| | AGCATGCAGACCATGCTGGACGCCGAGATCAGAAGCAGAAACGACGCCATCAGACTGAAGAAGA |
| | AGATGGAGGGCGACCTGAATGAAATGGAGATTCAGCTGAACCACGCCAACAGAATGGCCGCCGA |
| | GGCCCTGAAGAACTACAGAAACACCCAAGCCATCCTGAAGGACACACAGATTCACCTGGACGAC |
| | GCCCTGAGAGGCCAAGAGGACCTGAAGGAGCAGCTGGCCATGGTGGAACGAAGAGCTAACCTGC |
| | TGCAAGCCGAGATAGAAGAGCTCCGAGCCACCCTGGAGCAAACAGAACGGAGCAGAAAAATCGC |
| | CGAGCAAGAGCTGCTGGACGCTAGCGAGAGAGTGCAACTGCTGCATACACAGAACACAAGCCTG |
| | ATCAACACCAAGAAGAAACTGGAGACCGACATCACGCAGATCCAAGGCGAGATGGAAGACATCC |
| | TCCAAGAGGCTAGAAATGCCGAAGAGAAGGCCAAGAAGGCCATCACCGACGCCGCCATGATGGC |
| | CGAAGAGCTGAAAAAAGAGCAAGACACGTCCGCGCACCTAGAGAGAATGAAGAAGAACATGGAG |
| | CAGACCGTGAAGGATCTGCAGAACAGACTGGACGAGGCCGAGCAACTGGCCCTGAAGGGGGGAA |
| | AGAAACAGATTCAGAAACTTGAAGCAAGGGTGCGTGAGTTAGAGGGCGAGGTGGAGAGCGAGCA |
| | GAAGAGAAACGTTGAGGCGGTGAAGGGTCTGAGAAAGCACGAGAGAAGAGTGAAGGAGCTGACC |
| | TATCAGACCGAAGAAGACCGCAAAAACATCCTGAGACTGCAAGACCTGGTGGACAAGCTGCAAG |
| | CCAAGGTGAAGAGCTACAAGAGACAAGCCGAGGAGGCAGAAGAGCAATCGAACACCAACCTGAG |
| | CAAGTTCAGAAAGCTGCAACACGAGCTGGAGGAAGCGGAGGAAAGAGCCGACATCGCCGAGAGC |
| | CAAGTGAACAAGCTGAGGGTGAAAAGTAGAGAGGTACATACCAAGATAATCAGCGAGGAGTGA |
| btMYH2-2 | MSSDQEMAIFGEAAPYLRKSEKERIEAQNKPFDAKTSVFVAEPKESFVKGTIQSREGGKVTVKT |
| Amino | EGGATLTVKEDQVFPMNPPKFDKIEDMAMMTHLHEPAVLYNLKERYAAWMIYTYSGLFCVTVNP |
| Acid | YKWLPVYNPEVVTAYRGKKRQEAPPHIFSISDNAYQFMLTDRENQSILITGESGAGKTVNTKRV |
| Sequence | IQYFATIAVTGEKKKEEITSGKIQGTLEDQIISANPLLEAFGNAKTVRNDNSSRFGKFIRIHFG |
| SEQ ID | TTGKLASADIETYLLEKSRVTFQLKAERSYHIFYQITSNRKPELIEMLLITTNPYDYPFISQGE |
| NO: 28 | ISVASIDDQEELIATDSAIDILGFTNEEKVSIYKLTGAVMHYGNLKFKQKQREEQAEPDGTEVA |
| | DKAAYLQSLNSADLLKALCYPRVKVGNEYVTKGQTVEQVTNAVGALAKAVYEKMFLWMVARINQ |
| | QLDTKQPRQYFIGVLDIAGFEIFDFNSLEQLCINFTNEKLQQFFNHHMFVLEQEEYKREGIEWT |
| | FIDFGMDLAACIELIEKPMGIFSILEEECMFPKATDMSFKNKLYDQHLGKSANFQKPKVVKGKP |
| | EAHFALIHYAGVVDYNITGWLEKNKDPLNDTVVGLYQKSALKTLAFLFSGTPTGDSEASGGTKK |
| | GGKKKGSSFQTVSALFRENLNKLMTNLRSTHPHFVRCIIPNETKTPGAMEHELVLHQLRCNGVL |
| | EGIRICRKGFPSRILYADFKQRYKVLNASAIPEGQYIDSKKASEKLLASIDIDHTQYKFGHTKV |
| | FFKAGLLGLLEEMRDEKLAQLMTRTQARCRGFLARVEYQKMVERRESIFCIQYNIRAFMNVKHW |
| | PWMKLFFRIKPLLKSAETEKEMATMKEEFQKTKDELAKSEAKRKELEEKMVTLLKEKNDLQLQV |
| | QSEAEGLADAEERCDQLIKTKIQLEAKIKEVTERAEDEEEINAELTAKKRKLEDECSELKKDID |
| | DLELTLAKVEKEKHATENKVKNLTEEMAGLDETIAKLTKEKKALQEAHQQTLDDLQAEEDKVNT |
| | LTKAKTKLEQQVDDLEGSLEQEKKLRMDLERAKRKLEGDLKLAQESIMDIENEKQQLDEKLKKK |
| | EFEISNLQSKIEDEQALGIQLQKKIKELQARIEELEEEIEAERASRAKAEKQRSDLSRELEEIS |
| | ERLEEAGGATSAQIEMNKKREAEFQKMRRDLEEATLQHEATAAALRKKHADSVAELGEQIDNLQ |
| | RVKQKLEKEKSEMKMEIDDLASNVETISKAKGNLEKMCRTLEDQVNELKSKEEEQQRLINDLTT |
| | QRGRLQTESGEFSRQLDEKEALVSQLSRGKQAFTQQIEELKRQLEEEIKAKNALAHGLQSARHD |
| | CDLLREQYEEEQESKAELQRALSKANTEVAQWRTKYETDAIQRTEELEEAKKKLAQRLQAAEEH |
| | VEAVNAKCASLEKTKQRLQNEVEDLMLDVERTNAACAALDKKQRNFDKILAEWKQKYEETHAEL |
| | EAAQKEARSLGTELFKMKNAYEESLDQLETLKRENKNLQQEISDLTEQIAEGGKRMHELEKIKK |
| | QVEQEKSEIQAALEEAEASLEHEEGKILRIQLELNQVKSEIDRKIAEKDEEIDQLKRNHIRVVE |
| | SMQTMLDAEIRSRNDAIRLKKKMEGDLNEMEIQLNHANRMAAEALKNYRNTQAILKDTQIHLDD |
| | ALRGQEDLKEQLAMVERRANLLQAEIEELRATLEQTERSRKIAEQELLDASERVQLLHTQNTSL |
| | INTKKKLETDITQIQGEMEDILQEARNAEEKAKKAITDAAMMAEELKKEQDTSAHLERMKKNME |
| | QTVKDLQNRLDEAEQLALKGGKKQIQKLEARVRELEGEVESEQKRNVEAVKGLRKHERRVKELT |
| | YQTEEDRKNILRLQDLVDKLQAKVKSYKRQAEEAEEQSNTNLSKFRKLQHELEEAEERADIAES |
| | QVNKLRVKSREVHTKIISEE |
| CEBPA | ATGGAGTCGGCCGACTTCTACGAGGCGGAGCCGCGGCCCCCGATGAGCAGCCACCTCCAGAGCC |
| Nucleic | CTCCACACGCTCCTAGCAGCGCAGCTTTCGGCTTTCCTAGAGGCGCTGGCCCTAGCCAGCCACC |
| Acid | TGCCCCACCTGCCGCCCCTGAGCCTCTGGGCGGCATCTGCGAACACGAGACGTCCATCGACATC |
| Sequence | AGCGCCTACATCGACCCGGCCGCCTTCAACGACGAGTTCCTGGCCGACCTGTTCCAACACAGCC |
| (SEQ ID | GGCAGCAGGAGAAGGCCAAGGCTGCAGCTGCACCTGCAGGAGGCGGAAACGACTTTGACTACCC |
| NO: 30) | TGGCGCCCCTGTGGGCCCTGGAGGCGCCGTCATGCCTGGAGGGACACACGGTCCCCCTCCTGGC |
| | TACGGCTGCGCTGCAGCTGGCTACCTGGACAGCAGGCTGGAGCCTCTGTACGAGCGGGTCGGGG |
| | CGCCGGCGCTGCGGCCGCTGGTGATCAAGCAGGAGCCGCGCGAGGAGGACGAAGCTAAGCAGCT |
| | GGCTCTGGCTGGACTCTTTCCTTACCAGCCACCTCCACCTCCACCTCCACCTCACAGCCACCCT |
| | CCACCTGCTCACCTGGCCGCTCCTCACCTGCAGTTCCAGATCGCACACTGCGGCCAGACCACCA |
| | TGCACCTGCAGCCTGGCCACCCTGCTCCTCCACCTACACCTGTGCCTAGCCCTCACCCAGCTCC |
| | TGCACTCGGAGCCGCTGGCCTGCCAGGACCTGGAGGCGCTCTCAAGGGGCTGGCCGCCACACAC |
| | CCCGACCTCCGTGCTGGAGGCGGAGGGGGAGGCAAAGCCAAGAAGTCCGTGGACAAGAACAGCA |
| | ACGAGTACAGGGTGAGGCGCGAGCGCAACAACATCGCGGTGCGCAAGAGCCGGGACAAGGCCAA |
| | GCAGCGCAACGTGGAGACGCAGCTGAAGGTGCTGGAGCTGACCAGTGACAATGACCGCCTGCGC |
| | AAGCGGGTGGAACAACTGAGCCGCGAACTGGACACGCTGCGGGGCACCTTCCGTCAGCTGCCCG |
| | AGAGCTCCCTGGTCAAGGCCATGGGCAACTGCGCGTGA |
| CEBPA | MESADFYEAEPRPPMSSHLQSPPHAPSSAAFGFPRGAGPSQPPAPPAAPEPLGGICEHETSIDI |
| Amino | SAYIDPAAFNDEFLADLFQHSRQQEKAKAAAAPAGGGNDFDYPGAPVGPGGAVMPGGTHGPPPG |
| Acid | YGCAAAGYLDSRLEPLYERVGAPALRPLVIKQEPREEDEAKQLALAGLFPYQPPPPPPPPHSHP |
| Sequence | PPAHLAAPHLQFQIAHCGQTTMHLQPGHPAPPPTPVPSPHPAPALGAAGLPGPGGALKGLAATH |
| (SEQ ID | PDLRAGGGGGGKAKKSVDKNSNEYRVRRERNNIAVRKSRDKAKQRNVETQLKVLELTSDNDRLR |
| NO: 31) | KRVEQLSRELDTLRGTFRQLPESSLVKAMGNCA |

### 7. EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

We claim:

1. A non-human animal cell cultivation method, comprising:
a. cultivating avian, bovine, or non-human mammalian cells comprising a nucleic acid encoding a factor or biomarker associated with a fat or muscle phenotype operably linked to a heat shock promoter in media in a bioreactor for a first time period at a proliferation temperature;
b. exposing the cells to a heat shock temperature for a second time period, thereby inducing expression of the factor or biomarker;
c. separating at least a portion of the heat shocked cells from the media; and
d. harvesting the separated, heat shocked cells for formulation into a comestible food product.

2. The method of claim 1, wherein the cells are cultivated at a proliferation temperature between 35° C. and 40° C. to a target cell density.

3. The method of claim 2, the cells are cultivated to a target cell density of about 0.1 mil/mL cells to about 70 mil/mL.

4. The method of claim 1, wherein the cells are exposed to a heat shock temperature at or between 37° C. and 43° C. and the second time period is about 3 hours, and wherein the heat shock temperature is at least 3° C. higher than the proliferation temperature.

5. The method of claim 1, wherein a substantial portion of the cellular population exhibits expression of the nucleic acid operably linked to the heat shock promoter at 43° C.

6. The method of claim 1, wherein less than 10% of the cells express the nucleic acid operably linked to the heat shock promoter at or below 37° C.

7. The method of claim 1, wherein the cells have a basal doubling time of 24 hours, and wherein the cells return to the basal doubling time within 6 days of exposure to the heat shock temperature.

8. The method of claim 1, wherein the heat shock promoter is a HSP1A1 promoter.

9. The method of claim 1, wherein the heat shock promoter is homologous to the species of the cells and comprises one or more portions of the heat shock promoter having heat-inducible promoter activity.

10. The method of claim 1, wherein the heat shock promoter is modified to exhibit reduced activation by heavy metals or ultraviolet light as compared to a native heat shock promoter.

11. The method of claim 1, wherein the factor or biomarker associated with a fat phenotype is PPARG and/or CEBPA or the factor or biomarker associated with a muscle phenotype is PAX7, MYOD, MYH2 and/or MEF2B (MERFs).

12. The method of claim 1, wherein step b is carried out in a differentiation system for the second time period, wherein the differentiation system comprises culturing the cells in a tank or in a pipe through which the cells flow.

13. The method of claim 1, wherein after step b, the cells are cultivated for a finishing time period, at a proliferation time between 1 and 14 days.

14. The method of claim 1, wherein prior to step d, the cells exhibit a 0.5 fold to 30 fold increase in expression of factors or biomarkers associated with a fat or muscle phenotype compared to non-heat shocked cells.

15. The method of claim 1, wherein prior to step d, the cells exhibit phenotypic and genotypic characteristics of adipocytes and/or myotubes.

16. A chicken or bovine fibroblast cell line, characterized by:
a. suspension adaptation;
b. a population doubling level exceeding 100;
c. a doubling time less than 24 hours;
d. a culture density tolerance exceeding 1×10⁷ cells/ml;
e. adaptation to serum free media;
f. edibility; and
g. heat shock inducible gene expression.

17. The cell line of claim 16, wherein the cell line returns to a doubling time of less than 24 hours 6 days or less after exposure to a heat shock temperature for a time length between about 1 and 6 hours.

18. The cell line of claim 16, wherein heat shock inducible gene expression is induced at a temperature at or between 37° C. and 43° C.

19. The cell line of claim 16, wherein the inducible gene expression comprises one or more genes associated with a fat or muscle phenotype operably linked to a heat shock promoter.

20. The cell line of claim 19, wherein the factor or biomarker associated with a fat phenotype is PPARG and/or CEBPA or the factor or biomarker associated with a muscle phenotype is PAX7, MYOD, MYH2 and/or MEF2B (MERFs).


SEQUENCE LISTING (31 sequences)

SEQ ID NO: 1 - protein, 299 aa, Gallus gallus
MDLLGPMEMTEGSLCSFTAADDFYDDPCFNTSDMHFFEDLDPRLVHVGGLLKPEEHPHHHGHHHGNPHEEEHVRAPSGHHQAGRCLLWACKACKRKTTNADRRKAATMRERRRLSKVNEAFETLKRCTSTNPNQRLPKVEILRNAIRYIESLQALLREQEDAYYPVLEHYSGESDASSPRSNCSDGMMEYSGPPCSSRRRNSYDSSYYTESPNDPKHGKSSVVSSLDCLSSIVERISTDNSTCPILPPAEAVAEGSPCSPQEGANLSDSGAQIPSPTNCTPLPQESSSSSSSNPIYQVL

SEQ ID NO: 2 - protein, 297 aa, Anas platyrhynchos
MDLLGAMEMPEGSLCSFTGADDFYDDPCFNTSDMHFFEDLDPRLVHVGGLLKPEEHPHHHGHHHHGHPHEEEHVRAPSGHHQAGRCLLWACKACKRKTTNADRRKAATMRERRRLSKVNEAFETLKRCTSTNPNQRLPKVEILRNAIRYIESLQALLREQEDVYYPVLEHYSGESDASSPRSNCSDGMMEYSGPPCSSRRRNSYDSSYYTESPNDPKHGKSSVVSSLDCLSGIVERISTDNSTCPILPPAETVAEGSPCSPQEGASLNDSGAQIPSPTNCTPLPQESSSSNPIYQVL

SEQ ID NO: 3 - protein, 297 aa, Meleagris gallopavo
MDLLGPMEMTEGSLCSFAAADDFYDDPCFNTSDMHFFEDLDPRLVHVGGLLKPEEHPHHHGHHHGHPHEEEHVRAPSGHHQAGRCLLWACKACKRKTTNADRRKAATMRERRRLSKVNEAFETLKRCTSTNPNQRLPKVEILRNAIRYIGSLQALLREQEDAYYPVLEHYSGESDASSPRSNCSDGMMEYSGPPCSSRRRNSYDSSYYTESPNDPKHGKSSVVSSLDCLSSIVERISTDNSTCPILPPAEAVAEGSPCSPQEGASLNDSGAQIPSPTNCTPLPQDSSSSNNPIYQVL

SEQ ID NO: 4 - protein, 319 aa, Sus scrofa
MELLSPPLRDVDLTGPDGSLCNFATADDFYDDPCFDSPDLRFFEDLDPRLVHVGALLKPEEHSHFPAAAHPAPGAREDEHVRAPSGHHQAGRCLLWACKACKRKTTNADRRKAATMRERRRLSKVNEAFETLKRCTSSNPNQRLPKVEILRNAIRYIEGLQALLRDQDAAPPGAAAAFYAPGPLPPGRGGEHYSGDSDASSPRSNCSDGMMDYSGPPSGARRRNCYDGTYYSEAPSEPRPGKNAAVSSLDCLSSIVESISTESPAAPALLLADTPRESSPGPQEAAAGSEVERGTPTPSPDAAPQCPASANPNPIYQVL

SEQ ID NO: 5 - protein, 318 aa, Bos taurus
MELLSPPLRDVDLTGPDGSLCNFATADDFYDDPCFDSPDLRFFEDLDPRLVHVGALLKPEEHSHFPAAAHPAPGAREDEHVRAPSGHHQAGRCLLWACKACKRKTTNADRRKAATMRERRRLSKVNEAFETLKRCTSSNPNQRLPKVEILRNAIRYIEGLQALLRDQDAAPPGAAAAFYAPGPLPPGRSGEHYSGDSDASSPRSNCSDGMMDYSGPPSGARRRNCYDRTYYSEAPNEPRPGKSAAVSSLDCLSSIVERISTESPAAPALLLADAPPESSPGPQEAAGSEVERGTPAPSPDTAPQGLAGANPNPIYQVL

SEQ ID NO: 6 - protein, 300 aa, Oreochromis niloticus
MELSDISFPIPTADDFYDDPCFNTSDMHFFEDLDPRLVHVGLLKPDDSSSSSSSSPSSSSSSPSSLLHLHHHAEVEDDEHVRAPSGHHQAGRCLLWACKACKRKTTNADRRKAATLRERRRLSKVNDAFETLKRCTTANPNQRLPKVEILRNAISYIESLQALLRGGQEDGFYPVLEHYSGDSDASSPRSNCSDGMTDFNGPTCQTTRRGSYDSSSYFSETPNGGLKSERSSVVSSLDCLSSIVERISTDNSSLLPPADGPGSPTTTTTVPVGEAGTAPATAQVSSPTASQDPNLIYQVL

SEQ ID NO: 7 - protein, 524 aa, Gallus gallus
MAALPGTVPRMMRPAPGQNYPRTGFPLEVSTPLGQGRVNQLGGVFINGRPLPNHIRHKIVEMAHHGIRPCVISRQLRVSHGCVSKILCRYQETGSIRPGAIGGSKPRQVATPDVEKKIEEYKRENPGMFSWEIRDRLLKDGHCDRSTVPSVSSISRVLRIKFGKKEEEEDCDKKEEDGEKKAKHSIDGILGDKGNRLDEGSDVESEPDLPLKRKQRRSRTTFTAEQLEELEKAFERTHYPDIYTREELAQRTKLTEARVQVWFSNRRARWRKQAGANQLAAFNHLLPGGFPPTGMPTLPPYQLPDSTYPTTTISQDGGSTVHRPQPLPPSTMHQGGLAAAAAADSSSAYGARHSFSSYSDSFMNAAAPANHMNPVSNGLSPQKQGAQNKMQCSRWNLTIALNNQVMSILSNPSGVPPQPQADFSISPLHGGLDTTNSISASCSQRSDSIKSVDSLPTSQSYCPPTYSTTSYSVDPVAGYQYGQYGQTAVDYLTKNVSLSTQRRMKLGEHSAVLGLLPVETGQAY

SEQ ID NO: 8 - protein, 503 aa, Anas platyrhynchos
MAALPGTVPRMMRPAPGQNYPRTGFPLEVSTPLGQGRVNQLGGVFINGRPLPNHIRHKIVEMAHHGIRPCVISRQLRVSHGCVSKILCRYQETGSIRPGAIGGSKPRVATPDVEKKIEEYKRENPGMFSWEIRDRLLKDGHCDRSTVPSGLVSSISRVLRIKFGKKEEEEDCDKKEEDGEKKAKHSIDGILGDKGNRLDEGSDVESEPDLPLKRKQRRSRTTFTAEQLEELEKAFERTHYPDIYTREELAQRTKLTEARVQVWFSNRRARWRKQAGANQLAAFNHLLPGGFPPTGMPTLPPYQLPDSTYPTTTISQDGGSTVHRPQPLPPSTMHQGGLAAAAAADTGSAYGARHSFSSYSDSFMNAAAPANHMNPVSNGLSPQVMSILSNPSGVPPQPQADFSISPLHGGLDTTNSISASCSQRSDSIKSVDSLPTSQSYCPPTYSTTSYSVDPVAGYQYGQYGQTAVDYLAKNVSLSTQRRMKLGGHSAVLGLLPVETGQAY

SEQ ID NO: 9 - protein, 520 aa, Meleagris gallopavo
MAALPGTVPRMMRPAPGQNYPRTGFPLEVSTPLGQGRVNQLGGVFINGRPLPNHIRHKIVEMAHHGIRPCVISRQLRVSHGCVSKILCRYQETGSIRPGAIGGSKPRQVATPDVEKKIEEYKRENPGMFSWEIRDRLLKDGHCDRSTVPSVSSISRVLRIKFGKKEEEEDCDKKEEDGEKKAKHSIDGILGDKGNRLDEGSDVESEPDLPLKRKQRRSRTTFTAEQLEELEKAFERTHYPDIYTREELEKAFERTHYPDIYTREELAQRTKLTEARVQVWFSNRRARWRKQAGANQLAAFNHLLPGGFPPTGMPTLPPYQLPDSTYPTTTISQDGSSTVHRPQPLPPSTMHQGGLAAAAAADSSSAYGARHSFSSYSDSFMNAAAPANHMNPVSNGLSPQVMSILSNPSGVPPQPQADFSISPLHGGLDTTNSISASCSQRSDSIKSVDSLPTSQSYCPPTYSTTSYSVDPVAGYQYGQYGQTAVDYLAKNVSLSTQRRMKLGEHSAVLGLLPVETGQAY

SEQ ID NO: 10 - protein, 503 aa, Sus scrofa
MAALPGTVPRMMRPAPGQNYPRTGFPLEVSTPLGQGRVNQLGGVFINGRPLPNHIRHKIVEMAHHGIRPCVISRQLRVSHGCVSKILCRYQETGSIRPGAIGGSKPRQVATPDVEKKIEEYKRENPGMFSWEIRDRLLKDGHCDRSTVPSVSSISRVLRIKFGKKEEDDEADKKEDDSEKKAKHSIDGILGDKGNRLDEGSDVESEPDLPLKRKQRRSRTTFTAEQLEELEKAFERTHYPDIYTREELAQRTKLTEARVQVWFSNRRARWRKQAGANQLAAFNHLLPGGFPPTGMPTLPPYQLPDSTYPTTTISQDGGSTVHRPQPLPPSTMHQGGLAAAAAAADTSSAYGARHSFSSYSDSFMNPAAPSNHMNPVSNGLSPQVMSILSNPSAVPPQPQADFSISPLHGGLDSATSISASCSQRADSIKPGDSLPTSQSYCPPTYSTTGYSVDPVAGYQYGQYGQTAVDYLAKNVSLSTQRRMKLGEHSAVLGLLPVETGQAY

SEQ ID NO: 11 - protein, 403 aa, Bos taurus
MAALPGTVPRMVRPAPGQNYPRTGFPLEVSTPLGQGRVNQLGGVFINGRPLPNHIRHKIVEMAHHGIRPCVISRQLRVSHGCVSKILCRYQETGSIRPGAIGGSKPRQVATPDVEKKIEEYKRENPGMFSWEIRDRLLKDGHCDRSTVPSVSSISRVLRIKFGKKEEEDEADKKEEDGEKKAKHSIDGILGDKGNRLDEGSDVESEPDLPLKRKQRRSRTTFTAEQLEELEKAFERTHYPDIYTREELAQRTKLTEARVQVWFSNRRARWRKQAGANQLAAFNHLLPGGFPPTGMPTLPPYQLPDSTYPTTTISQDGGSTVHRPQPLPPSTMHQGGLAAAAAADTSSAYGARHGFSSYSDSFMNPAAASNHMNPVSNGLSPQWSLTVLFGVQGHHLHLIGAED

SEQ ID NO: 12 - protein, 459 aa, Oreochromis niloticus
MATLPGTVPRMVRPAPGQNYPRTGFPLEVSTPLGQGRVNQLGGVFINGRPLPNHIRHKIVEMAHHGIRPCVISRQLRVSHGCVSKILCRYQETGSIRPGAIGGSKPRQVATPDVEKRIEEYKRENPGMFSWEIRDKLLKDGVCDRSTVPSGNRTDDASDVDSEPDLPLKRKQRRSRTTFTAEQLEELEKAFERTHYPDIYTREELAQRTKLTEARVQVWFSNRRARWRKQAGANQLAAFNHLLPGGFPPTGMPTLPTYQLPESSYPSTTLSQEGSSTLHRPQPLPPSSMHQGGLSADSSSAYGLSSNRHSFSSYSDTFMSPSASSNHMNPVGNGLSPQVMSILSNPSAVPSQPQHDFSISPLHSSLESSNPISASCSQRSDTIKSVDSLASSQSYCPPTYSATSYSVDPVTAGYQYSQYGQTAVDYLAKNVSLSTQRRMKLGDHSAVLGLLQVETGQAY

SEQ ID NO: 13 - protein, 397 aa, Gallus gallus
MGRKKIQISRILDQRNRQVTFTKRKFGLMKKAYELSVLCDCEIALIIFNSTNRLFQYASTDMDKVLLKYTEYSEPHESRTNSDILETLKRKGLGLESHELELEEGLDPGEKLRQMNEGMDLTVARPRFYSPVPLPEVPYGSSPSSSSDGALGSASSSPQSQGRPPAFKPTAPKPSGRSPGPMPPGIGYPLFPAGSLNRALATKTPPPLYLGADGQRRSGGSTARPLYSGLQTLNPVLASGSASIPSHSLTGFPFLAPAQAAEFGAGEAPPPPGFLQPGPPAPWQPPRDMAALGASSRIVPTEDLAPGSSPQPHAISIKSERVSPGLGCPSGAPQPSQGLTSLSEAPQGAADLQPRDDYSKGYPYPPPPPRPLAEEQRATAAVPVPARRAQAEDAWQR

SEQ ID NO: 14 - protein, 406 aa, Anas platyrhynchos
MGRKKIQISRILDQRNRQVTFTKRKFGLMKKAYELSVLCDCEIALIIFNSTNRLFQYASTDMDKVLLKYTEYSEPHESRTNSDILETLKRKGLGLDSHELELDEGLDGGEKMRKLNEGMDLTVARPRFYSPVPLPEASYSSSPPAGGDGALGSTSSSPQSQGRPPAFKPSAPKLSGRSPGPMPPGIGYPLFPPGSLNRALATKTPPPLYLGADGRRGEAHGSLASGRSGGSAARPLYPGLQTLSPVLPPGSAGIPNHSLSGFPFLAPAQAEYGAGEAPPPPGFLQPGPPASWQPPRDMAALGTSTRIVPAEDAAPGSSPQHHAISIKSERVSPGLGCPSGTPQPPPGGLTSLNEAPRGSGDLQPRDDYAKGYPYPLGPPRPLAEEQRATTVPVPPRRAQAVDGWQR

SEQ ID NO: 15 - protein, 397 aa, Meleagris gallopavo
MGRKKIQISRILDQRNRQVTFTKRKFGLMKKAYELSVLCDCEIALIIFNSTNRLFQYASTDMDKVLLKYTEYSEPHESRTNSDILETLKRKGLGLESHELELEEGLDPGEKLRQLNEGMDLTVARPRFYSPVPLPEVPYGSSPPSSSDGALGSASSSPQSQGRPPTFKPTAPKPSGRSPGPMPPGIGYPLFPAGSLNRALATKTPPPLYLGADGQRRSGSGAARPLYSGLQTLSPVLASGSAGIPSHSLTGFPFLTPAQAEFGAGEAPPPPGFLQPGPPAPWQPPRDMAALGASSRIIPTEDLAPGSSPQPHTISIKSERVSPGLGCPSGTPQPSPGSLTSLSEAPRGAADLQPRDDYTKGYPYPLPPPRPLAEEQRATATIPVPARRVQAEDAWQR

SEQ ID NO: 16 - protein, 425 aa, Sus scrofa
MPRPISSRGAGPPALKAPVAAGSSEFCAATAAARCVRERQSECPGGADQGYSTQPGTMGRKKIQISRILDQRNRQVTFTKRKFGLMKKAYELSVLCDCEIALIIFNSANRLFQYASTDMDRVLLKYTEYSEPHESRTNTDILETLKRRGVGLDGPELEPDEGLEGPGEKLRRLAGDGGDPALPRPRLYPAAPTMPSPDMVYGALPPPGCDPSGLGEALPAQSRPSPFRPAAPKAGPPGLAHPLFSPSHLASKTPPPLYLATDGRRPDLPGGLAGARGGLSTSRGLYGSLQSPCSTATPGTPLGSFPFLPAGPPEYGLGDPPPPPGLLQPPTLAPWQPSRGDGPTATPTQSSGGRSLGEEGPPARGASSPTPPVSIKSERLSPAPGGPGDFPKTFPYPLLLARPLAEPLRPGPPMRRLPTADGWPR

SEQ ID NO: 17 - protein, 368 aa, Bos taurus
MGRKKIQISRILDQRNRQVTFTKRKFGLMKKAYELSVLCDCEIALIIFNSSNRLFQYASTDMDRVLLKYTEYSEPHESRTITDILETLKRRGVGLDGPELEPDEGLEGPGEKLRRLAGDGGDPALPRPRLYPAAPTMPSPDMVYGALPPPACEPTGLGEALPAQSRPSPFRPAAPKAGPPGLAHPLFSPSHLASKTPPPLYLAADGRRPDLPGGLAGTRGGLSSSRGLYGGLQSPCSTAAPGPPLGSFPFLPAGPPEYGLGDPPPPPGLLQPPTLAPWQPSRADGPPATPTQPSGGRSLGEDGPPARGASSPTPPVSIKSERLSPAPGGPGDFPKTFPYPLLLARPLAEPLRPGPPLRRLPTADGWPR

SEQ ID NO: 18 - protein, 421 aa, Oreochromis niloticus
MGRKKIQISRILDQRNRQVTFTKRKFGLMKKAYELSVLCDCEIALIIFNSTNRLFQYASTDMDKVLLKYTEYSEPHESRTNTDILETLRRKGLGLDGSELDSEESMQVATDKYPLSEGMDLSVARQRFYGPSLLSPEAQFLVSAGCENGFPNSSGSGMASHRPPSFKSLNSRSSSASPAAPHAHTAFMSPHSGIGYSVFSHGNLNRALDMKSPPPLNVGTENLRADGANQAMGATRANHNSARGLLYQGLHSSSSMVAMGKAGLLSHSLGGYGLPSPGASEYSQPGFYHSVSLQRGTVNPWQAAQPPQEPHGPHINPVESSGGCSFPSQSCTPTSPHLPSLNLSIKSERSSPEHMASPTSPPLHHLRQHSPMSNPDSARHTPPDTHPANGTKEFAKTSYPQDQEEGGQSLRQLEMSDGWQR

SEQ ID NO: 19 - protein, 475 aa, Gallus gallus
MVDTEMPFWPVNFGISPVDLSAMDDHMHSFDIKPFTTVDFSSISSPHYEDIPLGRADQTSIDYKYDIKLQDCQSAIKMEPPSPPYFSEKVQLYNKPHEESSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLRMGEDQIKCKHASPLQEQNKEVAIRIFQRCQFRSVEAVQEITEFAKNIPGFVNLDLNDQVTLLKYGVHEIIYTLLASLMNKDGVLISDGQGFMTREFLKSLRKPFCDFMEPKFEFAVKFNALELDDSDLAIFIAVIILSGDRPGLLNVKPIEDIQDNLLQALELQLKLNHPESSQLFAKLLQKMTDLRQIVTEHVQLLQIIKKTETDMSLHPLLQEIYKDLY

SEQ ID NO: 20 - protein, 475 aa, Anas platyrhynchos
MVDTEMPFWPINFGISPVDLSAMDDHTHSFDIKPFTTVDFSSISSPHYEDIPLARTDQTSIDYKYDIKLQDCQSAIKMEPPSPPYFSEKVQLYNKPHEESSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDQIKCKHVSPLQEQNKEVAIRIFQRCQFRSVEAVQEITEFAKNIPGFVNLDLNDQVTLLKYGVHEIIYTLLASLMNKDGVLISDGQGFMTREFLKSLRKPFCDFMEPKFEFAVKFNALELDDSDLAIFIAVIILSGDRPGLLNVKPIEDIQDNLLQALELQLKLNHPESSQLFAKLLQKMTDLRQIVTEHVQLLQIIKKTETDMSLHPLLQEIYKDLY

SEQ ID NO: 21 - protein, 516 aa, Meleagris gallopavo
MSGHYHLGTSAQLIGEDGHCCTYLSPCQNKGSSQSNRAEITMVDTEMPFWPVNFGISPVDLSAMDDHMHSFDIKPFTTVDFSSISSPHYEDIPLGRADQTSIDYKYDIKLQDCQSAIKMEPPSPPYFSEKVQLYNKPHEESSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLRMGEDQIKCKHASPLQEQNKEVAIRIFQRCQFRSVEAVQEITEFAKNIPGFVNLDLNDQVTLLKYGVHEIIYTLLASLMNKDGVLISDGQGFMTREFLKSLRKPFCDFMEPKFEFAVKFNALELDDSDLAIFIAVIILSGDRPGLLNVKPIEDIQDNLLQALELQLKLNHPESSQLFAKLLQKMTDLRQIVTEHVQLLQIIKKTETDMSLHPLLQEIYKDLY

SEQ ID NO: 22 - protein, 504 aa, Sus scrofa
MGETLGDSLIDPESDAFDTLSANISQEVTMVDTEMPFWPTNFGISSVDLSVMDDHSHSFDIKPFTTVDFSSISTPHYEDIPFPRADPMVADYKYDLKLQDYQSAIKVEPVSPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKNIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 23 - protein, 505 aa, Bos taurus
MGETLGDALIDPESEPFAVTVSARTSQEITMVDTEMPFWPTNFGISSVDLSMMDDHSHAFDIKPFTTVDFSSISTPHYEDIPFPRADPMVADYKYDLKLQEYQSAIKVEPVSPPYYSEKTQLYSKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHISPLQEPSKEVAIRIFQGCQFRSVEAVQEITEYAKNIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 24 - protein, 555 aa, Oreochromis niloticus
MQTPGRDFTHQTDHSFSDMVDTQQLLAWPVGFSLNTADLPELEDSSHSLEMKHLSTFDYASISSSSIPSSLSPPLVSSIPSAGVAYDLSPPQSEEHLTNMDYTNMHSYRTQPDMFNSIKLEPESPPQYSDSPVLSKLPDDTSAAALNIECRVCGDKASGFHYGVHACEGCKGFFRRTIRLKLVYDHCDLHCRIHKKSRNKCQYCRFQKCLNVGMSHNAIRFGRMPQAEKEKLLAEFSSDMEHMHPEAADLRALARHLYEAYLKYFPLTKAKARAILSGKTGDNVPFVIHDMKSLMEGEQFINCKQIPVPEHHQQQQHQTSTLPPVHGGYTETHPMSESEVMRMTGFGGHGEADAVELRFFQSCQSRSAEAVREVTEFAKSIPGFVDLDLNDQVTLLKYGVIEVLIIMMSPLMNKDGTLISYGQIFMTREFLKSLRKPFCQMMEPKFEFSVKFNTLELDDSDMALFLAVIILSGDRPGLLNVKPIEQLQETVLHSLELQLKLNHPDSLQLFAKLLQKMTDLRQIVTDHVHLIQLLKKTEIDMCLHPLLQEIMKDLY

SEQ ID NO: 25 - DNA, 5823 bp, synthetic construct
  source 1..5823: mol_type = other DNA
atgagttcggaccaggaaatggccatctttggggaggctgctccttacctccgaaagtctgaaaaggagcgcattgaagcccagaataagccttttgatgccaagacatctgtctttgtggccgagcccaaggaatcttttgtcaaagggactatccagagcagagaaggtgggaaagtgacagtgaagaccgaaggaggagcgactctgacagtgaaagaggatcaagtcttccccatgaatcctcccaaattcgacaagatcgaggacatggccatgatgacccacttgcacgagcctgctgtgctgtacaacctcaaagagcgttatgcagcctggatgatctacacctactcgggcctcttctgcgtcaccgtcaacccctacaagtggctgccggtgtacaaccctgaggtggtgacggcctaccgaggcaaaaagcgccaggaggccccgccccacatcttctccatctctgacaacgcctatcagttcatgctgactgaccgagagaatcagtcaatcctgatcaccggagaatccggggcaggaaagactgtgaacacaaaacgtgtcatccagtactttgcaacaattgcagtcactggggagaagaagaaggaggaaataacttcaggcaaaatacaggggactctggaggatcagatcatcagtgccaatccactgcttgaggcctttggcaacgccaagaccgtgaggaatgacaactcctctcgctttggtaaattcattagaatccactttggcactacaggaaaactggcctctgctgatattgaaacatatctgttagagaagtctagagttactttccagcttaaggctgaaaggagctatcatattttttaccagatcacatcaaacaggaaaccagaactaattgagatgcttctgattaccaccaacccatatgactacccattcatcagtcaaggggagatcagtgtggccagtattgatgatcaggaagaactgatagccacagatagtgctattgatattttgggctttactaatgaagagaaggtctccatctacaagctcacgggggctgtgatgcattatgggaacctgaaattcaagcaaaagcagcgtgaggagcaagcagagccagatggcactgaagtggctgacaaggctgcctacctccagagtctgaactctgctgacctactcaaagccctttgctaccccagggtcaaggttggcaatgagtatgtcaccaaaggccagactgtagagcaggtaaccaatgcagtgggtgctctggccaaagccgtctatgagaagatgttcctgtggatggtcgcccgcatcaaccagcagctggacaccaagcagcccaggcagtacttcatcggggtcttggacatcgctggctttgagatctttgatttcaacagcctggagcagctgtgcatcaacttcaccaatgagaaactgcaacagtttttcaaccaccacatgttcgtgctggagcaggaggagtacaaaagagaaggcatcgagtggacgttcattgactttgggatggacctggctgcctgcatcgagctcattgagaagcctatgggcatcttctccatcctcgaagaggagtgcatgttccccaaggccacagacatgtccttcaagaacaagctgtatgaccagcacctgggcaagtctgccaatttccagaagcccaaggtggtcaagggcaaacctgaggcccattttgccctgatccactatgcgggtgtcgtggactacaacattactggctggctggagaagaacaaggaccccctgaacgatactgtggttgggctgtaccagaagtctgcgttgaaaactctagctttccttttctctgggactccaactggtgactcagaggcaagtggtggaactaagaaaggtggtaagaagaagggctcttctttccagacggtgtctgcacttttcagggaaaatctgaacaaactgatgaccaacctcaggagtacccatcctcactttgtgcggtgcatcatccccaatgaaacgaagacccctggggccatggagcacgagctggtcctgcaccagctgaggtgtaatggcgtgctagagggcatccgcatctgcaggaagggcttccccagcagaatcctgtatgcggacttcaaacagagatacaaggtattaaatgcaagtgcgatccctgaaggacaatacatcgacagcaagaaggcttctgagaagctccttgcatccattgacatcgaccacacccagtataaatttggtcacaccaaggtctttttcaaagctggtcttctggggctcctagaggagatgcgagatgaaaagctagcccagctgatgacccgaacccaggccaggtgcagagggttcttggcaagagtggagtaccagaagatggtggagaggagggagtccatcttctgcatccagtacaacatccgagccttcatgaatgtcaagcactggccctggatgaaactgttcttcaggatcaagcccctgctgaagagtgcagagacggagaaggagatggccaccatgaaggaagagttccagaaaaccaaagatgaactggccaagtcagaggccaaaaggaaggaactggaggaaaaaatggtgactctcttgaaagaaaaaaatgacctacagctccaggttcaatctgaagctgaaggcttggctgatgcagaggaaagatgtgaccagctgatcaaaaccaaaatccagctggaggctaagatcaaggaggtgactgagagagcagaggatgaggaagagatcaatgctgagctgacggccaagaagaggaaactggaggacgaatgttcggaactaaagaaagacatagacgaccttgagctgacactggccaaggttgagaaggagaaacatgccacagaaaataaggtgaaaaacctcacagaagagatggcaggtctggatgaaaccatcgccaagctgaccaaggagaaaaaggccctccaggaggcccaccagcagaccctggatgacctgcaggcagaagaggacaaagtcaacactctgaccaaagctaaaaccaagctagagcagcaagtggatgaccttgaggggtccttggagcaagaaaagaaacttcgcatggacctagagagagccaagaggaaacttgaaggtgatctcaagttggcccaagagtccataatggacattgaaaatgaaaaacaacaacttgatgaaaagctcaaaaagaaagagtttgaaatcagcaatttgcaaagcaagattgaagatgaacaagcacttggcattcaactgcagaagaagatcaaagagttgcaggcccgcatcgaggagctggaggaggagatcgaggccgagcgggcctcccgcgccaaggcagagaagcagcgctcagacctctcccgggaactggaggagatcagcgagcggctagaagaagccggcggggccacttccgcccagattgagatgaacaagaagcgggaggccgagttccagaagatgcgcagggacctggaggaggccaccctgcagcacgaggccacggcggccgctcttcgcaagaagcacgcggacagtgtggctgagctgggggagcagattgacaacctgcagagggtcaagcagaagctggagaaggagaagagcgagatgaagatggagatcgacgaccttgctagcaatgtggaaacgatctctaaagccaagggaaacctagagaagatgtgccgaactctggaggaccaagtgaacgagctgaaatccaaggaggaggagcagcagcggctgatcaatgacctgacaacccagaggggacgcttgcagactgaatctggtgaattttcacgtcagctagatgagaaagaagctctggtgtctcagttgtcaaggggcaaacaagcatttactcaacagattgaggaattaaagaggcaacttgaagaggagataaaggccaagaatgccctggcccatggcctgcagtcagcccgtcatgactgtgacctgctgcgtgaacagtatgaggaggagcaggaatccaaggccgagctgcagagggcgctgtccaaggccaacaccgaggtggcccagtggaggaccaagtatgagacagacgccatccagcgcacggaggagctggaggaggccaagaagaaactggcccagcgtctgcaggctgctgaggaacacgtggaagctgtgaacgccaaatgcgcctcccttgagaagaccaagcagaggctccagaatgaggttgaggacctcatgctcgatgtggagaggacaaacgctgcctgcgcagccctggacaaaaagcaaaggaactttgacaagatcctggcagaatggaaacagaagtatgaggaaacccacgccgagctcgaggcagctcagaaggaggcccgctctctgggtaccgagctgttcaagatgaagaatgcctacgaggagtccttggatcagctagaaactttgaaacgagagaacaaaaacttgcagcaggagatctctgacctcacggagcagatcgcagaaggagggaaacgtatgcatgaactggagaaaataaagaagcaagtggaacaagaaaagtctgaaattcaggctgctttagaggaggcagaggcatctcttgaacacgaagagggcaagatcctgcgtatccagctggagctgaaccaagtcaagtctgaaattgacaggaaaattgctgaaaaggatgaggaaattgaccagctgaagaggaaccacattagagtcgtggagtccatgcagaccatgctggatgctgaaatcaggagcaggaatgacgccatcaggctcaagaagaagatggaaggagacctcaatgaaatggagatccagttgaaccatgccaaccgcatggctgctgaggccctgaagaactacaggaacactcaagccatcctcaaggatacccagatccacctggatgatgctctgcggggccaggaggacttgaaggagcagctggccatggtggagcgcagagccaacctgctgcaggctgagatcgaggagctgagggccaccctggagcagacagagaggagcaggaagatcgcagagcaggagctcctggatgccagtgagcgcgtccagctcctgcacacccagaacaccagcctgatcaacaccaagaagaagctagagacagacatcacgcaaatccagggagagatggaggacattctccaggaagctcgcaacgcagaagagaaagccaagaaggccatcactgatgcagccatgatggctgaggagctgaagaaggagcaggacaccagcgcccacctggagcggatgaagaagaacatggagcagacggtgaaggacctgcagaaccgtctggatgaggctgagcagctggccctgaagggcgggaagaagcaaatccagaaactggaggccagggtgcgtgagctggaaggagaggtagagagtgagcagaagcgcaatgtggaagctgtcaagggtctgcgcaaacacgagaggagagtgaaggaactcacttaccagacggaagaagatcggaaaaatattctcagacttcaggacttggtagataaacttcaggcgaaagtgaaatcttacaagagacaagctgaggaggctgaggaacaatccaatacaaatctatctaaattccgcaagctccagcatgagctggaggaagccgaggaaagggctgacattgctgaatcccaggtcaacaagctgcgggtgaagagccgggaggttcacacaaaaatcatcagtgaagagtga

SEQ ID NO: 26 - protein, 1940 aa, synthetic construct
MSSDQEMAIFGEAAPYLRKSEKERIEAQNKPFDAKTSVFVAEPKESFVKGTIQSREGGKVTVKTEGGATLTVKEDQVFPMNPPKFDKIEDMAMMTHLHEPAVLYNLKERYAAWMIYTYSGLFCVTVNPYKWLPVYNPEVVTAYRGKKRQEAPPHIFSISDNAYQFMLTDRENQSILITGESGAGKTVNTKRVIQYFATIAVTGEKKKEEITSGKIQGTLEDQIISANPLLEAFGNAKTVRNDNSSRFGKFIRIHFGTTGKLASADIETYLLEKSRVTFQLKAERSYHIFYQITSNRKPELIEMLLITTNPYDYPFISQGEISVASIDDQEELIATDSAIDILGFTNEEKVSIYKLTGAVMHYGNLKFKQKQREEQAEPDGTEVADKAAYLQSLNSADLLKALCYPRVKVGNEYVTKGQTVEQVTNAVGALAKAVYEKMFLWMVARINQQLDTKQPRQYFIGVLDIAGFEIFDFNSLEQLCINFTNEKLQQFFNHHMFVLEQEEYKREGIEWTFIDFGMDLAACIELIEKPMGIFSILEEECMFPKATDMSFKNKLYDQHLGKSANFQKPKVVKGKPEAHFALIHYAGVVDYNITGWLEKNKDPLNDTVVGLYQKSALKTLAFLFSGTPTGDSEASGGTKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQYIDSKKASEKLLASIDIDHTQYKFGHTKVFFKAGLLGLLEEMRDEKLAQLMTRTQARCRGFLARVEYQKMVERRESIFCIQYNIRAFMNVKHWPWMKLFFRIKPLLKSAETEKEMATMKEEFQKTKDELAKSEAKRKELEEKMVTLLKEKNDLQLQVQSEAEGLADAEERCDQLIKTKIQLEAKIKEVTERAEDEEEINAELTAKKRKLEDECSELKKDIDDLELTLAKVEKEKHATENKVKNLTEEMAGLDETIAKLTKEKKALQEAHQQTLDDLQAEEDKVNTLTKAKTKLEQQVDDLEGSLEQEKKLRMDLERAKRKLEGDLKLAQESIMDIENEKQQLDEKLKKKEFEISNLQSKIEDEQALGIQLQKKIKELQARIEELEEEIEAERASRAKAEKQRSDLSRELEEISERLEEAGGATSAQIEMNKKREAEFQKMRRDLEEATLQHEATAAALRKKHADSVAELGEQIDNLQRVKQKLEKEKSEMKMEIDDLASNVETISKAKGNLEKMCRTLEDQVNELKSKEEEQQRLINDLTTQRGRLQTESGEFSRQLDEKEALVSQLSRGKQAFTQQIEELKRQLEEEIKAKNALAHGLQSARHDCDLLREQYEEEQESKAELQRALSKANTEVAQWRTKYETDAIQRTEELEEAKKKLAQRLQAAEEHVEAVNAKCASLEKTKQRLQNEVEDLMLDVERTNAACAALDKKQRNFDKILAEWKQKYEETHAELEAAQKEARSLGTELFKMKNAYEESLDQLETLKRENKNLQQEISDLTEQIAEGGKRMHELEKIKKQVEQEKSEIQAALEEAEASLEHEEGKILRIQLELNQVKSEIDRKIAEKDEEIDQLKRNHIRVVESMQTMLDAEIRSRNDAIRLKKKMEGDLNEMEIQLNHANRMAAEALKNYRNTQAILKDTQIHLDDALRGQEDLKEQLAMVERRANLLQAEIEELRATLEQTERSRKIAEQELLDASERVQLLHTQNTSLINTKKKLETDITQIQGEMEDILQEARNAEEKAKKAITDAAMMAEELKKEQDTSAHLERMKKNMEQTVKDLQNRLDEAEQLALKGGKKQIQKLEARVRELEGEVESEQKRNVEAVKGLRKHERRVKELTYQTEEDRKNILRLQDLVDKLQAKVKSYKRQAEEAEEQSNTNLSKFRKLQHELEEAEERADIAESQVNKLRVKSREVHTKIISEE

SEQ ID NO: 27 - DNA, 5823 bp, synthetic construct
  source 1..5823: mol_type = other DNA
atgagcagcgaccaagaaatggcgatatttggcgaggccgccccctacctgagaaagagcgagaaggagagaatcgaggctcagaacaagcccttcgacgccaagacaagcgtgttcgtggccgagcccaaggagagcttcgtgaagggcaccattcagagcagagaaggcggtaaagtgacggtgaagaccgagggcggcgccaccttaactgtaaaggaggaccaagtgttccccatgaacccccccaagttcgataaaatcgaggacatggccatgatgacccacctgcacgagcccgccgtgctgtacaacctgaaggagagatacgccgcctggatgatctacacctacagcggcctgttctgcgtgaccgtgaacccctacaagtggctgcccgtgtacaaccccgaggtggtgaccgcctacagaggcaagaagagacaagaggcccccccccacatcttttctatcagcgataacgcctatcagttcatgctgaccgacagagagaatcaatctatcctcatcaccggcgagagcggcgccggcaagaccgtgaacaccaagagagtgattcagtacttcgccaccatcgccgtgaccggcgagaagaagaaagaagagataacgagcggcaagatccaaggcactctcgaggaccaaattattagcgctaaccccctgctggaggccttcggcaacgccaagaccgtgagaaacgacaatagctcgcggtttggcaagttcatcagaatccacttcggcaccaccggcaagctggctagcgccgacatcgagacctacctgctggagaagagcagagtgacctttcagctgaaggccgagagaagctaccacatcttctatcagatcacaagcaacagaaagcccgaactcatagagatgttactgatcaccaccaacccctacgactaccccttcatcagccaaggggagatcagcgtggctagcattgacgaccaagaggagctgatcgccaccgacagcgccatcgacatcctgggcttcaccaacgaagagaaggtgagcatctacaagctgaccggcgccgtgatgcactacggcaacctgaaattcaaacagaaacagagagaggagcaagccgagcccgacggcactgaagtggccgataaggccgcctacctgcagagcctgaacagcgccgacctgctgaaggccctgtgctacccaagagtgaaggtgggcaacgagtacgtgaccaagggacagaccgtggagcaagtgaccaacgccgtgggcgcactggccaaagccgtttacgagaagatgttcctgtggatggtggctagaatcaaccaacaactggacactaagcagccaagacagtacttcatcggagtgcttgacatcgccggcttcgagatcttcgacttcaactcgttagaacaactgtgcatcaactttacgaacgaaaagttacagcagttcttcaaccaccacatgttcgtgcttgagcaagaggaatataagagagagggcatcgagtggaccttcatcgacttcggcatggacctggccgcctgcatcgagcttatagaaaagccaatgggcatcttcagcattctggaagaagagtgcatgttccccaaggccaccgacatgagcttcaagaacaagctgtacgatcagcacctgggcaagagcgccaactttcagaagcccaaggtggtgaagggcaagcccgaggcccacttcgccctgatccactacgccggcgtggtggactacaacatcaccggctggctggagaaaaacaaggacccactgaacgacaccgtggtgggcctgtatcagaagagcgccctgaagactctggccttcctgttctccggcacccccaccggcgactcggaggctagcggcggcaccaagaagggcggtaaaaaaaaaggcagcagctttcagaccgtgagcgccctgttcagagagaacctgaataaactgatgaccaacttgagaagcacccacccccacttcgtgagatgcatcatccccaacgagacgaaaacaccgggcgccatggagcacgagttagtgctgcatcagctgagatgcaacggcgtactggagggcatcagaatctgcagaaagggcttcccaagcagaatcctgtacgccgacttcaagcagagatacaaggtgctgaacgctagcgccatccccgagggacagtacatcgacagcaagaaggctagcgagaagctgttagctagcatcgacatcgaccacacacagtacaagttcggccacaccaaggtgttcttcaaggccggcctgctgggcctgctagaagagatgcgtgacgaaaaactcgcccaattaatgacaagaacgcaagctagatgcagaggcttcctggctagagtggagtatcagaagatggtggagagaagagagagtatcttctgcattcagtacaacatcagagccttcatgaacgtgaagcactggccctggatgaagctgttcttcagaatcaagcccctgctgaagagcgccgagaccgagaaagagatggccacgatgaaggaggagtttcagaagacaaaagacgagctggccaagagcgaggctaagagaaaggagctagaggagaaaatggtgaccctgctgaaggagaagaacgacctgcagctgcaagtgcagagcgaggccgagggcctggccgatgccgaggaacgatgtgatcagctgatcaagaccaagattcagttggaggccaagattaaagaagtgaccgagagagccgaggatgaagaggagatcaatgccgagctgaccgccaagaaacggaagctcgaagacgagtgcagcgaactgaagaaggacatagatgacttagagctgacgttggccaaggtcgagaaggagaagcatgccaccgagaacaaggtgaagaacctgaccgaggagatggccggcctggacgagaccatcgccaagctgaccaaggagaagaaggcccttcaagaggcccatcagcagaccctggacgacctgcaagccgaagaggataaagtaaacaccctgaccaaggccaagaccaagctggagcagcaagtggacgacctggagggcagtctggaacaagagaagaagctcagaatggacctggagagagccaagcgaaaattggagggtgatttgaaactggcccaagagagcatcatggacatcgagaacgagaagcagcaacttgatgaaaaattaaagaaaaaggagttcgagatcagcaacctgcaaagtaagattgaagacgaacaagcgctgggcattcagctgcagaagaagattaaggagttacaagctagaattgaagaactcgaggaagaaatcgaagccgagagggctagccgcgccaaggccgagaaacaaagatcggatctgagcagagaactagaagagatctctgaaagactggaagaggccgggggggccacgtcagctcagatcgagatgaacaagaagagagaggccgagttccaaaaaatgagaagagacctcgaggaggcgacgcttcaacatgaagctaccgccgccgccctgagaaagaagcacgccgacagcgtggccgagctgggcgagcagatcgacaacctgcagagagtgaagcagaagttggagaaagaaaagtcggaaatgaagatggagatcgacgatctggctagcaacgtggagaccatcagcaaggccaagggcaacctggagaagatgtgcagaaccctggaagaccaagtgaacgaactgaagagcaaggaagaggaacagcagaggctgatcaacgacctgaccacacagagaggcagactgcagaccgagagcggcgagttcagcagacagctggacgagaaggaggccctggtgtctcagctgagcagaggcaagcaagccttcacacagcagatcgaggagctgaaaagacagcttgaagaagagataaaggcaaagaacgccctggcccacggcctgcagagcgctagacacgactgcgacctgctgagagagcagtatgaagaggaacaagagtcgaaagccgagctgcagagagccctgagcaaggccaacaccgaggtcgctcaatggagaaccaagtacgagaccgacgccattcagagaaccgaggagctcgaagaagccaaaaaaaagctggctcaacgactgcaagccgctgaggagcacgttgaggcagtgaacgccaagtgcgcatcactggagaaaaccaagcagcgtctgcaaaacgaagtggaggacctgatgctggacgtggagagaaccaacgccgcctgcgccgccctggacaagaagcagagaaacttcgacaagatcctggccgagtggaagcagaagtacgaggagacccacgccgagctggaggccgctcagaaagaggcccgaagcctgggcaccgagctgttcaagatgaagaacgcctacgaggagagcctggatcagctggagaccctgaagagagagaacaagaacctgcagcaagagatctctgacttgaccgagcagatcgccgagggtggtaagaggatgcatgagttggagaaaatcaagaagcaagtcgaacaagagaaaagtgagatccaagccgccctggaagaggcggaagcatcgctcgaacatgaagagggcaagatcctgaggattcagctggagctcaaccaagtgaagagcgagatcgacagaaagattgccgagaaagacgaggagattgatcagctgaagagaaaccacatcagagtggtggagagcatgcagaccatgctggacgccgagatcagaagcagaaacgacgccatcagactgaagaagaagatggagggcgacctgaatgaaatggagattcagctgaaccacgccaacagaatggccgccgaggccctgaagaactacagaaacacccaagccatcctgaaggacacacagattcacctggacgacgccctgagaggccaagaggacctgaaggagcagctggccatggtggaacgaagagctaacctgctgcaagccgagatagaagagctccgagccaccctggagcaaacagaacggagcagaaaaatcgccgagcaagagctgctggacgctagcgagagagtgcaactgctgcatacacagaacacaagcctgatcaacaccaagaagaaactggagaccgacatcacgcagatccaaggcgagatggaagacatcctccaagaggctagaaatgccgaagagaaggccaagaaggccatcaccgacgccgccatgatggccgaagagctgaaaaaagagcaagacacgtccgcgcacctagagagaatgaagaagaacatggagcagaccgtgaaggatctgcagaacagactggacgaggccgagcaactggccctgaaggggggaaagaaacagattcagaaacttgaagcaagggtgcgtgagttagagggcgaggtggagagcgagcagaagagaaacgttgaggcggtgaagggtctgagaaagcacgagagaagagtgaaggagctgacctatcagaccgaagaagaccgcaaaaacatcctgagactgcaagacctggtggacaagctgcaagccaaggtgaagagctacaagagacaagccgaggaggcagaagagcaatcgaacaccaacctgagcaagttcagaaagctgcaacacgagctggaggaagcggaggaaagagccgacatcgccgagagccaagtgaacaagctgagggtgaaaagtagagaggtacataccaagataatcagcgaggagtga

SEQ ID NO: 28 - protein, 1940 aa, synthetic construct; identical to SEQ ID NO: 26

SEQ ID NO: 29 - DNA, 666 bp, synthetic construct
  source 1..666: mol_type = other DNA
atccagtttgatacggttcggatggggagcccctataacttggggctccttcaatagccaaactgcgcaggcggtgtccccccaccccccacccgccccgccctgcgactttgagctgggtccagacctagcatcctaattctctactagcccgtgaggtcagaggcagcacctccattgtaacgcgactagagcagggcggcgtcaacaccaccgcccgaagtcccgacccaccagcccctcctaccgcttcccctcccattacccctttccgagacagtgccagctagcaagtcccagaagagtctggcgagttctgggaggagtggcatccagggcgccgattggtcccagaaagccagggggcaggacttgaggcgaaacccctggaatattcccgacctggcagccccactgaactcggtcattggctgacgagggaaaaggcggggcttgatgaagaattataaacacagagccgcctgaggagaaacagcagcctggagagagctgataaaacttgcggcttagtccgtgagaacagcttccgcagacccgctatctccaaggaccgccccgaggggcaccagagcttcacgtcgttgatcctgtgggccgttttcaggtttgaagcttatctcggagccgaaaaggcagggcaccggc

SEQ ID NO: 30 - DNA, 1062 bp, synthetic construct
  source 1..1062: mol_type = other DNA
atggagtcggccgacttctacgaggcggagccgcggcccccgatgagcagccacctccagagccctccacacgctcctagcagcgcagctttcggctttcctagaggcgctggccctagccagccacctgccccacctgccgcccctgagcctctgggcggcatctgcgaacacgagacgtccatcgacatcagcgcctacatcgacccggccgccttcaacgacgagttcctggccgacctgttccaacacagccggcagcaggagaaggccaaggctgcagctgcacctgcaggaggcggaaacgactttgactaccctggcgcccctgtgggccctggaggcgccgtcatgcctggagggacacacggtccccctcctggctacggctgcgctgcagctggctacctggacagcaggctggagcctctgtacgagcgggtcggggcgccggcgctgcggccgctggtgatcaagcaggagccgcgcgaggaggacgaagctaagcagctggctctggctggactctttccttaccagccacctccacctccacctccacctcacagccaccctccacctgctcacctggccgctcctcacctgcagttccagatcgcacactgcggccagaccaccatgcacctgcagcctggccaccctgctcctccacctacacctgtgcctagccctcacccagctcctgcactcggagccgctggcctgccaggacctggaggcgctctcaaggggctggccgccacacaccccgacctccgtgctggaggcggagggggaggcaaagccaagaagtccgtggacaagaacagcaacgagtacagggtgaggcgcgagcgcaacaacatcgcggtgcgcaagagccgggacaaggccaagcagcgcaacgtggagacgcagctgaaggtgctggagctgaccagtgacaatgaccgcctgcgcaagcgggtggaacaactgagccgcgaactggacacgctgcggggcaccttccgtcagctgcccgagagctccctggtcaaggccatgggcaactgcgcgtga

SEQ ID NO: 31 - protein, 353 aa, synthetic construct
MESADFYEAEPRPPMSSHLQSPPHAPSSAAFGFPRGAGPSQPPAPPAAPEPLGGICEHETSIDISAYIDPAAFNDEFLADLFQHSRQQEKAKAAAAPAGGGNDFDYPGAPVGPGGAVMPGGTHGPPPGYGCAAAGYLDSRLEPLYERVGAPALRPLVIKQEPREEDEAKQLALAGLFPYQPPPPPPPPHSHPPPAHLAAPHLQFQIAHCGQTTMHLQPGHPAPPPTPVPSPHPAPALGAAGLPGPGGALKGLAATHPDLRAGGGGGGKAKKSVDKNSNEYRVRRERNNIAVRKSRDKAKQRNVETQLKVLELTSDNDRLRKRVEQLSRELDTLRGTFRQLPESSLVKAMGNCA